(19) United States
(12) Patent Application Publication
(10) Pub. No.: US 2025/0270173 A1
(43) Pub. Date: Aug. 28, 2025

(54) PYRIDINE DERIVATIVES FOR TREATING PSYCHIATRIC DISORDERS

(71) Applicant: GILGAMESH PHARMACEUTICALS, INC., New York, NY (US)

(72) Inventors: Brian Thomas O'NEILL, Haddam, CT (US); Jotham Wadsworth COE, Niantic, CT (US); Andrew Carry KRUEGEL, Millington, NJ (US)

(73) Assignee: GILGAMESH PHARMACEUTICALS, INC., New York, NY (US)

(21) Appl. No.: 18/857,979

(22) PCT Filed: Apr. 18, 2023
(86) PCT No.: PCT/US2023/018899

§ 371 Date: Oct. 18, 2024

Related U.S. Application Data
(60) Provisional application No. 63/332,324, filed on Apr. 19, 2022.

(51) Int. Cl. C07D 241/18; A61K 31/4418; A61K 31/4965; C07D 213/69; C07D 213/70

(52) U.S. Cl. CPC C07D 241/18; A61K 31/4418; A61K 31/4965; C07D 213/69; C07D 213/70

(57) ABSTRACT

The present disclosure relates to a compound of the formula; or a pharmaceutically acceptable salt thereof, wherein R₁, R₂, R₃, R₄, R₅, and R₆ are as defined herein. It also relates to pharmaceutical compositions comprising a compound of Formula I and to the use of the compound of Formula I for treating psychiatric disorders.

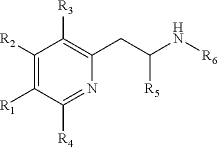

### FIELD OF THE DISCLOSURE

The present disclosure relates to aminoalkyl-pyridine derivatives for the treatment of different medical conditions that are treated by activation of the serotonin receptors, for example, mental illnesses and other disorders and conditions in the field of psychiatry. The present disclosure also relates to various methods for making these aminoalkyl-pyridine derivatives and corresponding intermediates and methods of use of these aminoalkyl-pyridine derivatives for the treatment of psychiatric disorders.

### BACKGROUND OF THE DISCLOSURE

Psychiatric disorders are mental disorders that cause abnormal thinking and perceptions. People with psychoses lose touch with reality. Also known as mental illness and mental disorders, psychiatric disorders include a wide range of disorders that include, but are not limited to, depressive disorders, anxiety and panic disorders, schizophrenia, eating disorders, substance use disorders, post-traumatic stress disorder, attention deficit/hyperactivity disorder, and obsessive-compulsive disorder. The severity of symptoms varies such that some individuals experience debilitating disease that precludes normal social function, while others suffer with milder symptoms or with intermittent repeated episodes across their lifespan. Although the presentation and diagnostic criteria among mental illness conditions are distinct in part, there are common endophenotypes of note across the diseases, and often comorbidities exist. Specifically, there exist endophenotypes associated with alterations in mood, cognition, and behavior. Interestingly, many of these endophenotypes extend to neurological conditions as well. For example, attentional deficits are reported in patients with attention deficit disorder, attention deficit hyperactivity disorder, eating disorders, substance use disorders, schizophrenia, depression, obsessive compulsive disorder, traumatic brain injury, Fragile X, Alzheimer's disease, Parkinson's disease, and frontotemporal dementia.

Depression and anxiety are two of the most common psychiatric disorders worldwide. Depression is a multifaceted condition characterized by episodes of mood disturbances alongside other symptoms such as anhedonia, psychomotor complaints, feelings of guilt, attentional deficits, and suicidal tendencies, all of which can range in severity. According to the World Health Organization, the discovery of mainstream antidepressants has largely revolutionized the management of depression, yet up to 60% of patients remain inadequately treated. This is often due to the drugs' delayed therapeutic effect (generally 6 weeks from treatment onset), side effects leading to non-compliance, or inherent non-responsiveness to them. Similarly, anxiety disorders are a collective of etiologically complex disorders characterized by intense psychosocial distress and other symptoms depending on the subtype. Anxiety associated with life-threatening disease is the only anxiety subtype that has been studied in terms of psychedelic-assisted therapy. This form of anxiety affects up to 40% of individuals diagnosed with life-threatening diseases like cancer. It manifests as apprehension regarding future danger or misfortune accompanied by feelings of dysphoria or somatic symptoms of tension, and often coexists with depression. It is associated with decreased quality of life, reduced treatment adherence, prolonged hospitalization, increased disability, and hopelessness, which overall contribute to decreased survival rates. Pharmacological and psychosocial interventions are commonly used to manage this type of anxiety, but their efficacy is mixed and limited such that they often fail to provide satisfactory emotional relief.

Receptors for serotonin (5-hydroxytryptamine, 5-HT) are an important class of G protein-coupled receptors. Serotonin is thought to play a role in processes related to learning and memory, sleep, thermoregulation, mood, motor activity, pain, sexual and aggressive behaviors, appetite, neurodegenerative regulation, and biological rhythms. Not surprisingly, many mental health disorders, as well as neurological disorders, are impacted by alterations, dysfunction, degeneration, and/or damage to the brain's serotonergic signaling system, which may explain, in part, common endophenotypes and comorbidities among neuropsychiatric and neurological diseases. For example, dysfunction of serotonin signaling has been linked to pathophysiological conditions such as anxiety, depression, obsessive compulsive disorders, schizophrenia, suicide, autism, migraine, emesis, alcoholism, neurodegenerative disorders, chronic pain, existential pain, bipolar disorder, obsessive-compulsive disorder, and smoking. Many therapeutic agents that modulate serotonergic function are commercially available, including serotonin reuptake inhibitors, selective serotonin reuptake inhibitors (SSRIs), and monoamine oxidase inhibitors, and, while primarily developed for depressive disorders, many of these therapeutics are used across multiple other medical indications including, but not limited to, depression in Alzheimer's disease and other neurodegenerative disease, chronic pain, existential pain, bipolar disorder, obsessive-compulsive disorder, anxiety disorders, and smoking cessation. However, in many cases, the marketed drugs show limited benefit compared to placebo, can take six weeks or more to work, and for some patients, are associated with several side effects including trouble sleeping, drowsiness, fatigue, weakness, changes in blood pressure, memory problems, digestive problems, weight gain, and sexual problems. Thus, there is a need in the industry for more effective drugs for the treatment of these disorders.

The field of psychedelic neuroscience has witnessed a recent renaissance following decades of restricted research due to tight legal controls. Psychedelics are one of the oldest classes of psychopharmacological agents known to man and cannot be fully understood without reference to various fields of research, including anthropology, ethnopharmacology, psychiatry, psychology, sociology, and others. Psychedelics (serotonergic hallucinogens), such as lysergic acid diethylamide (LSD), psilocybin, N,N-dimethyltryptamine (DMT), and mescaline, are powerful psychoactive substances that alter sensory perception and mood and affect numerous cognitive processes. They are capable of inducing profound distortions in visual and auditory processing, along with an altered sense of self and relationship of the self to the outside world, described by many users as mystical experiences. They are generally considered physiologically safe and do not lead to dependence or addiction. Their origin predates written history, and they were employed by early cultures in many sociocultural and ritual contexts. After the virtually contemporaneous discovery of LSD and the identification of serotonin in the brain, early research focused intensively on the possibility that LSD and other psychedelics had a serotonergic basis for their action. Today there is a consensus that classical psychedelics are agonists or partial agonists at brain 5-hydroxytryptamine (serotonin) 2A (5-HT2A) receptors. Several useful rodent models have been developed over the years to help understand the neurochemical effects of 5-HT2A receptor activation in the brain, and a variety of imaging techniques have been employed to identify key brain areas that are directly affected by psychedelics.

Psychedelics induce both acute effects and persisting effects long after their acute effects have subsided, which include changes in mood and brain function. Long-lasting effects may result from their unique receptor affinities, which affect neurotransmission via neuromodulatory systems that serve to modulate brain activity, i.e., neuroplasticity, and promote cell survival, are neuroprotective, and modulate brain neuroimmune systems. The mechanisms which lead to these long-term neuromodulatory changes are still under investigation, but likely include epigenetic modifications, gene expression changes, and modulation of pre- and post-synaptic receptor densities. Thus, previously under-researched, psychedelic drugs may potentially provide the next-generation of neurotherapeutics, where treatment-resistant psychiatric and neurological diseases, e.g., depression, post-traumatic stress disorder, dementia, and addiction, may become treatable with attenuated pharmacological risk profiles. Randomized controlled clinical studies have confirmed the antidepressant and other therapeutic effects of classic psychedelics in humans. Importantly, these therapeutic effects are induced rapidly (hours to days) and are often more robust and durable compared to the effects of marketed serotonergic antidepressants (e.g., SSRIs). From such studies, it is evident that agonists of the 5-HT2A receptor are likely to represent a new class of rapid-acting and highly efficacious antidepressants, which will be particularly useful for treating patients that are ineffectively managed by conventional methods.

Generally, the psychedelic treatment model consists of administering a psychedelic drug to induce a mystical experience with a duration depending on the psychedelic, but often lasting four hours or more in the case of psilocybin, currently the most commonly used agent for this purpose. This enables participants to work through and integrate difficult feelings and situations, leading to enduring antidepressant and anxiolytic effects. Classical psychedelics like psilocybin and LSD are being studied as potential candidates. In one study with classical psychedelics for the treatment of depression and anxiety associated with life-threatening disease, it was found that, in a supportive setting, psilocybin consistently produced significant and sustained antidepressant and anxiolytic effects.

Further, emerging clinical research and evidence suggests that psychedelic-assisted therapy also shows potential as an alternative treatment for refractory substance use disorders and other mental health conditions. For example, recent studies suggest that psilocybin-assisted therapy is useful for treating alcohol use disorder. Also recently, it has been found that psilocybin-assisted therapy may be useful for treating tobacco use disorder, demonstrating abstinence rates of 80% at 6-month follow-up and 67% at 12-month follow-up; such rates are considerably higher than any documented in the tobacco cessation literature. These results coincide with bourgeoning evidence from recent clinical trials lending support to the effectiveness of psilocybin-assisted therapy for treatment-resistant depression and end-of-life anxiety. Research on the potential benefits of psychedelic-assisted therapy for opioid use disorder (OUD) is beginning to emerge, and accumulating evidence supports a need to advance this line of investigation. Available evidence from earlier randomized clinical trials suggests a promising role for treating OUD: higher rates of abstinence were observed among participants receiving high-dose LSD for heroin addiction compared to controls at long-term follow-ups.

Psychosis is often referred to as an abnormal state of mind that is characterized by hallucinatory experiences, delusional thinking, and disordered thoughts. Moreover, this state is accompanied by impairments in social cognition, inappropriate emotional expressions, and bizarre behavior. Most often, psychosis develops as part of a psychiatric disorder, and it represents an integral part of schizophrenia. It corresponds to the most florid phase of the illness. The very first manifestation of psychosis in a patient is referred to as first-episode psychosis. It reflects a critical transitional stage toward the chronic establishment of the disease, which is presumably mediated by progressive structural and functional abnormalities seen in diagnosed patients. Anecdotal evidence suggests that low, non-hallucinogenic doses (microdosing) of psychedelics that are administered regularly can reduce symptoms of schizophrenia and psychosis.

Despite this great therapeutic promise, existing psychedelic drugs face several challenges in both their pharmacology and delivery that must be addressed to maximize safety and increase adoption in clinical practice. Although psychedelics are generally considered to be safe from a physiological perspective, some somatic side effects have been reported. For example, psilocybin, when administered in a controlled setting, has frequently been reported to cause transient, delayed headache, with incidence, duration, and severity increased in a dose-related manner [Johnson et al., Drug Alcohol Depend 2012, 123, 132-140]. Cardiovascular effects, including vasoconstriction and increases in blood pressure and heart rate, have also been reported following administration of classical psychedelics that activate the 5-HT2A receptor. Many of the existing psychedelics are also modulators of other monoamine receptors as secondary targets in addition to the 5-HT2A receptor, and in many cases, it remains unclear how those secondary targets contribute to the efficacy and safety of such compounds. For example, many psychedelics are agonists of the 5-HT2B receptor, activation of which has been linked to cardiac valvulopathy. Others activate the 5-HT1A receptor, activation of which is also known to lead to effects on mood and therefore, may also contribute to the efficacy of such agents. Accordingly, there remains a need for novel psychedelics that possess primary and secondary pharmacology better optimized for the treatment of psychiatric disorders with minimal side effects.

Further, given the profound hallucinations induced by psychedelic drugs, psychedelic therapy is most safely delivered in a supervised medical setting. This comes with significant inconvenience and expense for the patient and health care providers, especially considering the extended duration of effect (4 h+) of the most commonly used agents (e.g., psilocybin). Accordingly, there remains a need for novel psychedelic drugs of shorter duration of action to facilitate more efficient supervised therapy or with hallucinogenic effects that are sufficiently attenuated to make them safely useable at home, away from medical supervision.

The present disclosure relates to a novel class of psychedelic drugs with improved properties.

### SUMMARY OF THE DISCLOSURE

The present disclosure relates to compounds of Formula I

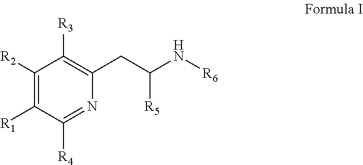

or pharmaceutically acceptable salts thereof,
wherein
R₁ is hydrogen, C₁-C₈ alkyl, C₂-C₈ alkenyl, C₂-C₈ alkynyl, C₃-C₁₀ cycloalkyl, 3- to 6-membered heterocyclyl, C₃-C₁₀ cycloalkyl C₁-C₈ alkyl, 3- to 6-membered heterocyclyl C₁-C₈ alkyl, —OR₇, —SR₇, halo, —CF₃, —SF₅, —OCF₃, —CN, —NO₂, —NR₈R₉, aryl, heteroaryl, aryl C₁-C₈ alkyl, or heteroaryl C₁-C₈ alkyl;
R₂ is hydrogen, C₁-C₈ alkyl, C₂-C₈ alkenyl, C₂-C₈ alkynyl, C₃-C₁₀ cycloalkyl, C₃-C₁₀ cycloalkyl C₁-C₈ alkyl, —OR₁₀, —SR₁₀, halo, —CF₃, —SF₅, —OCF₃, —CN, —NO₂, or —NR₁₁R₁₂;
R₃ is hydrogen, C₁-C₈ alkyl, C₂-C₈ alkenyl, C₂-C₈ alkynyl, C₃-C₁₀ cycloalkyl, C₃-C₁₀ cycloalkyl C₁-C₈ alkyl, —OR₁₃, —SR₁₃, halo, —CF₃, —SF₅, —OCF₃, —CN, —NO₂, —NR₁₄R₁₅, —C(O)R₁₆, —C(O)OR₁₆, —O—C(O)R₁₆, or —C(O)NR₁₇R₁₈;
R₄ is hydrogen, C₁-C₈ alkyl, C₂-C₈ alkenyl, C₂-C₈ alkynyl, C₃-C₁₀ cycloalkyl, C₃-C₁₀ cycloalkyl C₁-C₈ alkyl, —OR₁₉, —SR₁₉, halo, —CF₃, —SF₅, —OCF₃, —CN, —NO₂, —NR₂₀R₂₁, —C(O)R₂₂, —C(O)OR₂₂, —O—C(O)R₂₂, or —C(O)NR₂₃R₂₄;
R₅ is hydrogen, C₁-C₃ alkyl, C₂-C₃ alkenyl, C₂-C₃ alkynyl, C₁-C₃ alkoxy, cyclopropyl, or cyclopropylmethyl; and
R₆ is hydrogen or benzyl, wherein the phenyl ring of benzyl is optionally substituted with 1-5 instances of R₆ₐ and each R₆ₐ is independently selected for each occurrence from the group consisting of hydroxyl, C₁-C₅ alkyl, C₂-C₅ alkenyl, C₂-C₅ alkynyl, C₃-C₁₀cycloalkyl, C₃-C₁₀ cycloalkyl C₁-C₈ alkyl, halo, —CF₃, —SF₅, —OCF₃, —OR₂₅, —SR₂₅, —CN, —NO₂, —NR₂₆R₂₇, —C(O)R₂₈, —C(O)OR₂₈, —O—C(O)R₂₈, and —C(O)NR₂₉R₃₀, or wherein any two adjacent R₆ₐ can be taken together with the atoms on which they are attached to form a C₄-C₆ cycloalkyl or 4- to 6-membered heterocyclyl;
wherein
R₇ is hydrogen, C₁-C₈ alkyl, C₂-C₈ alkenyl, C₂-C₈ alkynyl, C₃-C₁₀ cycloalkyl, C₃-C₁₀ cycloalkyl C₁-C₈ alkyl, aryl, heteroaryl, aryl C₁-C₈ alkyl, heteroaryl C₁-C₈ alkyl, 3- to 6-membered heterocyclyl, or 3- to 6-membered heterocyclyl C₁-C₈ alkyl;
R₈ and R₉ are independently hydrogen, C₁-C₈alkyl, C₂-C₈ alkenyl, C₂-C₈ alkynyl, C₃-C₁₀ cycloalkyl, C₃-C₁₀ cycloalkyl C₁-C₈alkyl, aryl, aryl C₁-C₈alkyl, heteroaryl, heteroaryl C₁-C₈ heteroaryl, 3- to 6-membered heterocyclyl, or 3- to 6-membered heterocyclyl C₁-C₈alkyl;
R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, and R₃₀ are independently hydrogen, C₁-C₅ alkyl, C₂-C₅ alkenyl, C₂-C₅ alkynyl, C₃-C₆ cycloalkyl, C₃-C₆ cycloalkyl C₁-C₅ alkyl, 3- to 6-membered heterocyclyl, or 3- to 6-membered heterocyclyl C₁-C₅ alkyl;
   aryl is a monocyclic or bicyclic aromatic ring containing 6 or 10 ring carbon atoms;
heteroaryl is a 5- to 10-membered ring, which is either monocyclic or bicyclic, containing 1, 2, 3, or 4 ring heteroatoms and 2 to 9 ring carbon atoms;
aryl and heteroaryl groups may be unsubstituted or substituted with one or more substituents independently selected from hydroxyl, C₁-C₅ alkyl, C₂-C₅ alkenyl, C₂₋₅ alkynyl, C₃-C₆ cycloalkyl, C₁-C₅ alkoxy, halo, —CF₃, —CN, —NO₂, —NH₂, aryl, heteroaryl, C₃-C₆cycloalkyl C₁-C₃ alkyl, aryl C₁-C₃ alkyl, heteroaryl C₁-C₃ alkyl, 3- to 6-membered heterocyclyl, and 3- to 6-membered heterocyclyl C₁-C₃ alkyl;
cycloalkyl and heterocyclyl groups may be unsubstituted or substituted with one or more substituents independently selected from hydroxyl, C₁-C₅ alkyl, C₂-C₅ alkenyl, C₂-C₅ alkynyl, C₃-C₆ cycloalkyl, C₁-C₅ alkoxy, halo, —CF₃, —CN, —NO₂, —NH₂, aryl, heteroaryl, C₃-C₆ cycloalkyl C₁-C₃ alkyl, aryl C₁-C₃ alkyl, heteroaryl C₁-C₃ alkyl, 3- to 6-membered heterocyclyl, and 3- to 6-membered heterocyclyl C₁-C₃ alkyl; and
alkyl, alkenyl, and alkynyl groups may be unsubstituted or substituted with one or more substituents independently selected from hydroxyl, C₃-C₆ cycloalkyl. C₁-C₅ alkoxy, halo, —CF₃, —CN, —NO₂, —NH₂, aryl, heteroaryl, C₃-C₆ cycloalkyl C₁-C₃ alkyl, aryl C₁-C₃ alkyl, heteroaryl C₁-C₃alkyl, 3- to 6-membered heterocyclyl, and 3- to 6-membered heterocyclyl C₁-C₃ alkyl.

These compounds are useful for treating psychiatric disorders.

### DETAILED DESCRIPTION

Unless otherwise indicated, the definitions and embodiments described in this and other sections are intended to be applicable to all embodiments and aspects of the present disclosure herein described for which they are suitable as would be understood by a person skilled in the art.

All features disclosed in the specification, including the claims, abstract, and all the steps in any method or process disclosed, may be combined in any combination, except combinations where at least some of such features and/or steps are mutually exclusive. Each feature disclosed in the specification, including the claims and abstract, can be replaced by alternative features serving the same, equivalent, or similar purpose, unless expressly stated otherwise.

The term “compound(s) of the disclosure” or “compound(s) of the present disclosure” and the like, as used herein, refers to a compound of Formula I or pharmaceutically acceptable salts thereof.

The term “composition(s) of the disclosure” or “composition(s) of the present disclosure” and the like, as used herein, refers to a composition, such as a pharmaceutical composition, comprising one or more compounds of the disclosure.

The term “and/or” as used herein means that the listed items are present, or used, individually or in combination. In effect, this term means that “at least one of” or “one or more” of the listed items is used or present. The term “and/or” with respect to pharmaceutically acceptable salts, means that the compounds of the disclosure exist as individual salts and solvates, as well as a combination of, for example, salts of a compound of the disclosure.

As used in the present disclosure, the singular forms “a”, “an” and “the” include plural references unless the content clearly dictates otherwise. For example, an embodiment including “a compound” should be understood to present certain aspects with one compound, or two or more additional compounds.

As used in this disclosure and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps. A subset of comprising is “consisting essentially of,” which is defined hereinbelow, and a subset of “consisting essentially of” is “consisting of,” which is defined hereinbelow. Thus, it is understood whenever the term comprising is used, it may be replaced with “consisting essentially of” or “consisting of” and these embodiments are contemplated within the present disclosure.

The term “consisting of” and its derivatives as used herein are intended to be closed terms that specify the presence of the stated features, elements, components, groups, integers, and/or steps and also exclude the presence of other unstated features, elements, components, groups, integers, and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps. A subset of “consisting essentially of” is “consisting of 3- to 6-membered heterocyclyl, or 3- to 6-membered heterocyclyl, C₁-C₈ alkyl,” which is defined hereinabove. Thus, it is understood whenever the term “consisting essentially of” is used, it may be replaced with “consisting of” and these embodiments are contemplated within the present disclosure.

The term “suitable” as used herein with respect to a synthetic pathway means that the selection of the particular compound or conditions would depend on the specific synthetic manipulation to be performed, the identity of the molecule(s) to be transformed, and/or the specific use for the compound, but the selection would be well within the skill of a person trained in the art. All process/method steps described herein are to be conducted under conditions effective to provide the product shown. A person skilled in the art would understand that all reaction conditions, including, for example, reaction solvent, reaction time, reaction temperature, reaction pressure, reactant ratio, and whether or not the reaction should be performed under an anhydrous or inert atmosphere, can be varied to optimize the yield of the desired product and it is within their skill to do so.

The terms “about” or “approximately” as used herein mean within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, a range up to 10%, a range up to 5%, and/or a range up to 1% of a given value. Alternatively, particularly with respect to biological systems or processes, the term can mean within an order of magnitude, e.g., within 10-fold, or within 3-fold, of a value. “About” and “approximately” are synonymous and are used interchangeably herein.

The term “substantially”, as used herein, means a reasonable amount of deviation of the modified term such that the end result is not significantly changed. These terms of degree should be construed as including a deviation of at least ±0.5% of the modified term if this deviation would not negate the meaning of the word it modifies, unless the context suggests otherwise to a person skilled in the art. As an example, in an embodiment, the term “substantially” means about 95% of a given value. For instance, the term “substantially all” signifies all or at least 95% of all members of a given set.

The present description refers to several chemical terms and abbreviations used by those skilled in the art. Nevertheless, definitions of selected terms are provided for clarity and consistency.

The term “alkyl”, as used herein, whether it is used alone or as part of another group, means straight or branched chain, saturated hydrocarbon groups (alkyl groups). The number of carbon atoms that are possible in the referenced alkyl group are indicated by the prefix “Cₙ₁₋ₙ₂” or “Cₙ₁-Cₙ₂”, wherein n₁ and n₂ are independently integers ranging from 1 to 8, inclusive. Thus, for example, the term “C₁₋₆ alkyl” or “C₁-C₆ alkyl” means an alkyl group having 1, 2, 3, 4, 5, or 6 carbon atoms and includes, for example, any of the hexyl alkyl and pentyl alkyl isomers as well as n-, iso-, sec- and tert-butyl, n- and iso-propyl, ethyl, and methyl. As another example, “C₁₋₄ alkyl” refers to n-, iso-, sec- and tert-butyl, n- and isopropyl, ethyl, and methyl. The alkyl group may be unsubstituted or substituted, and the substituents thereon are as described hereinabove, to the extent such substitution makes chemical sense.

The term “alkenyl” as used herein, whether it is used alone or as part of another group, means straight or branched chain, unsaturated hydrocarbon groups containing at least one carbon-carbon double bond and containing up to four carbon-carbon double bonds. The number of carbon atoms that are possible in the referenced alkenyl group are indicated by the prefix “Cₙ₃₋ₙ₄” or “Cₙ₃-Cₙ₄”, wherein n₃ and n₄ are independently integers ranging from 2 to 8, inclusive. Thus, for example, the term “C₂₋₈ alkenyl” or “C₂-C₈ alkenyl” means an alkenyl group containing 2-8 carbon atoms. Examples include ethenyl, 1-propenyl, 2-propenyl, 1-butenyl, 2-butenyl, 1-pentenyl, 2-pentenyl, 1-hexenyl, 2-hexenyl, 3-hexenyl 1,3-butadienyl, 1,3-pentadienyl, 1,4-pentadienyl, 2,4-pentadienyl, 1-hexenyl, 2-hexenyl, 3-hexenyl, 1,3-hexadienyl, 1,4-hexadienyl, 1,5-hexadienyl, 2-4-hexadieny, 2,5-hexadienyl, 1,3,5-hexatrienyl, 1-heptene, 2-heptene, 3-heptene, 1-octene, 2-octene, 3-octene, 4-octene, 1,3,5,7-octatetraene, and the like. The alkenyl group may be unsubstituted or substituted, and the substituents thereon are as described hereinabove, to the extent such substitution makes chemical sense.

The term “alkynyl” as used herein, whether it is used alone or as part of another group, means straight or branched chain, unsaturated hydrocarbon groups containing at least one carbon-carbon triple bond and containing up to four carbon-carbon triple bonds. The number of carbon atoms that are possible in the referenced alkynyl group are indicated by the prefix “Cₙ₅₋ₙ₆” or “Cₙ₅-Cₙ₆”, wherein n₅ and n₆ are independently integers ranging from 2 to 8, inclusive. Thus, for example, the term “C₂₋₈ alkynyl” or “C₂-C₈ alkynyl” means an alkynyl group containing 2-8 carbon atoms. Examples include 1-ethynyl, 1-propynyl, 1-butynyl, 2-butynyl, 1-pentynyl, 1-hexynyl, 1,5-hexadiynyl, 1-heptynyl, 1-octynyl, 1,3,5-heptatriynyl, and the like. The alkynyl group may be unsubstituted or substituted, and the substituents thereon are as described hereinabove, to the extent such substitution makes chemical sense.

The terms “cycloalkyl” and “cyclyl” are synonymous, and as used herein, whether used alone or as part of another group, mean a saturated carbocyclic group containing at least three ring carbon atoms and one or more rings. The number of carbon ring atoms that are possible in the referenced cycloalkyl group are indicated by the numerical prefix “Cₙ₇₋ₙ₈ cycloalkyl”, wherein n₇ and n₈ are independently integers ranging from 3 to 10, inclusive. For example, the term C₃₋₁₀ cycloalkyl means a cycloalkyl group having 3, 4, 5, 6, 7, 8, 9, or 10 ring carbon atoms and either one or two fused rings, and the term C₃-C₆ cycloalkyl refers to a cycloalkyl group having 3, 4, 5, or 6 ring carbon atoms. However, the term includes cycloalkyl groups that are substituted with up to 20 carbon atoms, wherein each of the substituents themselves are alkyl groups having 1-10 carbon atoms. Examples of cycloalkyl include cyclopropyl, cyclobutyl, cyclopentyl, cyclohexyl, cyclohexyl, 2,3-dimethylcyclohexyl, 2-ethylcyclopentyl, and the like. The term “cycloalkyl”, as used herein, also includes bicyclic ring systems in which one or both rings are cycloalkyl, e.g., decalinyl. The cycloalkyl group may be unsubstituted or substituted, and the substituents thereon are as described hereinabove, to the extent such substitution makes chemical sense.

The term cycloalkenyl, as used herein, refers to a cyclic unsaturated hydrocarbon, excluding aromatics, which contains 3 to 10 ring carbon atoms and one or more rings and at least one carbon-carbon double bond. It may contain 1, 2, 3, or 4 carbon-carbon double bonds. The number of carbon ring atoms that are possible in the referenced cycloalkenyl group are indicated by the numerical prefix “Cₙ₉₋ₙ₁₀ cycloalkenyl”, wherein n₉ and n₁₀ are independently integers ranging from 4 to 10, inclusive. Examples include cyclobutene, cyclopentene, cyclohexene, cycloheptene, 1,3-cyclohexadiene,1,4-cyclohexadiene, and the like. The cycloalkenyl group may be unsubstituted or substituted, and the substituents thereon are as described hereinabove, to the extent such substitution makes chemical sense.

The terms “aryl” and “aromatic” are synonymous, and as used herein, whether used alone or as part of another group, refer to carbocyclic groups containing at least one aromatic ring, 6 to 20 carbon atoms, and 6, 10, 14, and 18 ring carbon atoms. As used herein, this term excludes heteroaryl. The aryl group may be monocyclic, bicyclic, tricyclic, or tetracyclic. The aromatic ring may be substituted, e.g., by alkyl groups. The aromatic ring may be fused to a cycloalkyl, and for purposes of this disclosure, is considered an aromatic group. Examples of aryl include phenyl, α-naphthyl, β-naphthyl, anthracenyl, azulenyl, phenanthrenyl, indanyl, indenyl, tolyl, xylyl, fluorenyl, and the like. The aryl group may be unsubstituted or substituted, and the substituents thereon are as described hereinabove, to the extent such substitution makes chemical sense.

The terms “heterocyclyl” or “heterocyclic” may be used interchangeably to refer to a non-aromatic, saturated or partially unsaturated ring system, containing the specified number of ring atoms, including at least one ring heteroatom selected from N, O, and S as a ring member, where ring S atoms are optionally substituted by one or two oxo groups (i.e., S(O)x, where x is 0, 1 or 2), and where the heterocyclic ring is connected to the base molecule via a ring atom, which may be C or N, and wherein the heterocyclic ring does not contain two contiguous oxygen atoms. Unless indicated to the contrary, the heterocyclyl group contains 3 to 10 ring atoms and 1 to 9 ring carbon atoms. In an embodiment, the heterocyclyl contains 1-4 ring heteroatoms, as defined herein. In an embodiment, the heterocyclic ring contains 5 to 10 ring atoms and 2 to 9 ring carbon atoms. Heterocyclic rings include rings which are spirocyclic, bridged, or fused to one or more other heterocyclic or carbocyclic rings, where such spirocyclic, bridged, or fused rings may themselves be saturated, partially unsaturated or aromatic to the extent unsaturation or aromaticity makes chemical sense, provided the point of attachment to the base molecule is an atom of the heterocyclic portion of the ring system. Examples of heterocyclic groups include, but are not limited to, azetidinyl, pyrrolidinyl, piperidinyl, piperazinyl, azepanyl, diazepanyl, oxetanyl, tetrahydrofuranyl, tetrahydropyranyl, tetrahydrothiopyranyl, morpholinyl, and thiomorpholinyl, each of which are optionally substituted as described for the particular substituent group, to the extent such substitution makes chemical sense.

The term “3- to 6-membered heterocyclyl”, as used herein, refers to a heterocyclic group containing 3, 4, 5, or 6 ring atoms, wherein at least one of the ring atoms is a heteroatom selected from N, O, or S, and containing 1-5 carbon atoms, with no contiguous ring oxygen atoms. A “4- to 6-membered heterocyclyl”, as used herein, refers to a heterocyclic group containing 4, 5, or 6 ring atoms, wherein at least one of the ring atoms is a heteroatom selected from N, O, or S, and containing 1-5 carbon atoms, with no contiguous ring oxygen atoms. Thus, for example, a 3-membered heterocyclyl contains either 1 or 2 ring carbon atoms, a 4-membered heterocyclyl contains 1, 2, or 3 ring carbon atoms, a 5-membered heterocyclyl contains 1, 2, 3, or 4 ring carbon atoms, and a 6-membered heterocyclyl contains 1, 2, 3, 4, or 5 ring carbon atoms. These heterocyclyl may be unsubstituted or substituted. Unless indicated otherwise, the substituents are as defined hereinbelow for heterocyclyl.

Similarly, “heteroaryl” or “heteroaromatic” refer to monocyclic or fused bicyclic ring systems containing 1, 2, 3 or 4 ring heteroatoms and 2 to 9 ring carbon atoms having the well-known characteristics of aromaticity that contain the specified number of ring atoms and include at least one heteroatom selected from N, O, and S as a ring member in an aromatic ring. Heteroaryl rings are attached to the base molecule via a ring atom of the heteroaromatic ring, such that aromaticity is maintained. Thus, 6-membered heteroaryl rings may be attached to the base molecule via a ring C atom, while 5-membered heteroaryl rings may be attached to the base molecule via a ring C or N atom. Heteroaryl groups may also be fused to another aryl or heteroaryl ring or fused to a saturated or partially unsaturated carbocyclic or heterocyclic ring, provided the point of attachment to the base molecule on such fused ring systems is an atom of the heteroaromatic portion of the ring system. It is understood that no more than two N, O, or S atoms are ordinarily connected sequentially, except where an oxo group is attached to N or S to form a nitro or sulfonyl group, or in the case of certain heteroaromatic rings, such as triazine, triazole, tetrazole, oxadiazole, thiadiazole, and the like. Examples of unsubstituted heteroaryl often includes, but are not limited to, pyrrole, furan, thiophene, pyrazole, imidazole, isoxazole, oxazole, isothiazole, thiazole, triazole, oxadiazole, thiadiazole, tetrazole, pyridine, pyridazine, pyrimidine, pyrazine, benzofuran, benzothiophene, indole, benzimidazole, indazole, quinoline, isoquinoline, purine, triazine, naphthyridine, carbazole, and the like. The heteroaryl group may be unsubstituted or substituted, and the substituents thereon are as described hereinabove, to the extent such substitution makes chemical sense.

The terms “heterocyclic Cₙ₁₉-Cₙ₂₀ alkyl” and “heterocyclyl Cₙ₁₉-Cₙ₂₀ alkyl” are used interchangeably and are synonymous, and may be used to describe a heterocyclic group, as defined herein, that is connected to the base molecule through an alkylene linker of the specified length of n₁₉ to n₂₀ carbon atoms, wherein n₁₉ and n₂₀ are independently 1, 2, 3, 4, 5, 6, 7, or 8.

For example, such groups contain an optionally substituted heterocyclic attached to the base molecule through a Cₙ₁₉-Cₙ₂₀ alkylene linker, such as a C₁-C₈, C₁-C₅, or C₁-C₃ linker. Where so indicated, such groups are optionally substituted on the alkylene portion by the same groups that are described herein as suitable for alkyl groups and on the heterocyclic portion by groups described as suitable for heterocyclic rings.

The term “heteroaryl Cₙ₁₇-Cₙ₁₈ alkyl”, which is synonymous with “Cₙ₁₇-Cₙ₁₈ alkyl heteroaryl”, as these terms are used herein, describes a heteroaryl group, as defined herein, that is connected to the base molecule through an alkylene linker of the specified length of n₁₇ to n₁₈ carbon atoms, wherein n₁₇ and n₁₈ independently are 1, 2, 3, 4, 5, 6, 7, or 8. For example, such groups contain an optionally substituted heteroaryl attached to the base molecule through a Cₙ₁₇-Cₙ₁₈ alkylene linker, such as a C₁-C₈, C₁-C₅, or C₁-C₃ linker. Where so indicated, such groups are optionally substituted on the alkylene portion by the same groups that are described herein as suitable for alkyl groups and on the heteroaryl portion by groups described as suitable for heteroaryl rings.

The term “cycloalkyl Cₙ₁₅-Cₙ₁₆ alkyl” may be used to describe a cycloalkyl group, as defined herein, that is connected to the base molecule through an alkylene linker of the specified length of n₁₅ to n₁₆ carbon atoms, wherein n₁₅ and n₁₆ are independently 1, 2, 3, 4, 5, 6, 7, or 8. For example, such groups contain an optionally substituted cycloalkyl attached to the base molecule through a Cₙ₁₅-Cₙ₁₆ alkylene linker, such as a C₁-C₈, C₁-C₅, or C₁-C₃ linker. Where so indicated, such groups are optionally substituted on the alkylene portion by the same groups that are described herein as suitable for alkyl groups and on the cycloalkyl portion by groups described as suitable for cycloalkyl rings. Examples of such groups include, but are not limited to, cyclopropylmethyl, 2-cyclopropylethyl, 2-cyclopentylethyl, cyclohexyl-4-pentyl, and the like.

The term “aryl Cₙ₁₃-Cₙ₁₄ alkyl” may be used to describe an aryl group, as defined herein, of the specified size that is connected to the base molecule through an alkylene linker of the specified length of n₁₃ or n₁₄ carbon atoms, wherein n₁₃ and n₁₄ are independently 1, 2, 3, 4, 5, 6, 7, or 8. For example, such groups contain an optionally substituted aryl group attached to the base molecule through a Cₙ₁₃-Cₙ₁₄ alkylene linker, such as a C₁-C₈, C₁-C₅, or C₁-C₃ linker. Where so indicated, such groups are optionally substituted on the alkylene portion by the same groups that are described herein as suitable for alkyl groups and on the aryl portion by groups described as suitable for aryl rings. Examples include benzyl, phenethyl, phen-2-propyl, naphthylmethyl, and the like.

The term “halogen” (or “halo”), whether it is used alone or as part of another group, refers to a halogen atom and includes fluoro, chloro, bromo, and iodo.

As used herein, the term “alkoxy”, alone or in combination, refers to an alkyl group, as defined above, connected to an oxygen connecting atom. Examples include, but are not limited to, methoxy, ethoxy, propoxy, isopropoxy, n-butoxy, 2-methyl-1-propoxy, and the like.

The term “Cₙ₁₁-Cₙ₁₂ alkylthio”, as used herein, alone or in combination, refers to an alkyl group, as defined above, wherein n₁₁ and n₁₂ are independently 1, 2, 3, 4, 5, 6, 7, or 8, connected to a sulfur connecting atom, which is in turn connected to the base molecule. Examples include, but are not limited to, methylthio, ethylthio, and the like.

The term “hydroxyalkyl” as used herein, alone or in combination, refers to an alkyl group, as defined above, substituted with one or more hydroxyl groups. Examples include, but are not limited to, —CH₂—OH, —CH₂—CH₂—OH, —CH₂—CH(OH)—CH₃, and the like.

With respect to R₆, the term “wherein any two adjacent R₆ₐ can be taken together with the atoms on which they are attached to form a C₄-C₆ cycloalkyl or 4- to 6-membered heterocyclyl” indicates that the aryl ring in benzyl is fused to either C₄-C₆ cycloalkyl or 4- to 6-membered heterocyclyl ring.

The term “available”, as in “available hydrogen atoms” or “available atoms” refers to atoms that would be known to a person skilled in the art to be capable of replacement by a substituent.

The term “pharmaceutically acceptable” means compatible with the treatment of subjects.

The term “pharmaceutically acceptable carrier” means a non-toxic solvent, dispersant, excipient, adjuvant, or other material which is mixed with the active ingredient to permit the formation of a pharmaceutical composition, i.e., a dosage form capable of administration to a subject.

The term “pharmaceutically acceptable salt” means either an acid addition salt or basic addition salt which is suitable for, or compatible with, the treatment of subjects.

An acid addition salt of the compound of Formula I is a salt of the amine functionality by the addition of an acid. An acid addition salt suitable for, or compatible with, the treatment of subjects is any non-toxic organic or inorganic acid addition salt of any basic compound. Illustrative inorganic acids which form suitable salts include hydrochloric, hydrobromic, sulfuric, nitric, and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxy maleic, benzoic, hydroxybenzoic, phenylacetic, cinnamic, mandelic, salicylic, 2-phenoxybenzoic, p-toluenesulfonic acid, and other sulfonic acids, such as methanesulfonic acid, ethanesulfonic acid, and 2-hydroxyethanesulfonic acid. In some embodiments, exemplary acid addition salts also include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (“mesylates”), naphthalenesulfonates, nitrates, oxalates, phosphates, propionates, salicylates, succinates, sulfates, tartarates, thiocyanates, toluenesulfonates (also known as tosylates), and the like. In some embodiments, the mono- or di-acid salts are formed, and such salts exist in either a hydrated, solvated, or substantially anhydrous form. In general, acid addition salts are more soluble in water and various hydrophilic organic solvents and generally demonstrate higher melting points in comparison to their free base forms. The selection criteria for the appropriate salt will be known to one skilled in the art. Other non-pharmaceutically acceptable salts, such as but not limited to picrates may be used, for example in the isolation of compounds of the disclosure for laboratory use, or for subsequent conversion to a pharmaceutically acceptable acid addition salt. Methods of making such salts will be well-known to those skilled in the art. The salt may be prepared, for example, by adding the free base compound to a solution of the counter-ion in a solvent. The solvent may be a polar, protic solvent, such as 2-propanol or methanol, and may include a polar, aprotic cosolvent, such as dichloromethane. Salts may be precipitated or crystallized by the addition of a counter solvent in which the salt is less soluble, for example, diethyl ether or ethyl acetate.

A basic addition salt suitable for, or compatible with, the treatment of subjects is any non-toxic organic or inorganic base addition salt of any acidic compound. Acidic compounds that form a basic addition salt include, for example, compounds comprising a carboxylic acid group or phenol group. The selection criteria for the appropriate salt will be known to one skilled in the art. In some embodiments, exemplary basic salts also include ammonium salts, alkali metal salts such as sodium, lithium, and potassium salts, and alkaline earth metal salts such as calcium and magnesium salts, Illustrative inorganic bases which form suitable salts include lithium, sodium, potassium, calcium, magnesium, or barium hydroxide as well as ammonia. Illustrative organic bases which form suitable salts include aliphatic, alicyclic, or aromatic organic amines such as isopropylamine, methylamine, trimethylamine, picoline, diethylamine, triethylamine, tripropylamine, ethanolamine, 2-dimethylaminoethanol, 2-diethylaminoethanol, cyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like.

Basic nitrogen containing groups may be quarternized with agents such as lower alkyl halides (e.g., methyl, ethyl and butyl chlorides, bromides, and iodides), di(lower) alkyl sulfates (e.g., dimethyl, diethyl and dibutyl sulfates), long chain halides, such as C₁₀₋₂₀, hydrocarbyl halides (e.g., decyl, lauryl and stearyl chlorides, bromides, and iodides), arylalkyl halides (e.g., benzyl and phenethyl bromides) and others, wherein lower alkyl is defined as C1-C6 alkyl. Compounds carrying an acidic moiety can be mixed with suitable pharmaceutically acceptable salts to provide, for example, alkali metal salts (e.g., sodium or potassium salts), alkaline earth metal salts (e.g., calcium or magnesium salts), and salts formed with suitable organic ligands such as quaternary ammonium salts. Also, in the case of an acid (—COOH) or phenolic group being present, pharmaceutically acceptable esters can be employed to modify the solubility or hydrolysis characteristics of the compound.

All such acid salts and base salts are intended to be pharmaceutically acceptable salts within the scope of the application and all acid and base salts are considered equivalent to the free forms of the corresponding compounds for purposes of the application. In addition, when a compound of the application contains both a basic moiety, and an acidic moiety, zwitterions (“inner salts”) may be formed and are included within the term “salt(s)” as used herein. It is understood that certain compounds of the application may exist in zwitterionic form, having both anionic and cationic centers within the same compound and a net neutral charge. Such zwitterions are included within the scope of the present disclosure.

The term “solvate” as used herein means a compound, or a salt or prodrug of a compound, wherein molecules of a suitable solvent are incorporated in the crystal lattice. A suitable solvent is physiologically tolerable at the dosage administered.

The term “prodrug” as used herein means a compound, or salt of a compound, that, after administration, is converted into an active drug.

The term “protecting group” or “PG” and the like, as used herein, refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3.sup.1d Edition, 1999 and in Kocienski, P. Protecting Groups, 3rd Edition, 2003, Georg Thieme Verlag (The Americas).

The term “subject”, as used herein, includes all members of the animal kingdom including mammals, and suitably refers to humans. Thus, the methods of the present disclosure are applicable to both human therapy and veterinary applications.

The term “treating” or “treatment”, as used herein, and as is well understood in the art, means an approach for obtaining beneficial or desired results, including clinical results. Beneficial or desired clinical results include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment”, as used herein, also include prophylactic treatment to retard further progression of the disease. For example, a subject with early psychiatric illness can be treated to prevent progression, or alternatively a subject in remission can be treated with a compound or composition of the disclosure to prevent recurrence. Treatment methods comprise administering to a subject a therapeutically effective amount of one or more of the compounds of the disclosure and optionally consist of a single administration, or iteratively comprise a series of administrations.

As used herein, the term “effective amount” or “therapeutically effective amount” means an amount of one or more compounds of the disclosure that is effective, at dosages and for periods of time necessary to achieve the desired result. For example, in the context of treating a disease, disorder, or condition mediated or treated by agonism or activation of serotonin receptors and downstream second messengers, an effective amount is an amount that, for example, increases said activation compared to the activation without administration of the one or more compounds. As an example, the terms “effective amount” or “therapeutically effective amount” refer to an amount of a compound, material, composition, medicament, or other material that is effective to achieve a particular pharmacological and/or physiologic effect including, but not limited to, reducing the frequency or severity of sadness or lethargy, depressed mood, anxious or sad feelings, diminished interest in all or nearly all activities, significant increased or decreased appetite leading to weight gain or weight loss, insomnia, irritability, fatigue, feelings of worthlessness, feelings of helplessness, inability to concentrate, and recurrent thoughts of death or suicide, or to provide a desired pharmacologic and/or physiologic effect, for example, reducing, inhibiting, or reversing one or more of the underlying pathophysiological mechanisms underlying the neurological dysfunction, modulating dopamine levels or signaling, modulating serotonin levels or signaling, modulating norepinephrine levels or signaling, modulating glutamate or GABA levels or signaling, modulating synaptic connectivity or neurogenesis in certain brain regions, or a combination thereof.

The term “therapeutic index” used in reference to any compound and its associated therapeutic effects and side effects refers to the ratio of the dose of said compound required to induce a particular negative side effect to the dose of said compound required to induce the desired therapeutic effect. For example, in some embodiments, the antidepressant therapeutic effects and hallucinogenic side effects occur at similar doses and thus, the therapeutic index of this compound in this context is about 1:1. In contrast, a compound disclosed herein might have an improved therapeutic index, for example 3:1, where a 3-fold higher dose is required to induce hallucinogenic side effects relative to that needed for antidepressant therapeutic effects.

“Palliating” a disease, disorder or condition means that the extent and/or undesirable clinical manifestations of a disease, disorder, or condition are lessened and/or the time course of the progression is slowed or lengthened, as compared to not treating the disorder.

The term “administered”, as used herein, means administration of a therapeutically effective amount of one or more compounds or compositions of the disclosure to a cell, tissue, organ, or subject.

The term “prevention” or “prophylaxis”, or synonym thereto, as used herein, refers to a reduction in the risk or probability of a patient becoming afflicted with a disease, disorder, or condition, or manifesting a symptom associated with a disease, disorder, or condition.

The terms “disease”, “disorder”, or “condition”, as used herein, refer to a disease, disorder, or condition treated or treatable by activation of a serotonin receptor, for example, the 5-HT2A receptor, and particularly, using a serotonin receptor agonist, such as one or more of the compounds herein described.

The term “treating a disease, disorder, or condition by activation of a serotonin receptor”, as used herein, means that the disease, disorder, or condition to be treated is affected by, modulated by, and/or has some biological basis, either direct or indirect, that includes serotonergic activity. These diseases respond favorably when serotonergic activity associated with the disease, disorder, or condition is agonized by one or more of the compounds or compositions of the disclosure.

The term “activation”, as used herein, includes agonism, partial agonism, and positive allosteric modulation, of a serotonin receptor.

The term “5-HT2A”, as used herein, means the 5-HT2A serotonin receptor.

The present disclosure includes a compound of Formula I or a pharmaceutically acceptable salt thereof:

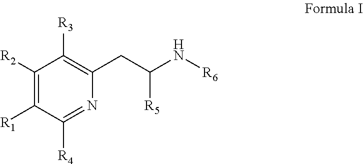

wherein
or pharmaceutically acceptable salts thereof,
wherein
R₁ is hydrogen, C₁-C₈ alkyl, C₂-C₈ alkenyl, C₂-C₈ alkynyl, C₃-C₁₀ cycloalkyl, 3- to 6-membered heterocyclyl, C₃-C₁₀ cycloalkyl C₁-C₈ alkyl, 3- to 6-membered heterocyclyl C₁-C₈ alkyl, —OR₇, —SR₇, halo, —CF₃, —SF₅, —OCF₃, —CN, —NO₂, —NR₈R₉, aryl, heteroaryl, aryl C₁-C₈ alkyl, or heteroaryl C₁-C₈alkyl;
R₂ is hydrogen, C₁-C₈ alkyl, C₂-C₈ alkenyl, C₂-C₈ alkynyl, C₃-C₁₀ cycloalkyl, C₃-C₁₀ cycloalkyl C₁-C₈ alkyl, —OR₁₀, —SR₁₀, halo, —CF₃, —SF₅, —OCF₃, —CN, —NO₂, or —NR₁₁R₁₂;
R₃ is hydrogen, C₁-C₈ alkyl, C₂-C₈ alkenyl, C₂-C₈ alkynyl, C₃-C₁₀ cycloalkyl, C₃-C₁₀ cycloalkyl C₁-C₈ alkyl, —OR₁₃, —SR₁₃, halo, —CF₃, —SF₅, —OCF₃, —CN, —NO₂, —NR₁₄R₁₅, —C(O)R₁₆, —C(O)OR₁₆, —O—C(O)R₁₆, or —C(O)NR₁₇R₁₈;
R₄ is hydrogen, C₁-C₅ alkyl, C₂-C₅ alkenyl, C₂-C₅ alkynyl, C₃-C₁₀ cycloalkyl, C₃-C₁₀ cycloalkyl C₁-C₈ alkyl, —OR₁₉, —SR₁₉, halo, —CF₃, —SF₅, —OCF₃, —CN, —NO₂, —NR₂₀R₂₁, —C(O)R₂₂, —C(O)OR₂₂, —O—C(O)R₂₂, or —C(O)NR₂₃R₂₄;
R₅ is hydrogen, C₁-C₃ alkyl, C₂-C₃ alkenyl, C₂-C₃ alkynyl, C₁-C₃ alkoxy, cyclopropyl, or cyclopropylmethyl; and
R₆ is hydrogen or benzyl, wherein the phenyl ring of benzyl is optionally substituted with 1-5 instances of R₆ₐ and each R₆ₐ is independently selected for each occurrence from the group consisting of hydroxyl, C₁-C₅ alkyl, C₂-C₅ alkenyl, C₂-C₅ alkynyl, C₃-C₁₀cycloalkyl, C₃-C₁₀ cycloalkyl C₁-C₈ alkyl, halo, —CF₃, —SF₅, —OCF₃, —OR₂₅, —SR₂₅, —CN, —NO₂, —NR₂₆R₂₇, —C(O)R₂₈, —C(O)OR₂₈, —O—C(O)R₂₈, and —C(O)NR₂₉R₃₀, or wherein any two adjacent R₆ₐ can be taken together with the atoms on which they are attached to form a C₄-C₆ cycloalkyl or 4- to 6-membered heterocyclyl;
wherein
R₇ is hydrogen, C₁-C₈ alkyl, C₂-C₈ alkenyl, C₂-C₈ alkynyl, C₃-C₁₀ cycloalkyl, C₃-C₁₀ cycloalkyl C₁-C₈ alkyl, aryl, heteroaryl, aryl C₁-C₈ alkyl, heteroaryl C₁-C₈ alkyl, 3- to 6-membered heterocyclyl, or 3- to 6-membered heterocyclyl C₁-C₅ alkyl;
R₈ and R₉ are independently hydrogen, C₁-C₈alkyl, C₂-C₈ alkenyl, C₂-C₈ alkynyl, C₃-C₁₀ cycloalkyl, C₃-C₁₀ cycloalkyl C₁-C₈alkyl, aryl, aryl C₁-C₈alkyl, heteroaryl, heteroaryl C₁-C₈ heteroaryl, 3- to 6-membered heterocyclyl, or 3- to 6-membered heterocyclyl C₁-C₈alkyl;
R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, and R₃₀ are independently hydrogen, C₁-C₅ alkyl, C₂-C₅ alkenyl, C₂-C₅ alkynyl, C₃-C₆ cycloalkyl, C₃-C₆ cycloalkyl C₁-C₅ alkyl, 3- to 6-membered heterocyclyl, or 3- to 6-membered heterocyclyl C₁-C₅ alkyl;
aryl is a monocyclic or bicyclic aromatic ring containing 6 or 10 ring carbon atoms;
heteroaryl is a 5- to 10-membered ring, which is either monocyclic or bicyclic, containing 1, 2, 3, or 4 ring heteroatoms and 2 to 9 ring carbon atoms;
aryl and heteroaryl groups may be unsubstituted or substituted with one or more substituents independently selected from hydroxyl, C₁-C₅ alkyl, C₂-C₅ alkenyl, C₂₋₅ alkynyl, C₃-C₆ cycloalkyl, C₁-C₅ alkoxy, halo, —CF₃, —CN, —NO₂, —NH₂, aryl, heteroaryl, C₃-C₆cycloalkyl C₁-C₃ alkyl, aryl C₁-C₃ alkyl, heteroaryl C₁-C₃ alkyl, 3- to 6-membered heterocyclyl, and 3- to 6-membered heterocyclyl C₁-C₃ alkyl;
cycloalkyl and heterocyclyl groups may be unsubstituted or substituted with one or more substituents independently selected from hydroxyl, C₁-C₅ alkyl, C₂-C₅ alkenyl, C₂-C₅ alkynyl, C₃-C₆ cycloalkyl, C₁-C₅ alkoxy, halo, —CF₃, —CN, —NO₂, —NH₂, aryl, heteroaryl, C₃-C₆ cycloalkyl C₁-C₃ alkyl, aryl C₁-C₃ alkyl, heteroaryl C₁-C₃ alkyl, 3- to 6-membered heterocyclyl, and 3- to 6-membered heterocyclyl C₁-C₃ alkyl; and
alkyl, alkenyl, and alkynyl groups may be unsubstituted or substituted with one or more substituents independently selected from hydroxyl, C₃-C₆ cycloalkyl. C₁-C₅ alkoxy, halo, —CF₃, —CN, —NO₂, —NH₂, aryl, heteroaryl, C₃-C₆ cycloalkyl C₁-C₃ alkyl, aryl C₁-C₃ alkyl, heteroaryl C₁-C₃alkyl, 3- to 6-membered heterocyclyl, and 3- to 6-membered heterocyclyl C₁-C₃ alkyl.

In an embodiment, unless indicated to the contrary in the definitions of the substituents described above, heteroaryl and aryl groups are independently unsubstituted or substituted with one or more substituents independently selected from the group consisting of hydroxyl, C₁-C₅ alkyl, C₂-C₅ alkenyl, C₂-C₅ alkynyl, C₃-C₅ cycloalkyl, C₁-C₅ alkoxy, halo, —CF₃, —CN, —NH₂, and —NO₂.

In another embodiment, unless indicated to the contrary in the definitions, alkyl, alkenyl, and alkynyl groups are independently unsubstituted or substituted with one or more substituents independently selected from the group consisting of fluoro, C₃-C₅ cycloalkyl, hydroxyl, and C₁-C₅ alkoxy.

In another embodiment, unless indicated to the contrary in the definitions, cycloalkyl and 3-6 membered heterocyclyl are independently unsubstituted or substituted with one or more substituents independently selected from the group consisting of fluoro, C₁-C₅ alkyl, hydroxyl, or C₁-C₅ alkoxy.

In an embodiment, R₁ is C₁-C₈ alkyl, C₂-C₈ alkenyl, C₂-C₈ alkynyl, C₃-C₈ cycloalkyl, C₃-C₆ cycloalkyl C₁-C₅ alkyl, —OR₇, —SR₇, halo, —CF₃, —SF₅, —OCF₃, —CN, —NO₂, or —NR₈R₉. In another embodiment, R₁ is C₁-C₈ alkyl, C₂-C₈ alkenyl, C₂-C₈ alkynyl, C₃-C₈ cycloalkyl, —OR₇, —SR₇, halo, —CF₃, —OCF₃, —CN, —NO₂, or —NR₈R₉. In another embodiment, R₁ is C₁-C₈ alkyl, —S(C₁-C₈ alkyl), halo, —CF₃, —SF₅, —OCF₃, —CN, —NO₂, —NH₂, —NH(C₁-C₄ alkyl), —N(C₁-C₄ alkyl)₂. In another embodiment, R₁ is C₁-C₈ alkyl, —S(C₁-C₈ alkyl), halo, —CF₃, —OCF₃, —CN, —NO₂, —NH₂, —NH(C₁-C₄ alkyl), —N(C₁-C₄ alkyl)₂. In another embodiment, R₁ is C₁-C₈ alkyl, —S(C₁-C₈ alkyl), halo, —CF₃, or —SF₅. In another embodiment, R₁ is C₁-C₈ alkyl, —S(C₁-C₈ alkyl), halo, or —CF₃. In a further embodiment, R₁ is C₁-C₆ alkyl, —S(C₁-C₆ alkyl), halo, or —CF₃. In a further embodiment, R₁ is C₁-C₆ alkyl, halo, or —CF₃. In a still further embodiment, R₁ is C₁-C₈ alkyl or halo. In a further embodiment, R₁ is C₁-C₆ alkyl or halo. In a further embodiment, R₁ is methyl, ethyl, n-propyl, I, Br, or Cl. In a further embodiment, halo is T, Br, or Cl. In an embodiment, halo is Br or Cl, and in a further embodiment, halo is Br.

In an embodiment, R₂ is hydrogen, hydroxyl, C₁-C₃ alkyl, halo, —CF₃, —SF₅, —OCF₃, —CN, —NO₂, —NH₂, —NH(C₁-C₃ alkyl), —N(C₁-C₃ alkyl)₂, C₁-C₃ alkoxy, or —S(C₁-C₃ alkyl). In another embodiment, R₂ is hydrogen, hydroxyl, C₁-C₃ alkyl, halo, —CF₃, —OCF₃, —CN, —NO₂, —NH₂, —NH(C₁-C₃ alkyl), —N(C₁-C₃ alkyl)₂, C₁-C₃ alkoxy, or —S(C₁-C₃ alkyl). In another embodiment, R₂ is hydrogen, hydroxyl, C₁-C₃ alkyl, halo, —CF₃, —CN, —NO₂. —NH₂, C₁-C₃ alkoxy, or —S(C₁-C₃ alkyl). In another embodiment, R₂ is hydrogen, hydroxyl, C₁-C₃ alkyl, halo, —CF₃, or —C₁-C₃ alkoxy. In another embodiment, R₂ is hydrogen or —C₁-C₃ alkoxy. In another embodiment, R₂ is hydrogen or methoxy. In another embodiment, R₂ is hydrogen.

In an embodiment, R₃ is hydrogen, hydroxyl, —OR₁₃, —SR₁₃, —C(O)NH₂, or —O—C(O)R₁₆. In another embodiment, R₃ is hydrogen, hydroxyl, —OR₁₃, or —SR₁₃. In another embodiment, R₃ is hydroxyl, —OR₁₃, or —SR₁₃. In another embodiment, R₃ is hydrogen or OR₁₃. In a further embodiment, R₃ is OR₁₃. In a still further embodiment, R₃ is C₁-C₈ alkoxy or hydrogen, while in another embodiment, R₃ is C₁-C₅ alkoxy or hydrogen. In another embodiment, R₃ is C₁-C₅ alkoxy. In an embodiment, R₃ is C₁-C₃ alkoxy or hydrogen, while in another embodiment, R₃ is C₁-C₃ alkoxy. In a further embodiment, R₃ is hydrogen, methoxy, or ethoxy and in another embodiment, R₃ is ethoxy or methoxy. In a further embodiment, R₃ is hydrogen or methoxy, while in a still further embodiment, R₃ is methoxy.

In an embodiment, R₄ is hydroxyl, —OR₁₉, —SR₁₉, —C(O)NH₂, or —O—C(O)R₂₂. In another embodiment, R₄ is hydroxyl, —OR₁₉, or —SR₁₉. In another embodiment, R₄ is hydroxyl or —OR₁₉. In a further embodiment, R₄ is —OR₁₉. In another embodiment, R₄ is C₁-C₈ alkoxy, while in another embodiment, it is C₁-C₅ alkoxy, and in a further embodiment, R₄ is C₁-C₃ alkoxy. In a still further embodiment, R₄ is methoxy or ethoxy, while in a still further embodiment, R₄ is methoxy.

In an embodiment, R₅ is hydrogen or C₁-C₃ alkyl. In a further embodiment, R₅ is hydrogen or C₁-C₂ alkyl, and in a still further embodiment, R₅ is hydrogen, methyl, or ethyl.

In an embodiment, R₆ is hydrogen or benzyl, wherein the phenyl ring of benzyl is optionally substituted with 1-5 instances of R₆ₐ and each R₆ₐ is independently selected for each occurrence from the group consisting of hydroxyl, C₁-C₃ alkyl, C₂-C₃ alkenyl, C₂-C₃ alkynyl, C₃-C₅ cycloalkyl, halo, —CF₃, —SF₅, —OCF₃, C₁-C₃ alkoxy, —S(C₁-C₃ alkyl), —CN, —NO₂, —NH₂, —NH(C₁-C₃ alkyl), —N(C₁-C₃ alkyl)₂, —C(O)(C₁-C₃ alkyl), —C(O)O(C₁-C₃ alkyl), —O—C(O)(C₁-C₃ alkyl), —C(O)NH₂, —C(O)NH(C₁-C₃ alkyl), and —C(O)N(C₁-C₃ alkyl)₂, or wherein any two adjacent R₆ₐ can be taken together with the atoms on which they are attached to form an optionally substituted 4-6-membered cycloalkyl or 4-6-membered heterocyclyl, wherein said heterocyclyl contains 1 or 2 ring heteroatoms selected from oxygen, nitrogen, and sulfur, and wherein cyclyl and heterocyclyl are as defined hereinabove. It is understood by one of ordinary skill in the art that when two adjacent R₆ₐ substituents taken together form a 4-6-membered cyclyl ring, the ring that is formed is a cycloalkyl ring, but contains a single carbon-carbon double bond between the carbons through which it is fused to the phenyl ring of benzyl. Similarly, when two adjacent R₆ₐ substituents taken together form a 4-6-membered heterocyclyl ring, the ring that is formed is a heterocyclic ring and contains a carbon-carbon double bond between the carbons through which it is fused to the phenyl ring of benzyl. In a further embodiment, R₆ is hydrogen or benzyl, wherein the phenyl ring of benzyl is optionally substituted with 1-5 instances of R₆ₐ and each R₆ₐ is independently selected for each occurrence from the group consisting of hydroxyl, C₁-C₃ alkyl, halo, and C₁-C₃ alkoxy, or wherein any two adjacent R₆ₐ can be taken together with the atoms on which they are attached to form an optionally substituted 4-5-membered cycloalkyl or 4-5-membered heterocyclyl. In an even further embodiment, R₆ is hydrogen or benzyl, wherein the phenyl ring of benzyl is optionally substituted with 1-5 instances of R₆ₐ and each R₆ₐ is independently selected for each occurrence from the group consisting of hydroxyl, halo, methoxy, and ethoxy, or wherein any two adjacent R₆ₐ can be taken together with the atoms on which they are attached to form an optionally substituted 5-membered cycloalkyl or 5-membered heterocyclyl. In another embodiment, R₆ is hydrogen or benzyl, wherein the phenyl ring of benzyl is optionally substituted with 1-5 instances of R₆ₐ and each R₆ₐ is independently selected for each occurrence from the group consisting of hydroxyl, halo, and methoxy, or wherein any two adjacent R₆ₐ can be taken together with the atoms on which they are attached to form an optionally substituted 5-membered cycloalkyl or 5-membered heterocyclyl, fused to the pheny ring. In another embodiment, R₆ is hydrogen or benzyl, wherein the phenyl ring of benzyl is optionally substituted with 1-2 instances of R₆ₐ and each R₆ₐ is independently selected for each occurrence from the group consisting of hydroxyl, fluoro, and methoxy, or any two adjacent R₆ₐ can be taken together with the atoms on which they are attached to form a methylenedioxy ring. In an even further embodiment, R₆ is benzyl, wherein the phenyl ring of benzyl is optionally substituted with 1-2 instances of R₆ₐ and each R₆ₐ is independently selected for each occurrence from the group consisting of hydroxyl, fluoro, and methoxy, or wherein any two adjacent R₆ₐ can be taken together with the atoms on which they are attached to form a methylenedioxy ring. In a further embodiment, R₆ is hydrogen.

In an embodiment, R₃ is —OR₁₃ and R₄ is —OR₁₉, wherein R₁₃ and R₁₉ are as defined hereinabove, and —OR₁₃ and —OR₁₉ may be the same or different. In an embodiment, R₃ and R₄ are independently C₁-C₈ alkoxy, and in another embodiment, are independently C₁-C₅ alkoxy. In an embodiment, R₃ and R₄ are independently methoxy, ethoxy, n-propoxy or isopropoxy. In a further embodiment, R₃ and R₄ are independently methoxy or ethoxy. In a still further embodiment, R₃ and R₄ are the same. In an embodiment, R₃ and R₄ are both C₁-C₈ alkoxy and are both the same, and in another embodiment, R₃ and R₄ are both C₁-C₅ alkoxy and are both the same. In an embodiment, R₃ and R₄ are methoxy, ethoxy, n-propoxy, or isopropoxy and are both the same. In a further embodiment, R₃ and R₄ are methoxy or ethoxy and are both the same. In an embodiment, R₃ and R₄ are both methoxy.

In an embodiment, R₃ is —OR₁₃ and R₄ is —OR₁₉, wherein R₁₃ and R₁₉ are as defined hereinabove, and —OR₁₃ and —OR₁₉ may be the same or different, and R₁ is C₁-C₈ alkyl, C₂-C₈ alkenyl, C₂-C₈ alkynyl, C₃-C₁₀ cycloalkyl, C₃-C₁₀ cycloalkyl C₁-C₈alkyl, —OR₇, —SR₇, halo, —CF₃, —SF₅, —OCF₃, —CN, —NO₂, or —NR₈R₉. In another embodiment, R₁ is C₁-C₈ alkyl, —S(C₁-C₈ alkyl), halo, —CF₃, —SF₅, —OCF₃, —CN, —NO₂, —NH₂, —NH(C₁-C₄ alkyl), —N(C₁-C₄ alkyl)₂ and R₃ is —OR₁₃ and R₄ is —OR₁₉, wherein R₁₃ and R₁₉ are as defined hereinabove, and —OR₁₃ and —OR₁₉ may be the same or different. In another embodiment, R₁ is C₁-C₈ alkyl, —S(C₁-C₈ alkyl), halo, —CF₃, —SF₅, while R₃ is —OR₁₃ and R₄ is —OR₁₉, wherein R₁₃ and R₁₉ are as defined hereinabove, and —OR₁₃ and —OR₁₉ may be the same or different. In a further embodiment, R₁ is C₁-C₆ alkyl, —S(C₁-C₆ alkyl), halo, or —CF₃, and R₃ is —OR₁₃ and R₄ is —OR₁₉, wherein R₁₃ and R₁₉ are as defined hereinabove, and —OR₁₃ and —OR₁₉ may be the same or different. In a still further embodiment, R₁ is C₁-C₈ alkyl or halo, and R₃ is —OR₁₃ and R₄ is —OR₁₉, wherein R₁₃ and R₁₉ are as defined hereinabove, and —OR₁₃ and —OR₁₉ may be the same or different. In a further embodiment, R₁ is C₁-C₆ alkyl or halo and R₃ is —OR₁₃ and R₄ is —OR₁₉, wherein R₁₃ and R₁₉ are as defined hereinabove, and —OR₁₃ and —OR₁₉ may be the same or different. In an embodiment, halo is Br, Cl, or I, and in a further embodiment, halo is Br or Cl, and in a still further embodiment, halo is Br.

In an embodiment, R₃ is C₁-C₈ alkoxy and R₄ is C₁-C₈ alkoxy, and R₃ and R₄ may be the same or different, and R₁ is C₁-C₈ alkyl, C₂-C₈ alkenyl, C₂-C₈ alkynyl, C₃-C₁₀ cycloalkyl, C₃-C₁₀ cycloalkyl C₁-C₈ alkyl, —OR₇, —SR₇, halo, —CF₃, —SF₅, —OCF₃, —CN, —NO₂, or —NR₈R₉. In another embodiment, R₁ is C₁-C₈ alkyl, —S(C₁-C₈ alkyl), halo, —CF₃, —SF₅, —OCF₃, —CN, —NO₂, —NH₂, —NH(C₁-C₄ alkyl), —N(C₁-C₄ alkyl)₂, R₃ is C₁-C₈ alkoxy and R₄ is C₁-C₈ alkoxy, and R₃ and R₄ may be the same or different. In another embodiment, R₁ is C₁-C₈ alkyl, —S(C₁-C₈ alkyl), halo, —CF₃, —SF₅, while R₃ is C₁-C₈alkoxy and R₄ is C₁-C₈alkoxy, and R₃ and R₄ may be the same or different. In a further embodiment, R₁ is C₁-C₆ alkyl, —S(C₁-C₆ alkyl), halo, or —CF₃, and R₃ is C₁-C₈ alkoxy and R₄ is C₁-C₈alkoxy, and R₃ and R₄ may be the same or different. In a still further embodiment, R₁ is C₁-C₈ alkyl or halo, and R₃ is C₁-C₈ alkoxy and R₄ is C₁-C₈ alkoxy and R₃ and R₄ may be the same or different. In a further embodiment, R₁ is C₁-C₆ alkyl or halo and R₃ is C₁-C₈ alkoxy and R₄ is C₁-C₈ alkoxy and R₃ and R₄ may be the same or different. In an embodiment, halo is Br, Cl, or I, and in a further embodiment, halo is Br or Cl, and in a still further embodiment, halo is Br.

In an embodiment, R₃ is C₁-C₅ alkoxy and R₄ is C₁-C₅ alkoxy, and R₃ and R₄ may be the same or different, and R₁ is C₁-C₈alkyl, C₂-C₈ alkenyl, C₂-C₈ alkynyl, C₃-C₁₀ cycloalkyl, C₃-C₁₀ cycloalkyl C₁-C₈ alkyl, —OR₇, —SR₇, halo, —CF₃, —SF₅, —OCF₃, —CN, —NO₂, or —NR₈R₉. In another embodiment, R₁ is C₁-C₈ alkyl, —S(C₁-C₈ alkyl), halo, —CF₃, —SF₅, —OCF₃, —CN, —NO₂, —NH₂, —NH(C₁-C₄ alkyl), —N(C₁-C₄ alkyl)₂, R₃ is C₁-C₈alkoxy and R₄ is C₁-C₈alkoxy, and R₃ and R₄ may be the same or different. In another embodiment, R₁ is C₁-C₈alkyl, —S(C₁-C₈alkyl), halo, —CF₃, —SF₅, while R₃ is C₁-C₅ alkoxy and R₄ is C₁-C₅ alkoxy, and R₃ and R₄ may be the same or different. In a further embodiment, R₁ is C₁-C₆ alkyl, —S(C₁-C₆ alkyl), halo, or —CF₃, and R₃ is C₁-C₅ alkoxy and R₄ is C₁-C₅ alkoxy, and R₃ and R₄ may be the same or different. In a still further embodiment, R₁ is C₁-C₈ alkyl or halo, and R₃ is C₁-C₅ alkoxy and R₄ is C₁-C₅ alkoxy and R₃ and R₄ may be the same or different. In a further embodiment, R₁ is C₁-C₆ alkyl or halo and R₃ is C₁-C₅ alkoxy and R₄ is C₁-C₅ alkoxy and R₃ and R₄ may be the same or different. In an embodiment, halo is Br, Cl, or I, and in a further embodiment, halo is Br or Cl, and in a still further embodiment, halo is Br.

In an embodiment, R₃ is C₁-C₃ alkoxy and R₄ is C₁-C₃ alkoxy, and R₃ and R₄ may be the same or different, and R₁ is C₁-C₈alkyl, C₂-C₈ alkenyl, C₂-C₈ alkynyl, C₃-C₁₀ cycloalkyl, C₃-C₁₀ cycloalkyl C₁-C₈ alkyl, —OR₇, —SR₇, halo, —CF₃, —SF₅, —OCF₃, —CN, —NO₂, or —NR₈R₉. In another embodiment, R₁ is C₁-C₈alkyl, —S(C₁-C₈ alkyl), halo, —CF₃, —SF₅, —OCF₃, —CN, —NO₂, —NH₂, —NH(C₁-C₄ alkyl), —N(C₁-C₄ alkyl)₂, R₃ is C₁-C₅ alkoxy and R₄ is C₁-C₅ alkoxy, and R₃ and R₄ may be the same or different. In another embodiment, R₁ is C₁-C₈ alkyl, —S(C₁-C₈ alkyl), halo, —CF₃, —SF₅, while R₃ is C₁-C₃ alkoxy and R₄ is C₁-C₃ alkoxy, and R₃ and R₄ may be the same or different. In a further embodiment, R₁ is C₁-C₆ alkyl, —S(C₁-C₆ alkyl), halo, or —CF₃, and R₃ is C₁-C₃ alkoxy and R₄ is C₁-C₃ alkoxy, and R₃ and R₄ may be the same or different. In a still further embodiment, R₁ is C₁-C₈ alkyl or halo, and R₃ is C₁-C₃ alkoxy and R₄ is C₁-C₃ alkoxy and R₃ and R₄ may be the same or different. In a further embodiment, R₁ is C₁-C₆ alkyl or halo and R₃ is C₁-C₃ alkoxy and R₄ is C₁-C₃ alkoxy and R₃ and R₄ may be the same or different. In an embodiment, halo is Br, Cl, or I, and in a further embodiment, halo is Br or Cl, and in a still further embodiment, halo is Br.

In an embodiment, R₃ is methoxy and R₄ is methoxy and R₁ is C₁-C₈ alkyl, C₂-C₈ alkenyl, C₂-C₈ alkynyl, C₃-C₁₀ cycloalkyl, C₃-C₁₀ cycloalkyl C₁-C₈alkyl, —OR₇, —SR₇, halo, —CF₃, —SF₅, —OCF₃, —CN, —NO₂, or —NR₈R₉. In another embodiment, R₁ is C₁-C₈ alkyl, —S(C₁-C₈ alkyl), halo, —CF₃, —SF₅, —OCF₃, —CN, —NO₂, —NH₂, —NH(C₁-C₄ alkyl), —N(C₁-C₄ alkyl)₂, R₃ is methoxy and R₄ is methoxy. In another embodiment, R₁ is C₁-C₈ alkyl, —S(C₁-C₈ alkyl), halo, —CF₃, —SF₅, while R₃ is methoxy and R₄ is methoxy. In a further embodiment, R₁ is C₁-C₆ alkyl, —S(C₁-C₆ alkyl), halo, or —CF₃, and R₃ is methoxy and R₄ is methoxy. In a still further embodiment, R₁ is C₁-C₈ alkyl or halo, R₃ is methoxy and R₄ is methoxy. In a further embodiment, R₁ is C₁-C₆ alkyl or halo and R₃ is methoxy and R₄ is methoxy. In an embodiment, halo is Br, Cl, or I, and in a further embodiment, halo is Br or Cl, and in a still further embodiment, halo is Br.

In an embodiment, R₃ is OR₁₃ and R₄ is —OR₁₉, wherein R₁₃ and R₁₉ are as defined hereinabove, and —OR₁₃ and —OR₁₉ may be the same or different, and R₅ is C₁-C₃ alkyl or hydrogen. In an embodiment, R₃ and R₄ are independently C₁-C₈alkoxy and R₅ is C₁-C₃ alkyl or hydrogen. In an embodiment, R₃ and R₄ are independently C₁-C₅ alkoxy and R₅ is C₁-C₃ alkyl or hydrogen. In an embodiment, R₃ and R₄ are independently methoxy, ethoxy, n-propoxy, or isopropoxy and R₅ is C₁-C₃ alkyl or hydrogen. In a further embodiment, R₃ and R₄ are independently methoxy or ethoxy and R₅ is C₁-C₃ alkyl or hydrogen. In a still further embodiment, R₃ and R₄ are the same and R₅ is C₁-C₃ alkyl or hydrogen. In an embodiment, R₃ and R₄ are both C₁-C₈ alkoxy and are both the same and R₅ is C₁-C₃ alkyl or hydrogen. In an embodiment, R₃ and R₄ are both C₁-C₅ alkoxy and are both the same and R₅ is C₁-C₃ alkyl or hydrogen. In an embodiment, R₃ and R₄ are methoxy, ethoxy, n-propoxy, or isopropoxy and are both the same and R₅ is C₁-C₃ alkyl or hydrogen. In a further embodiment, R₃ and R₄ are methoxy or ethoxy and are both the same and R₅ is C₁-C₃ alkyl or hydrogen. In an embodiment, R₃ and R₄ are both methoxy and R₅ is C₁-C₃ alkyl or hydrogen.

In an embodiment, R₃ is —OR₁₃ and R₄ is —OR₁₉, wherein R₁₃ and R₁₉ are as defined hereinabove, and —OR₁₃ and —OR₁₉ may be the same or different, and R₁ is C₁-C₅ alkyl, C₂-C₅ alkenyl, C₂-C₈ alkynyl, C₃-C₁₀cycloalkyl, C₃-C₁₀ cycloalkyl C₁-C₈ alkyl, —OR₇, —SR₇, halo, —CF₃, —SF₅, —OCF₃, —CN, —NO₂, or —NR₈R₉ and R₅ is C₁-C₃ alkyl or hydrogen. In another embodiment, R₁ is C₁-C₈ alkyl, —S(C₁-C₈ alkyl), halo, —CF₃, —SF₅, —OCF₃, —CN, —NO₂, —NH₂, —NH(C₁-C₄ alkyl), —N(C₁-C₄ alkyl)₂ and R₃ is —OR₁₃ and R₄ is —OR₁₉, wherein R₁₃ and R₁₉ are as defined hereinabove, and —OR₁₃ and —OR₁₉ may be the same or different, and R₅ is C₁-C₃ alkyl or hydrogen. In another embodiment, R₁ is C₁-C₈ alkyl, —S(C₁-C₈ alkyl), halo, —CF₃, —SF₅, while R₃ is —OR₁₃ and R₄ is —OR₁₉, wherein R₁₃ and R₁₉ are as defined hereinabove, and —OR₁₃ and —OR₁₉ may be the same or different, and R₅ is C₁-C₃ alkyl or hydrogen. In a further embodiment, R₁ is C₁-C₆ alkyl, —S(C₁-C₆ alkyl), halo, or —CF₃, and R₃ is —OR₁₃ and R₄ is —OR₁₉, wherein R₁₃ and R₁₉ are as defined hereinabove, and —OR₁₃ and —OR₁₉ may be the same or different, and R₅ is C₁-C₃ alkyl or hydrogen. In a still further embodiment, R₁ is C₁-C₈ alkyl or halo, and R₃ is —OR₁₃ and R₄ is —OR₁₉, wherein R₁₃ and R₁₉ are as defined hereinabove, and —OR₁₃ and —OR₁₉ may be the same or different, and R₅ is C₁-C₃ alkyl or hydrogen. In a further embodiment, R₁ is C₁-C₆ alkyl or halo and R₃ is —OR₁₃ and R₄ is —OR₁₉, wherein R₁₃ and R₁₉ are as defined hereinabove, and —OR₁₃ and —OR₁₉ may be the same or different, and R₅ is C₁-C₃ alkyl or hydrogen. In an embodiment, halo is Br, Cl, or I, and in a further embodiment, halo is Br or Cl, and in a still further embodiment, halo is Br.

In an embodiment, R₃ is C₁-C₈ alkoxy and R₄ is C₁-C₈ alkoxy, and R₃ and R₄ may be the same or different, and R₁ is C₁-C₈ alkyl, C₂-C₈ alkenyl, C₂-C₈ alkynyl, C₃-C₁₀ cycloalkyl, C₃-C₁₀ cycloalkyl C₁-C₈alkyl, —OR₇, —SR₇, halo, —CF₃, —SF₅, —OCF₃, —CN, —NO₂, or —NR₈R₉, and R₅ is C₁-C₃ alkyl or hydrogen. In another embodiment, R₁ is C₁-C₈alkyl, —S(C₁-C₈alkyl), halo, —CF₃, —SF₅, —OCF₃, —CN, —NO₂, —NH₂, —NH(C₁-C₄ alkyl), —N(C₁-C₄ alkyl)₂, R₃ is C₁-C₈alkoxy and R₄ is C₁-C₈ alkoxy, and R₃ and R₄ may be the same or different, and R₅ is C₁-C₃ alkyl or hydrogen. In another embodiment, R₁ is C₁-C₈ alkyl, —S(C₁-C₈ alkyl), halo, —CF₃, —SF₅, while R₃ is C₁-C₈ alkoxy and R₄ is C₁-C₈ alkoxy, and R₃ and R₄ may be the same or different, and R₅ is C₁-C₃ alkyl or hydrogen. In a further embodiment, R₁ is C₁-C₆ alkyl, —S(C₁-C₆ alkyl), halo, or —CF₃, and R₃ is C₁-C₈ alkoxy and R₄ is C₁-C₈ alkoxy, and R₃ and R₄ may be the same or different, and R₅ is C₁-C₃ alkyl or hydrogen. In a still further embodiment, R₁ is C₁-C₈ alkyl or halo, and R₃ is C₁-C₈ alkoxy and R₄ is C₁-C₈ alkoxy and R₃ and R₄ may be the same or different, and R₅ is C₁-C₃ alkyl or hydrogen. In a further embodiment, R₁ is C₁-C₆ alkyl or halo and R₃ is C₁-C₈ alkoxy and R₄ is C₁-C₈alkoxy and R₃ and R₄ may be the same or different, and R₅ is C₁-C₃ alkyl or hydrogen. In an embodiment, halo is Br, Cl, or I, and in a further embodiment, halo is Br or Cl, and in a still further embodiment, halo is Br.

In an embodiment, R₃ is C₁-C₅ alkoxy and R₄ is C₁-C₅ alkoxy, and R₃ and R₄ may be the same or different, and R₁ is C₁-C₅ alkyl, C₂-C₅ alkenyl, C₂-C₅ alkynyl, C₃-C₁₀ cycloalkyl, C₃-C₁₀ cycloalkyl C₁-C₈ alkyl, —OR₇, —SR₇, halo, —CF₃, —SF₅, —OCF₃, —CN, —NO₂, or —NR₈R₉, and R₅ is C₁-C₃ alkyl or hydrogen. In another embodiment, R₁ is C₁-C₈alkyl, —S(C₁-C₈alkyl), halo, —CF₃, —SF₅, —OCF₃, —CN, —NO₂, —NH₂, —NH(C₁-C₄ alkyl), —N(C₁-C₄ alkyl)₂, R₃ is C₁-C₅ alkoxy and R₄ is C₁-C₅ alkoxy, and R₃ and R₄ may be the same or different, and R₅ is C₁-C₃ alkyl or hydrogen. In another embodiment, R₁ is C₁-C₈ alkyl, —S(C₁-C₈ alkyl), halo, —CF₃, —SF₅, while R₃ is C₁-C₅ alkoxy and R₄ is C₁-C₅ alkoxy, and R₃ and R₄ may be the same or different, and R₅ is C₁-C₃ alkyl or hydrogen. In a further embodiment, R₁ is C₁-C₆ alkyl, —S(C₁-C₆ alkyl), halo, or —CF₃, and R₃ is C₁-C₅ alkoxy and R₄ is C₁-C₅ alkoxy, and R₃ and R₄ may be the same or different, and R₅ is C₁-C₃ alkyl or hydrogen. In a still further embodiment, R₁ is C₁-C₈alkyl or halo, and R₃ is C₁-C₅ alkoxy and R₄ is C₁-C₅ alkoxy and R₃ and R₄ may be the same or different, and R₅ is C₁-C₃ alkyl or hydrogen. In a further embodiment, R₁ is C₁-C₆ alkyl or halo and R₃ is C₁-C₅ alkoxy and R₄ is C₁-C₅ alkoxy and R₃ and R₄ may be the same or different, and R₅ is C₁-C₃ alkyl or hydrogen. In an embodiment, halo is Br, Cl, or I, and in a further embodiment, halo is Br or Cl, and in a still further embodiment, halo is Br.

In an embodiment, R₃ is C₁-C₃ alkoxy and R₄ is C₁-C₃ alkoxy, and R₃ and R₄ may be the same or different, R₅ is C₁-C₃ alkyl or hydrogen, and R₁ is C₁-C₈ alkyl, C₂-C₈ alkenyl, C₂-C₈ alkynyl, C₃-C₁₀ cycloalkyl, C₃-C₁₀ cycloalkyl C₁-C₈ alkyl, —OR₇, —SR₇, halo, —CF₃, —SF₅, —OCF₃, —CN, —NO₂, or —NR₈R₉. In another embodiment, R₁ is C₁-C₈ alkyl, —S(C₁-C₈ alkyl), halo, —CF₃, —SF₅, —OCF₃, —CN, —NO₂, —NH₂, —NH(C₁-C₄ alkyl), —N(C₁-C₄ alkyl)₂, R₃ is C₁-C₅ alkoxy and R₄ is C₁-C₅ alkoxy, and R₃ and R₄ may be the same or different, and R₅ is C₁-C₃ alkyl or hydrogen. In another embodiment, R₁ is C₁-C₈alkyl, —S(C₁-C₈ alkyl), halo, —CF₃, —SF₅, while R₃ is C₁-C₃ alkoxy and R₄ is C₁-C₃ alkoxy, and R₃ and R₄ may be the same or different, and R₅ is C₁-C₃ alkyl or hydrogen. In a further embodiment, R₁ is C₁-C₆ alkyl, —S(C₁-C₆ alkyl), halo, or —CF₃, and R₃ is C₁-C₃ alkoxy and R₄ is C₁-C₃ alkoxy, and R₃ and R₄ may be the same or different, and R₅ is C₁-C₃alkyl or hydrogen. In a still further embodiment, R₁ is C₁-C₈ alkyl or halo, and R₃ is C₁-C₃ alkoxy and R₄ is C₁-C₃ alkoxy and R₃ and R₄ may be the same or different, and R₅ is C₁-C₃ alkyl or hydrogen. In a further embodiment, R₁ is C₁-C₆ alkyl or halo and R₃ is C₁-C₃ alkoxy and R₄ is C₁-C₃ alkoxy and R₃ and R₄ may be the same or different, and R₅ is C₁-C₃ alkyl or hydrogen. In an embodiment, halo is Br, Cl, or I, and in a further embodiment, halo is Br or Cl, and in a still further embodiment, halo is Br.

In an embodiment, R₃ is methoxy and R₄ is methoxy, R₅ is C₁-C₃ alkyl or hydrogen, and R₁ is C₁-C₈ alkyl, C₂-C₈ alkenyl, C₂-C₈ alkynyl, C₃-C₁₀ cycloalkyl, C₃-C₁₀ cycloalkyl C₁-C₈ alkyl, —OR₇, —SR₇, halo, —CF₃, —SF₅, —OCF₃, —CN, —NO₂, or —NR₈R₉. In another embodiment, R₁ is C₁-C₈ alkyl, —S(C₁-C₈alkyl), halo, —CF₃, —SF₅, —OCF₃, —CN, —NO₂, —NH₂, —NH(C₁-C₄ alkyl), —N(C₁-C₄ alkyl)₂, R₃ is methoxy and R₄ is methoxy, and R₅ is C₁-C₃ alkyl or hydrogen. In another embodiment, R₁ is C₁-C₈ alkyl, —S(C₁-C₈ alkyl), halo, —CF₃, —SF₅, while R₃ is methoxy and R₄ is methoxy, and R₅ is C₁-C₃ alkyl or hydrogen. In a further embodiment, R₁ is C₁-C₆ alkyl, —S(C₁—C₆ alkyl), halo, or —CF₃, and R₃ is methoxy and R₄ is methoxy, and R₅ is C₁-C₃ alkyl or hydrogen. In a still further embodiment, R₁ is C₁-C₈ alkyl or halo, R₃ is methoxy and R₄ is methoxy, and R₅ is C₁-C₃ alkyl or hydrogen. In a further embodiment, R₁ is C₁-C₆ alkyl or halo and R₃ is methoxy and R₄ is methoxy, and R₅ is C₁-C₃ alkyl or hydrogen. In an embodiment, halo is Br, Cl, or I, and in a further embodiment, halo is Br or Cl, and in a still further embodiment, halo is Br.

In an embodiment, R₁ is hydrogen, C₁-C₈alkyl, C₂-C₈ alkenyl, C₂-C₈ alkynyl, C₃-C₁₀ cycloalkyl, —OR₇, —SR₇, halo, —CF₃, —SF₅, —OCF₃, —CN, —NO₂, —NR₈R₉, aryl, heteroaryl, aryl C₁-C₈ alkyl, or heteroaryl C₁-C₈ alkyl;
R₂ is hydrogen, hydroxyl, C₁-C₅ alkyl, halo, —CF₃, —SF₅, —OCF₃, —CN, —NO₂, —NH₂, —NH(C₁-C₈alkyl), —N(C₁-C₈ alkyl)₂, C₁-C₈ alkoxy, or —S(C₁-C₈ alkyl);
R₃ is hydrogen, hydroxyl, —OR₁₃, —SR₁₃, —C(O)NH₂, —O—C(O)R₁₆;
R₄ is hydroxyl, OR₁₉, —SR₁₉, —C(O)NH₂, —O—C(O)R₂₂;
R₅ is hydrogen or C₁-C₂ alkyl; and
R₆ is hydrogen or benzyl, wherein the phenyl ring of benzyl is optionally substituted with 1-5 instances of R₆ₐ and each R₆ₐ is independently selected for each occurrence from the group consisting of hydroxyl, C₁₋₈ alkyl, halo, —CF₃, —SF₅, —OCF₃, C₁-C₈ alkoxy, —S(C₁-C₈ alkyl), —CN, —NO₂, —NH₂, —NH(C₁-C₈ alkyl), —N(C₁-C₈ alkyl)₂, —C(O)(C₁-C₈ alkyl), —C(O)O(C₁-C₈ alkyl), —O—C(O)(C₁-C₈ alkyl), —C(O)NH₂, —C(O)NH(C₁-C₈ alkyl), and —C(O)N(C₁-C₈ alkyl)₂, or wherein any two adjacent R₆ₐ can be taken together with the atoms on which they are attached to form an optionally substituted 4-6-membered cyclyl or 4-6-membered heterocyclyl, wherein said heterocyclyl contains 1 or 2 ring heteroatoms selected from oxygen, nitrogen, and sulfur.

In an embodiment, R₁ is hydrogen, C₁-C₅ alkyl, C₂-C₅ alkenyl, C₂-C₅ alkynyl, C₃-C₁₀ cycloalkyl, 3- to 6-membered heterocyclyl, C₃-C₁₀ cycloalkyl C₁-C₈ alkyl, 3- to 6-membered heterocyclyl C₁-C₈ alkyl, —OR₇, —SR₇, halo, —CF₃, —SF₅, —OCF₃, —CN, —NO₂, —NR₈R₉, aryl, heteroaryl, aryl C₁-C₈ alkyl, or heteroaryl C₁-C₈ alkyl;
R₂ is hydrogen, hydroxyl, C₁-C₃ alkyl, halo, —CF₃, —SF₅, —OCF₃, —CN, —NO₂, —NH₂, —NH(C₁-C₃ alkyl), —N(C₁-C₃ alkyl)₂, C₁-C₃ alkoxy, or —S(C₁-C₃ alkyl);
R₃ is hydrogen, hydroxyl, —OR₁₃, —SR₁₃, —C(O)NH₂, —O—C(O)R₁₆;
R₄ is hydroxyl, OR₁₉, —SR₁₉, —C(O)NH₂, —O—C(O)R₂₂;
R₅ is hydrogen or C₁-C₂ alkyl; and
R₆ is hydrogen or benzyl, wherein the phenyl ring of benzyl is optionally substituted with 1-5 instances of R₆ₐ and each R₆ₐ is independently selected for each occurrence from the group consisting of hydroxyl, C₁₋₃ alkyl, halo, —CF₃, —SF₅, —OCF₃, C₁-C₈alkoxy, —S(C₁-C₃ alkyl), —CN, —NO₂, —NH₂, —NH(C₁-C₃ alkyl), —N(C₁-C₃ alkyl)₂, —C(O)(C₁-C₃ alkyl), —C(O)O(C₁-C₅ alkyl), —O—C(O)(C₁-C₈ alkyl), —C(O)NH₂, —C(O)NH(C₁-C₈ alkyl), and —C(O)N(C₁-C₃ alkyl)₂, or wherein any two adjacent R₆ₐ can be taken together with the atoms on which they are attached to form an optionally substituted 4-6-membered cyclyl or 4-6-membered heterocyclyl, wherein said heterocyclyl contains 1 or 2 ring heteroatoms selected from oxygen, nitrogen, and sulfur.

In a further embodiment, R₁ is C₁-C₈ alkyl, C₂-C₈ alkenyl, C₂-C₈ alkynyl, C₃-C₈ cycloalkyl, C₃-C₆ cycloalkyl C₁-C₈alkyl, —OR₇, —SR₇, halo, —CF₃, —SF₅, —OCF₃, —CN, —NO₂, or —NR₈R₉;
R₂ is hydrogen or C₁-C₃ alkoxy;
R₃ is hydrogen or C₁-C₃ alkoxy;
R₄ is C₁-C₃ alkoxy;
R₅ is hydrogen or C₁-C₂ alkyl;
R₆ is hydrogen or benzyl, wherein the phenyl ring of benzyl is optionally substituted with 1-5 instances of R₆ₐ and each R₆ₐ is independently selected for each occurrence from the group consisting of hydroxyl, C₁-C₃ alkyl, halo, and C₁-C₃ alkoxy, or wherein any two adjacent R₆ₐ can be taken together with the atoms on which they are attached to form an optionally substituted 4-5-membered cyclyl or 4-5-membered heterocyclyl; and
   R₇, R₈, and R₉ are independently selected from the group consisting of hydrogen, C₁-C₈alkyl, C₂-C₈ alkenyl, C₂-C₈ alkynyl, C₃-C₈ cycloalkyl, and C₃-C₆ cycloalkyl C₁-C₅ alkyl.

In a still further embodiment, R₁ is C₁-C₈ alkyl, —S(C₁-C₈ alkyl), halo, —CF₃, —SF₅, —OCF₃, —CN, —NO₂, —NH₂, —NH(C₁-C₄ alkyl), —N(C₁-C₄ alkyl)₂;
R₂ is hydrogen or methoxy;
R₃ is hydrogen or methoxy;
R₄ is methoxy;
R₅ is hydrogen or C₁-C₂ alkyl; and
R₆ is hydrogen or benzyl, wherein the phenyl ring of benzyl is optionally substituted with 1-5 instances of R₆ₐ and each R₆ₐ is independently selected for each occurrence from the group consisting of hydroxyl, halo, and methoxy, or wherein any two adjacent R₆ₐ can be taken together with the atoms on which they are attached to form an optionally substituted 5-membered cyclyl or 5-membered heterocyclyl.

In another embodiment, R₁ is C₁-C₈alkyl, —S(C₁-C₈ alkyl), halo, —CF₃, —SF₅,
R₂ is hydrogen or methoxy;
R₃ is hydrogen or methoxy;
R₄ is methoxy;
R₅ is hydrogen or C₁-C₂ alkyl; and
R₆ is hydrogen or benzyl, wherein the phenyl ring of benzyl is optionally substituted with 1-2 instances of R₆ₐ and each R₆ₐ is independently selected for each occurrence from the group consisting of hydroxyl, fluoro, and methoxy, or any two adjacent R₆ₐ can be taken together with the atoms on which they are attached to form a methylenedioxy ring.

In a further embodiment, R₁ is C₁-C₈ alkyl, —S(C₁-C₈ alkyl), halo, or —CF₃ and R₂, R₃, R₄, R₅ and R₆ are as defined herein. In a further embodiment, R₁ is C₁-C₆ alkyl, —S(C₁-C₆ alkyl), halo, or —CF₃ and R₂, R₃, R₄, R₅ and R₆ are as defined herein. In a further embodiment, R₂ is hydrogen, and R₁, R₃, R₄, R₅ and R₆ are as defined herein. In a still another embodiment, R₃ and R₄ are methoxy, and R₁, R₂, R₅ and R₆ are as defined herein. In an even further embodiment, R₅ is hydrogen, methyl, or ethyl, and R₁, R₂, R₃, R₄ and R₆ are as defined herein. In a still further embodiment, R₆ is hydrogen, and R₁, R₂, R₃, R₄ and R₅ are as defined herein. In another embodiment, R₆ is benzyl, wherein the phenyl ring of benzyl is optionally substituted with 1-2 instances of R₆ₐ and each R₆ₐ is independently selected for each occurrence from the group consisting of hydroxyl, fluoro, and methoxy, or wherein any two adjacent R₆ₐ can be taken together with the atoms on which they are attached to form a methylenedioxy ring, and R₁, R₂, R₃, R₄ and R₅ are as defined herein.

In an embodiment, R₃ is —OR₁₃, wherein R₁₃ is C₁-C₅ alkyl, e.g., C₁-C₅ alkoxy, e.g., methoxy; R₄ is —OR₁₉, wherein R₁₉ is C₁-C₅ alkyl, e.g. C₁-C₅ alkoxy, e.g., methoxy, R₂ is hydrogen, R₁ is C₁-C₈ alkyl, halo, such as chloro or bromo, —CF₃, —SR₇, wherein R₇ is C₁-C₈ alkyl, i.e., C₁-C₅ alkylthio; R₅ is hydrogen or C₁-C₃ alkyl, and R₆ is hydrogen or benzyl, wherein the phenyl ring of benzyl is optionally substituted with 1-2 instances of R₆ₐ and each R₆ₐ is independently selected for each occurrence from the group consisting of hydroxyl, halo such as fluoro, and C₁-C₅ alkoxy, such as methoxy.

In a further embodiment, the compounds of Formula I have the structure:

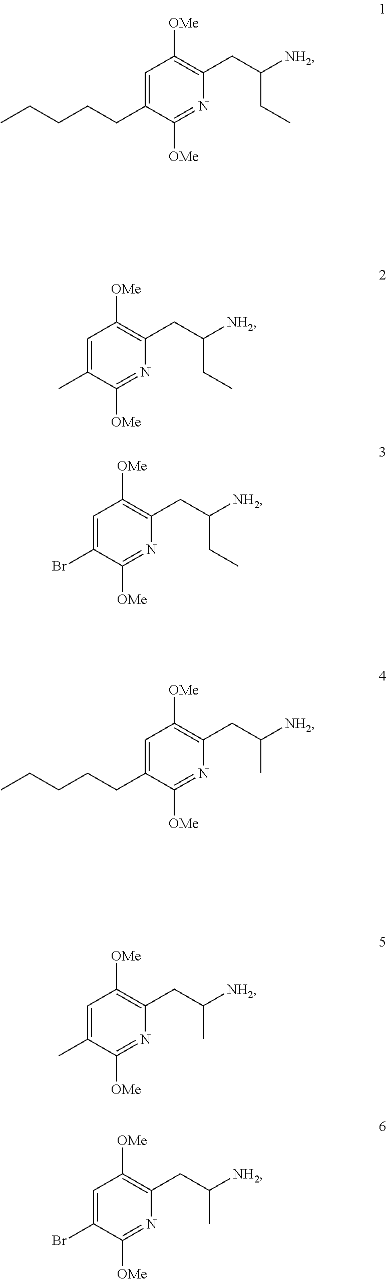

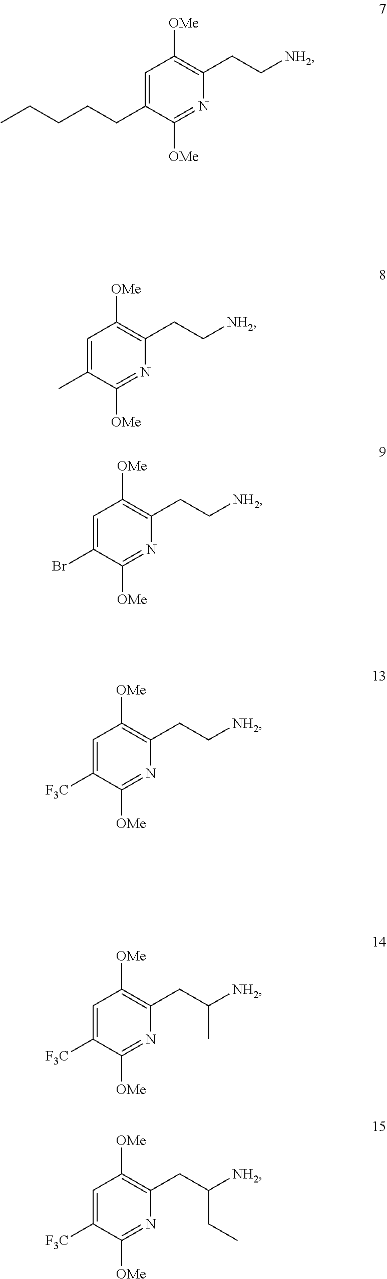

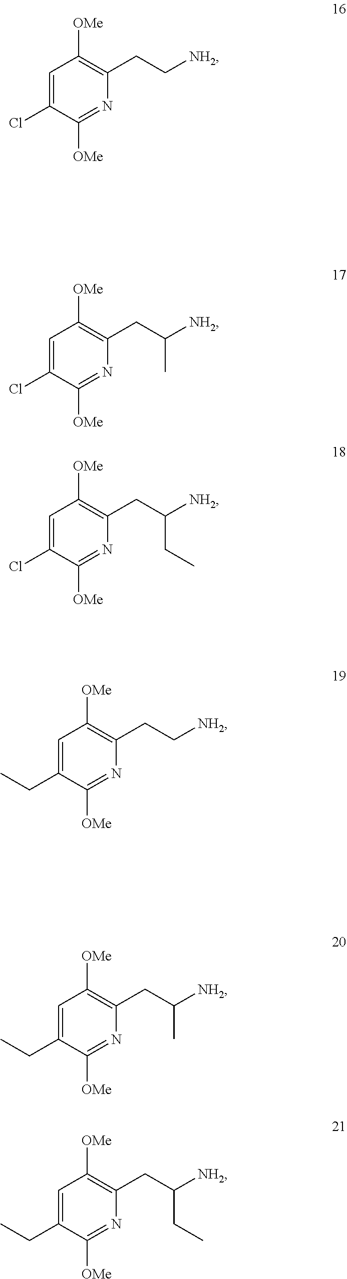

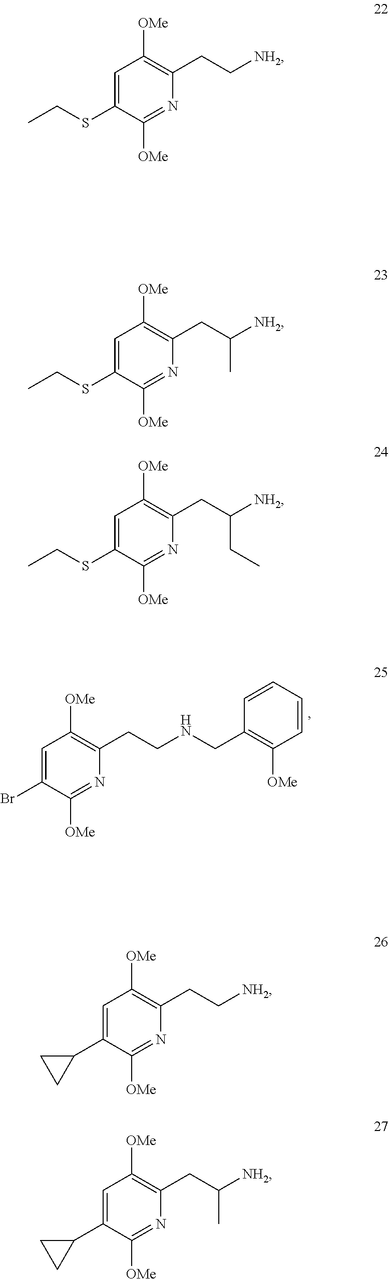

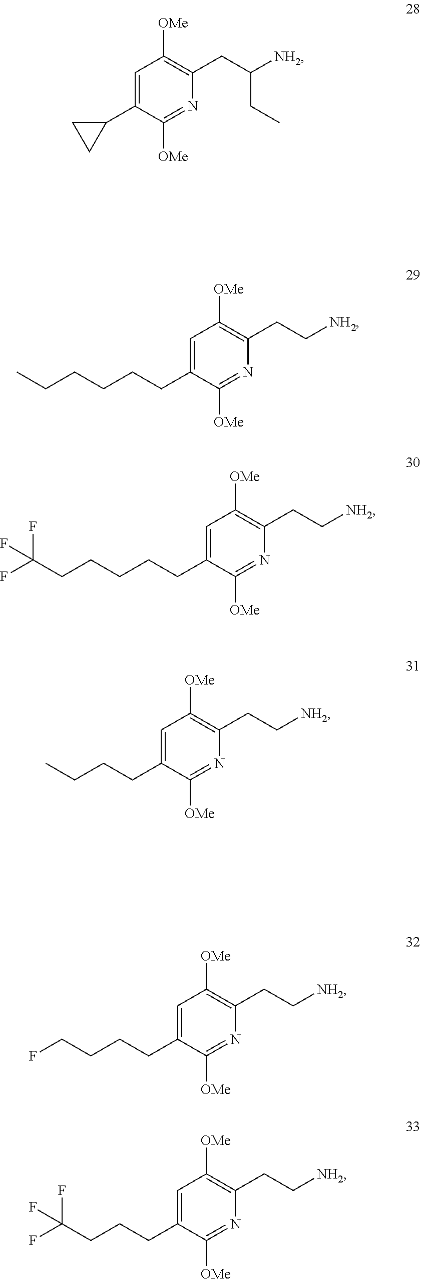 [SMILES: CCC(N)Cc1nc(OC)c(C2CC2)cc1OC ; CCCCCCc1cc(OC)c(CCN)nc1OC ; COc1cc(CCCCCC(F)(F)F)c(OC)nc1CCN ; CCCCc1cc(OC)c(CCN)nc1OC ; COc1cc(CCCCF)c(OC)nc1CCN ; COc1cc(CCCC(F)(F)F)c(OC)nc1CCN]

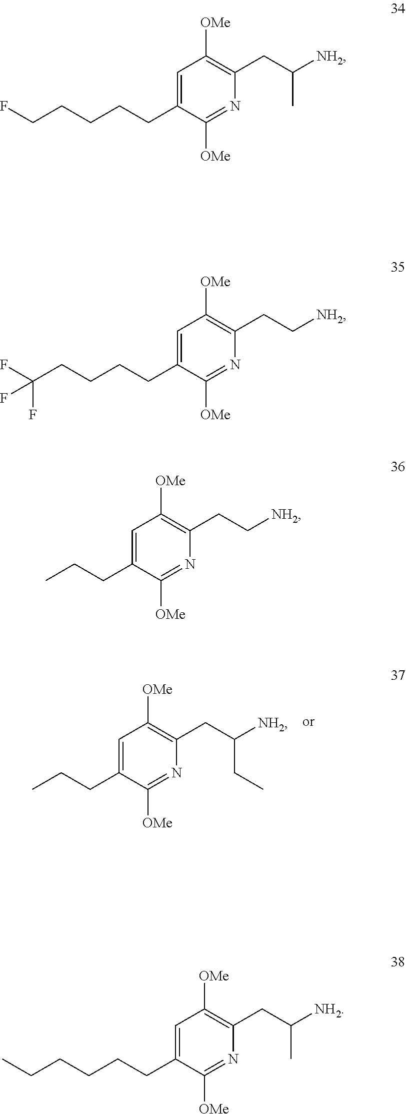

It is to be understood that all combinations and permutations of R₁, R₂, R₃, R₄, R₅, and R₆ are contemplated within the compounds of the present disclosure.

The present disclosure includes isotopes of atoms occurring on compounds of Formula I and the various embodiments described herein. As defined herein, isotopes include those atoms having the same atomic number, but different mass numbers. By way of general example and without limitation, isotopes of hydrogen include protium, deuterium, and tritium, and isotopes of carbon include carbon-12, carbon-13, and carbon-14.

It is to be understood that any notation of a carbon in structures throughout this disclosure, when used without further notation, is intended to represent all isotopes of carbon, such as ¹²C, ¹³C, or ¹⁴C. Furthermore, compounds containing ¹³C or ¹⁴C may specifically have the structure of any of the compounds disclosed herein.

It is also understood that any notation of a hydrogen in structures in this disclosure, when used without further notation, is intended to represent all isotopes of hydrogen, such as ¹H, ²H, or ³H. Furthermore, any compounds containing ²H or ³H may specifically have the structure of any of the compounds disclosed herein.

Isotopically-labeled compounds may generally be prepared by conventional techniques known to the skilled artisan using appropriate isotopically labeled reagents in place of the non-labeled reagents employed.

It is further understood and appreciated that in some embodiments, compounds of the present disclosure may have at least one chiral center and therefore, can exist as enantiomers and/or diastereomers. It is to be understood that all such isomers and mixtures thereof in any proportion are encompassed within the scope of the present disclosure. It is to be further understood that while the stereochemistry of the compounds may be as shown in any given compound listed herein, such compounds may also contain certain amounts (for example, less than 20%, suitably less than 10%, more suitably less than 5%) of compounds of the present disclosure having an alternate stereochemistry. It is intended that any optical isomers, as separated, pure, or partially purified optical isomers or racemic or scalemic mixtures thereof are included within the scope of the present disclosure.

The compounds of the present disclosure are suitably formulated in a conventional manner into compositions using one or more carriers. Accordingly, the present disclosure also includes a composition comprising one or more compounds of the disclosure and a pharmaceutically acceptable carrier. The compounds of the present disclosure are suitably formulated into pharmaceutical compositions for administration to subjects in a biologically compatible form suitable for administration in vivo. Accordingly, the present disclosure further includes a pharmaceutical composition comprising one or more compounds of the disclosure and a pharmaceutically acceptable carrier. In embodiments of the present disclosure, the pharmaceutical compositions are used in the treatment of any of the diseases, disorders, or conditions described herein.

The compounds of the disclosure are administered to a subject in a variety of forms depending on the selected route of administration, as will be understood by those skilled in the art. For example, a compound of the present disclosure is administered by oral, inhalation, intravenous, vaporization, parenteral, buccal, sublingual, insufflation, epidurally, nasal, rectal, vaginal, patch, pump, minipump, topical or transdermal administration and the pharmaceutical compositions formulated accordingly. In some embodiments, administration is by means of a pump for periodic or continuous delivery. Conventional procedures and ingredients for the selection and preparation of suitable compositions are described, for example, in Remington's Pharmaceutical Sciences (2000-20^{th} edition) and in The United States Pharmacopeia: The National Formulary (USP 24 NF19) published in 1999.

Parenteral administration includes systemic delivery routes other than the gastrointestinal (GI) tract and includes, for example intravenous, intra-arterial, intraperitoneal, subcutaneous, intramuscular, transepithelial, nasal, intrapulmonary (for example, by use of an aerosol), intrathecal, rectal, and topical (including the use of a patch or other transdermal delivery device) modes of administration. Parenteral administration may be by continuous infusion over a selected period of time.

In some embodiments, a compound of the present disclosure is orally administered, for example, with an inert diluent or with an assimilable edible carrier, or it is enclosed in hard- or soft-shell gelatin capsules, or it is compressed into tablets, or it is incorporated directly with the food of the diet. In some embodiments, the compound is incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate, and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose, or calcium phosphate); lubricants (e.g., magnesium stearate, talc, or silica); disintegrants (e.g., potato starch or sodium starch glycolate); wetting agents (e.g., sodium lauryl sulphate); or solvents (e.g., medium chain triglycerides, ethanol, water). In embodiments, the tablets are coated by methods well known in the art. In the case of tablets, capsules, caplets, pellets, or granules for oral administration, pH sensitive enteric coatings, such as Eudragits® designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions are formulated, for example, as liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. In some embodiments, liposomes are formed from a variety of phospholipids, such as cholesterol, stearylamine, or phosphatidylcholines. For oral administration in a capsule form, useful carriers, solvents, or diluents include lactose, medium chain triglycerides, ethanol, and dried corn starch.

In some embodiments, liquid preparations for oral administration take the form of, for example, solutions, syrups, or suspensions, or they are suitably presented as a dry product for constitution with water or other suitable vehicle before use. When aqueous suspensions and/or emulsions are administered orally, the compound of the disclosure is suitably suspended or dissolved in an oily phase that is combined with emulsifying and/or suspending agents. If desired, certain sweetening and/or flavoring and/or coloring agents are added. Such liquid preparations for oral administration are prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., medium chain triglycerides, almond oil, oily esters, or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the present disclosure and use the lyophilizates obtained, for example, for the preparation of products for injection.

In some embodiments, a compound of the present disclosure is administered parenterally. For example, solutions of a compound of the present disclosure are prepared in water suitably mixed with a surfactant such as hydroxypropylcellulose. In some embodiments, dispersions are prepared in glycerol, liquid polyethylene glycols, DMSO and mixtures thereof, with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compounds of the disclosure are usually prepared and the pHs of the solutions are suitably adjusted and buffered. For intravenous use, the total concentration of solutes should be controlled to render the preparation isotonic. For ocular administration, ointments or droppable liquids are delivered, for example, by ocular delivery systems known to the art such as applicators or eye droppers. In some embodiments, such compositions include mucomimetics such as hyaluronic acid, chondroitin sulfate, hydroxypropyl methylcellulose, or polyvinyl alcohol, preservatives such as sorbic acid, EDTA, or benzyl chromium chloride, and the usual quantities of diluents or carriers. For pulmonary administration, diluents or carriers will be selected to be appropriate to allow the formation of an aerosol.

In some embodiments, a compound of the present disclosure is formulated for parenteral administration by injection, including using conventional catheterization techniques or infusion. Formulations for injection are, for example, presented in unit dosage form, e.g., in ampoules or in multi-dose containers, with an added preservative. In some embodiments, the compositions take such forms as sterile suspensions, solutions, or emulsions in oily or aqueous vehicles and contain formulating agents such as suspending, stabilizing, and/or dispersing agents. In all cases, the form must be sterile and must be fluid to the extent that easy syringability exists. Alternatively, the compounds of the disclosure are suitably in a sterile powder form for reconstitution with a suitable vehicle, e.g., sterile pyrogen-free water, before use.

In some embodiments, compositions for nasal administration are conveniently formulated as aerosols, drops, gels and powders. For intranasal administration or administration by inhalation, the compounds of the disclosure are conveniently delivered in the form of a solution, dry powder formulation, or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which, for example, take the form of a cartridge or refill for use with an atomizing device. Alternatively, the sealed container is a unitary dispensing device such as a single dose nasal inhaler or an aerosol dispenser fitted with a metering valve which is intended for disposal after use. Where the dosage form comprises an aerosol dispenser, it will contain a propellant which is, for example, a compressed gas such as compressed air or an organic propellant such as a fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. In some embodiments, the pressurized container or nebulizer contains a solution or suspension of the active compound. Capsules and cartridges (made, for example, from gelatin) for use in an inhaler or insufflator are, for example, formulated containing a powder mix of a compound of the disclosure and a suitable powder base such as lactose or starch. The aerosol dosage forms can also take the form of a pump-atomizer.

Compositions suitable for buccal or sublingual administration include tablets, lozenges, films, and pastilles, wherein a compound of the disclosure is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of the present disclosure are useful for vaginal, urethral, and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16^{th} Ed., Mack Publishing, Easton, Pa., 1980, pp. 1530-1533 for further discussion of suppository dosage forms.

In some embodiments a compound of the present disclosure is coupled with soluble polymers as targetable drug carriers. Such polymers include, for example, polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues, and the like. Furthermore, in some embodiments, a compound of the present disclosure is coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates, and crosslinked or amphipathic block copolymers of hydrogels, and the like.

A compound of the disclosure including pharmaceutically acceptable salts, solvates and/or prodrugs thereof is suitably used on their own but will generally be administered in the form of a pharmaceutical composition in which the one or more compounds of the disclosure (the active ingredient) is in association with a pharmaceutically acceptable carrier. Depending on the mode of administration, the pharmaceutical composition will comprise from about 0.05 wt % to about 99 wt % or from about 0.10 wt % to about 70 wt %, of the active ingredient and from about 1 wt % to about 99.95 wt % or from about 30 wt % to about 99.90 wt % of a pharmaceutically acceptable carrier, all percentages by weight being based on the total composition.

In the above, the term “a compound” also includes embodiments wherein one or more compounds are referenced.

The compounds of Formula I and the pharmaceutically acceptable salt thereof are serotonin receptor binding agents that act as agonists or partial agonists at a serotonin receptor.

Accordingly, the present disclosure includes a method for activating a serotonin receptor in a cell, either in a biological sample or in a subject, comprising administering an effective amount of one or more compounds of Formula I or a pharmaceutically acceptable salt thereof to the cell.

Since the compounds of Formula I or pharmaceutically acceptable salts thereof are capable of activating a serotonin receptor, they are also useful for treating diseases, disorders, or conditions by activating a serotonin receptor. Therefore, the compounds of Formula I are useful as medicaments.

The present disclosure also includes a method of treating a disease, disorder, or condition by activation of a serotonin receptor comprising administering a therapeutically effective amount of one or more compounds of Formula I or a pharmaceutically acceptable salt thereof to a subject in need thereof. In some embodiments, the serotonin receptor is the 5-HT2A receptor. Accordingly, the present disclosure includes a method for activating 5-HT2A receptors in a cell, either in a biological sample or in a patient, comprising administering an effective amount of one or more compounds of Formula I or a pharmaceutically acceptable salt thereof to the cell.

The present disclosure also includes a method of treating a disease, disorder, or condition by activation of 5-HT2A receptors, comprising administering a therapeutically effective amount of one or more compounds of Formula I or a pharmaceutically acceptable salt thereof to a subject in need thereof.

In an embodiment, the present disclosure relates to a method of treating a psychiatric disorder in a subject which comprises administering to said subject in need thereof a therapeutically effective amount of a compound of Formula I or a pharmaceutically acceptable salt thereof. Contemplated psychiatric disorders include Depressive Disorders, e.g., Major Depressive Disorder, Persistent Depressive Disorder, Postpartum Depression, Premenstrual Dysphoric Disorder, Seasonal Affective Disorder, Psychotic Depression, Disruptive Mood Dysregulation Disorder, Substance/Medication-Induced Depressive Disorder, and Depressive Disorder Due to Another Medical Condition. In addition, in another embodiment, the present disclosure relates to a method for treating refractory depression in a subject, e.g., patients suffering from a depressive disorder that does not, and/or has not, responded to adequate courses of at least one, or at least two, other antidepressant compounds or therapeutics comprising administering to said subject in need thereof a therapeutically effective amount of a compound of Formula I or a pharmaceutically acceptable salt thereof. As used herein “depressive disorder” encompasses refractory depression.

In another embodiment, the psychiatric disorder which the compounds of Formula I or pharmaceutically acceptable salt thereof is useful for treating in a subject is Bipolar and Related Disorders, e.g., Bipolar I Disorder, Bipolar II Disorder, Cyclothymic Disorder, Substance/Medication-Induced Bipolar and Related Disorder, and Bipolar and Related Disorder Due to Another Medical Condition.

In another embodiment, the compounds of Formula I or pharmaceutically acceptable salt thereof is used to a treat psychiatric disorder including Substance Abuse-Related Disorders, e.g., preventing a substance abuse craving, diminishing a substance abuse craving, and/or facilitating substance abuse cessation or withdrawal, by administering a therapeutically effective amount of the compounds of Formula I or pharmaceutically acceptable salt to a subject in need thereof. Substance abuse disorders involve abuse of psychoactive compounds such as alcohol, caffeine, cannabis, inhalants, opioids, sedatives, hypnotics, anxiolytics, stimulants, nicotine, and tobacco. As used herein, the term “substance” or “substances,” with respect to the terms “substance abuse” or “substances abuse” or with respect to substance abuse disorders are psychoactive compounds which can be addictive such as alcohol, caffeine, cannabis, hallucinogens, inhalants, opioids, sedatives, hypnotics, anxiolytics, stimulants, nicotine, and tobacco. For example, in an embodiment, the methods and compositions of the present disclosure may be used to facilitate smoking cessation or cessation of opioid use.

In some embodiments, the psychiatric disorder that the compound of Formula I or pharmaceutically acceptable salt thereof is useful for treating by administering a therapeutically effective amount of said compound of Formula I or pharmaceutically acceptable salt thereof to a subject in need thereof includes Anxiety Disorders, e.g., Separation Anxiety Disorder, Selective Mutism, Specific Phobia, Social Anxiety Disorder (Social Phobia), Panic Disorder, Panic Attack, Agoraphobia, Generalized Anxiety Disorder, Substance/Medication-Induced Anxiety Disorder, and Anxiety Disorder Due to Another Medical Condition.

In some embodiments, the psychiatric disorder that the compound of Formula I or pharmaceutically acceptable salt thereof is useful for treating by administering a therapeutically effective amount of said compound of Formula I or pharmaceutically acceptable salt thereof to a subject in need thereof includes Obsessive-Compulsive and Related Disorders, e.g., Obsessive Compulsive Disorder, Body Dysmorphic Disorder, Hoarding Disorder, Trichotillomania (HairPulling Disorder), Excoriation (Skin-Picking) Disorder, Substance/Medication-Induced Obsessive-Compulsive and Related Disorder and Obsessive-Compulsive and Related Disorder Due to Another Medical Condition.

In some embodiments, the psychiatric disorder that the compound of Formula I or pharmaceutically acceptable salt thereof is useful for treating by administering a therapeutically effective amount of said compound of Formula I or pharmaceutically acceptable salt thereof to a subject in need thereof includes Trauma- and Stressor-Related Disorders, e.g., Reactive Attachment Disorder, Disinhibited Social Engagement Disorder, Posttraumatic Stress Disorder, Acute Stress Disorder, and Adjustment Disorders.

In some embodiments, the psychiatric disorder that the compound of Formula I or pharmaceutically acceptable salt thereof is useful for treating by administering a therapeutically effective amount of said compound of Formula I or pharmaceutically acceptable salt thereof to a subject in need thereof includes Feeding and Eating Disorders, e.g., Anorexia Nervosa, Bulimia Nervosa, Binge-Eating Disorder, Pica, Rumination Disorder, and Avoidant/Restrictive Food Intake Disorder.

In some embodiments, the psychiatric disorder that the compound of Formula I or pharmaceutically acceptable salt thereof is useful for treating by administering a therapeutically effective amount of said compound of Formula I or pharmaceutically acceptable salt thereof to a subject in need thereof includes Neurocognitive Disorders, e.g., Delirium, Major Neurocognitive Disorder, Mild Neurocognitive Disorder, Major or Mild Neurocognitive Disorder Due to Alzheimer's Disease, Major or Mild Frontotemporal Neurocognitive Disorder, Major or Mild Neurocognitive Disorder With Levvy Bodies, Major or Mild Vascular Neurocognitive Disorder, Major or Mild Neurocognitive Disorder Due to Traumatic Brain Injury, Substance/Medication-Induced Major or Mild Neurocognitive Disorder, Major or Mild Neurocognitive Disorder Due to HIV Infection, Major or Mild Neurocognitive Disorder Due to Prion Disease, Major or Mild Neurocognitive Disorder Due to Parkinson's Disease, Major or Mild Neurocognitive Disorder Due to Huntington's Disease, Major or Mild Neurocognitive Disorder Due to Another Medical Condition, and Major or Mild Neurocognitive Disorder Due to Multiple Etiologies.

In some embodiments, the psychiatric disorder that the compound of Formula I or pharmaceutically acceptable salt thereof is useful for treating by administering a therapeutically effective amount of said compound of Formula I or pharmaceutically acceptable salt thereof to a subject in need thereof includes Neurodevelopmental Disorders, e.g., Autism Spectrum Disorder, Attention-Deficit/Hyperactivity Disorder, Stereotypic Movement Disorder, Tic Disorders, Tourette's Disorder, Persistent (Chronic) Motor or Vocal Tic Disorder, and Provisional Tic Disorder.

In some embodiments, the psychiatric disorder that the compound of Formula I or pharmaceutically acceptable salt thereof is useful for treating by administering a therapeutically effective amount of the compound of Formula I or pharmaceutically acceptable salt thereof to a subject in need thereof includes Personality Disorders, e.g., Borderline Personality Disorder.

In some embodiments, the psychiatric disorder that the compound of Formula I or pharmaceutically acceptable salt thereof is useful for treating by administering a therapeutically effective amount of said compound of Formula I or pharmaceutically acceptable salt thereof to a subject in need thereof includes Sexual Dysfunctions, e.g., Delayed Ejaculation, Erectile Disorder, Female Orgasmic Disorder, Female Sexual Interest/Arousal Disorder, Genito-Pelvic Pain/Penetration Disorder, Male Hypoactive Sexual Desire Disorder, Premature (Early) Ejaculation, and Substance/Medication-Induced Sexual Dysfunction.

In some embodiments, the psychiatric disorder that the compound of Formula I or pharmaceutically acceptable salt thereof is useful for treating by administering a therapeutically effective amount of said compound of Formula I or pharmaceutically acceptable salt thereof to a subject in need thereof includes Gender Dysphoria, e.g., Gender Dysphoria.

In an embodiment, the compounds of Formula I or pharmaceutically acceptable salts thereof are useful for treating depression, a mood disorder, an anxiety disorder, or a substance use disorder, and any symptom associated therewith in a subject in need thereof, comprising administering a therapeutically effective amount of the compound of Formula I.

In some embodiments, the compounds of the present disclosure or pharmaceutically acceptable salts thereof are useful for preventing, treating, and/or reducing the severity of a mental illness, disorder, and/or condition in a subject. Therefore, in some embodiments, the disease, disorder, or condition that is treated by activation of a serotonin receptor is a mental illness. Accordingly, the present disclosure also includes a method of treating a mental illness comprising administering a therapeutically effective amount of one or more compounds of the disclosure to a subject in need thereof. The present disclosure also includes a use of one or more compounds of the present disclosure or pharmaceutically acceptable salts thereof for treatment of a mental illness, as well as a use of one or more compounds of the disclosure for the preparation of a medicament for treatment of a mental illness. The disclosure further includes one or more compounds of the disclosure for use in treating a mental illness.

In some embodiments, the mental illness is selected from anxiety disorders such as generalized anxiety disorder, panic disorder, social anxiety disorder and specific phobias; depression such as, hopelessness, loss of pleasure, fatigue and suicidal thoughts; mood disorders, such as depression, bipolar disorder, cancer-related depression, anxiety and cyclothymic disorder; psychotic disorders, such as hallucinations, delusions, schizophrenia; impulse control and addiction disorders, such as pyromania (starting fires), kleptomania (stealing) and compulsive gambling; alcohol addiction; drug addiction, such as opioid addiction; personality disorders, such as antisocial personality disorder, obsessive-compulsive personality disorder and paranoid personality disorder; obsessive-compulsive disorder (OCD), such as thoughts or fears that cause a subject to perform certain rituals or routines; post-traumatic stress disorder (PTSD); stress response syndromes (formerly called adjustment disorders); dissociative disorders, formerly called multiple personality disorder, or “split personality,” and depersonalization disorder; factitious disorders; sexual and gender disorders, such as sexual dysfunction, gender identity disorder and the paraphilia's; somatic symptom disorders, formerly known as a psychosomatic disorder or somatoform disorder; and combinations thereof.

In some embodiments, the disease, disorder, or condition that is treated by activation of a serotonin receptor is neurodegeneration. In some embodiments, the disease, disorder, or condition that is treated by activation of a serotonin receptor is reduced brain-derived neurotrophic factor (BDNF), mammalian target of rapamycin (mTOR) activation, and/or inflammation.

In some embodiments, the disease, disorder or condition that is treated by activation of a serotonin receptor comprises cognitive impairment; ischemia including stroke; neurodegeneration; refractory substance abuse disorders; sleep disorders; pain, such as social pain, acute pain, cancer pain, chronic pain, breakthrough pain, bone pain, soft tissue pain, nerve pain, referred pain, phantom pain, neuropathic pain, cluster headaches and migraine; obesity and eating disorders; epilepsies and seizure disorders; neuronal cell death; excitotoxic cell death; or a combination thereof. In some embodiments, the mental illness is selected from hallucinations, delusions, and a combination thereof.

In some embodiments, the hallucinations are selected from visual hallucinations, auditory hallucinations, olfactory hallucinations, gustatory hallucinations, tactile hallucinations, proprioceptive hallucinations, equilibrioceptive hallucinations, nociceptive hallucinations, thermoceptive hallucinations and chronoceptive hallucinations, and a combination thereof.

In some embodiments, the disease, disorder, or condition that is treated by activation of a serotonin receptor is psychosis or psychotic symptoms. Accordingly, the present disclosure also includes a method of treating psychosis or psychotic symptoms comprising administering a therapeutically effective amount of one or more compounds of the disclosure or pharmaceutically acceptable salts thereof to a subject in need thereof.

The present disclosure also includes a use of one or more compounds of the present disclosure or pharmaceutically acceptable salts thereof for treatment of psychosis or psychotic symptoms, as well as a use of one or more compounds of the disclosure for the preparation of a medicament for treatment of psychosis or psychotic symptoms. The disclosure further includes one or more compounds of the disclosure or pharmaceutically acceptable salt thereof for use in treating psychosis or psychotic symptoms.

In some embodiments, administering to said subject in need thereof a therapeutically effective amount of the compounds of the present disclosure or pharmaceutically acceptable salt thereof does not result in a worsening of psychosis or psychotic symptoms such as, but not limited to, hallucinations and delusions. In some embodiments, administering to said subject in need thereof a therapeutically effective amount of the compounds of the disclosure or pharmaceutically acceptable salts thereof results in an improvement of psychosis or psychotic symptoms such as, but not limited to, hallucinations and delusions. In some embodiments, administering to said subject in need thereof a therapeutically effective amount of the compounds of the disclosure results in an improvement of psychosis or psychotic symptoms.

In some embodiments, the compounds of the present disclosure and pharmaceutically acceptable salts thereof are useful for treating a central nervous system (CNS) disease, disorder, or condition and/or a neurological disease, disorder, or condition in a subject in need of therapy, comprising administering a therapeutically effective amount of a compound of general Formula I, or a pharmaceutically acceptable salt thereof to the subject.

Therefore, in some embodiments, the disease, disorder, or condition that is treated by activation of a serotonin receptor is a central nervous system (CNS) disease, disorder, or condition, and/or a neurological disease, disorder, or condition. Accordingly, the present disclosure also includes a method of treating a CNS disease, disorder, or condition, and/or a neurological disease, disorder or condition comprising administering a therapeutically effective amount of one or more compounds of the present disclosure or pharmaceutically acceptable salts thereof to a subject in need thereof. The present disclosure also includes a use of one or more compounds of Formula I or pharmaceutically acceptable salts thereof for treatment of a CNS disease, disorder, or condition, and/or a neurological disease, disorder, or condition, as well as a use of one or more compounds of the present disclosure for the preparation of a medicament for treatment of a CNS disease, disorder, or condition, and/or a neurological disease, disorder, or condition. The present disclosure further includes one or more compounds of the disclosure or pharmaceutically acceptable salts thereof for use in treating a CNS disease, disorder, or condition, and/or a neurological disease, disorder, or condition.

In some embodiments the CNS disease, disorder or condition, and/or neurological disease, disorder or condition is selected from neurological diseases including neurodevelopmental diseases and neurodegenerative diseases such as Alzheimer's disease; presenile dementia; senile dementia; vascular dementia; Lewy body dementia; cognitive impairment, Parkinson's disease and Parkinsonian related disorders such as Parkinson dementia, corticobasal degeneration, and supranuclear palsy; epilepsy; CNS trauma; CNS infections; CNS inflammation; stroke; multiple sclerosis; Huntington's disease; mitochondrial disorders; Fragile X syndrome; Angelman syndrome; hereditary ataxias; neurotological and eye movement disorders; neurodegenerative diseases of the retina amyotrophic lateral sclerosis; tardive dyskinesias; hyperkinetic disorders; attention deficit hyperactivity disorder and attention deficit disorders; restless leg syndrome; Tourette's syndrome; schizophrenia; autism spectrum disorders; tuberous sclerosis; Rett syndrome; cerebral palsy; disorders of the reward system including eating disorders such as anorexia nervosa (“AN”) and bulimia nervosa (“BN”); and binge eating disorder (“BED”), trichotillomania, dermotillomania, nail biting; migraine; fibromyalgia; and peripheral neuropathy of any etiology, and combinations thereof.

In some embodiments, the subject is a mammal. In another embodiment, the subject is a human. In some embodiments, the subject is a non-human animal. In some embodiments, the subject is a canine. In some embodiments, the subject is a feline. Accordingly, the compounds, methods, and uses of the present disclosure are directed to both human and veterinary diseases, disorders, and conditions.

In some embodiments, the compounds of the disclosure are useful for treating behavioral problems in subjects that are felines or canines.

The present disclosure also includes a use of one or more compounds of the disclosure for treatment of a behavioral problem in a non-human subject, as well as a use of one or more compounds of the disclosure for the preparation of a medicament for treatment of a behavioral problem in a non-human subject. The present disclosure further includes one or more compounds of the disclosure for use in treating a behavioral problem in a non-human subject.

In some embodiments, the behavioral problems in a non-human subject are selected from, but are not limited to, anxiety, fear, stress, sleep disturbances, cognitive dysfunction, aggression, excessive noise making, scratching, biting, and a combination thereof.

In some embodiments, the non-human subject is a canine. In some embodiments, the non-human subject is a feline.

In some embodiments, methods include treating a psychiatric disorder by administering to a subject in need thereof a pharmaceutical composition including about 0.01 mg to about 400 mg of a compound disclosed herein. In some embodiments, doses may be, e.g., in the range of about 0.01 to about 300 mg, or about 0.01 to about 250 mg, or about 0.01 to about 200 mg, or about 0.01 to 150 mg, or about 0.01 to about 100 mg, or about 0.01 to about 75 mg, or about 0.01 to about 50 mg, or about 0.01 to about 25 mg, or about 0.01 to about 20 mg, or about 0.01 to about 5 mg, or about 0.01 to about 10 mg, or about 0.01 to about 5 mg, or about 0.01 to about 1 mg, or about 0.01 to about 0.1 mg, or about 0.1 to about 300 mg, or about 0.1 to about 250 mg, or about 0.1 to about 200 mg, or about 0.1 to 150 mg, or about 0.1 to about 100 mg, or about 0.1 to about 75 mg, or about 0.1 to about 50 mg, or about 0.1 to about 25 mg, or about 0.1 to about 20 mg, or about 0.1 to about 5 mg, or about 0.1 to about 10 mg, or about 0.1 to about 5 mg, or about 0.1 to about 1 mg, or about 10 to about 300 mg, or about 10 to about 250 mg, or about 10 to about 200 mg, or about 10 to about 150 mg, or about 10 to about 100 mg, or about 10 to about 50 mg, or about 10 to about 25 mg, or about 10 to about 15 mg, or about 20 to about 300 mg, or about 20 to about 250 mg, or about 20 to about 200 mg, or about 20 to about 150 mg, or about 20 to about 100 mg, or about 20 to about 50 mg, or about 50 to about 300 mg, or about 50 to about 250 mg, or about 50 to about 200 mg, or about 50 to about 150 mg, or about 50 to about 100 mg, or about 100 mg to about 300 mg, or about 100 to about 250 mg, or about 100 to about 200 mg. Specific examples of doses include about 0.01 mg, 0.025 mg, 0.05 mg, 0.075 mg, 0.1 mg, 0.15 mg, 0.25 mg, about 0.5 mg, about 0.75 mg, about 1 mg, about 1.25 mg, about 1.5 mg, about 1.75 mg, about 2.0 mg, about 2.5 mg, about 3.0 mg, about 3.5 mg, about 4.0 mg, about 4.5 mg, about 5 mg, about 10 mg, about 15 mg, about 20 mg, about 25 mg, about 30 mg, about 35 mg, about 40 mg, about 45 mg, about 50 mg, about 75 mg, about 100 mg, about 125 mg, about 150 mg, about 175 mg, about 200 mg, about 225 mg, about 250 mg, about 275 mg, about 300 mg, and about 400 mg.

In some embodiments, dosages may include amounts of a compound of Formula I or a pharmaceutically acceptable salt thereof in the range of e.g., about 1 mg to about 200 mg, or about 1 mg to about 100 mg, or about 1 mg to about 50 mg, or about 1 mg to about 40 mg, or about 1 mg to about 30 mg, or about 1 mg to 20 mg, or about 1 mg to about 15 mg, or about 0.1 mg to about 10 mg, or about 0.1 mg to about 15 mg, or about 1.5 mg to about 12.5 mg, or about 2 mg to about 10 mg, or about 0.01 mg to about 1 mg, or about 0.01 mg to about 0.1 mg. Specific examples include doses of about 0.01 mg, about 0.025 mg, about 0.05 mg, about 0.075 mg, about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 1.25 mg, about 1.50 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 15 mg, about 20 mg, about 25 mg, about 30 mg, about 35 mg, about 40 mg, about 45 mg, about 50 mg, about 60 mg, about 70 mg, about 75 mg, about 80 mg, about 90 mg, about 100 mg, about 125 mg, about 150 mg, and about 200 mg.

Typically, dosages of a compound of Formula I or a pharmaceutically acceptable salt thereof is administered at a dosing interval of once, twice, three or four times daily, or every other day, or every three days, or once weekly, or once a month, or every other month, or every 3 months, or every 6 months, or once a year to a patient in need thereof. In some embodiments, the dosage is, e.g., about 0.01 to about 400 mg/dosing interval, or about 0.01 to about 300 mg/dosing interval, or about 0.01 to about 250 mg/dosing interval, or about 0.01 to about 200 mg/dosing interval, for example, about 300 mg/dosing interval, or about 250 mg/dosing interval, or 200 mg/dosing interval, or about 150 mg/dosing interval, or about 100 mg/dosing interval, or about 75 mg/dosing interval, or about 50 mg/dosing interval, or about 25 mg/dosing interval, or about 20 mg/dosing interval, or about 10 mg/dosing interval, or about 5 mg/dosing interval, or about 1 mg/dosing interval, or about 0.5 mg/dosing interval, or about 0.1 mg/dosing interval, or about 0.05 mg/dosing interval, or about 0.01 mg/dosing interval.

In some embodiments, pharmaceutical compositions for parenteral or inhalation, e.g., a spray or mist of a compound of Formula I or a pharmaceutically acceptable salt thereof, include a concentration of about 0.001 mg/mL to about 500 mg/mL. In some embodiments, the compositions include a compound disclosed herein or a pharmaceutically acceptable salt thereof, at a concentration of, e.g., about 0.05 mg/mL to about 50 mg/mL, about 0.05 mg/mL to about 100 mg/mL, about 0.005 mg/mL to about 500 mg/mL, about 0.1 mg/mL to about 50 mg/mL, about 0.1 mg/mL to about 10 mg/mL, about 0.05 mg/mL to about 25 mg/mL, about 0.05 mg/mL to about 10 mg/mL, about 0.05 mg/mL to about 5 mg/mL, about 0.05 mg/mL to about 1 mg/mL, about 0.001 mg/mL to about 1 mg/mL, about 0.001 mg/mL to about 0.1 mg/mL, about 0.001 mg/mL to about 0.01 mg/mL.

In some embodiments, the composition includes a compound disclosed herein or a pharmaceutically acceptable salt thereof, at a concentration of, e.g., about 0.001 mg/mL to about 0.01 mg/mL, about 0.01 mg/mL to about 0.1 mg/mL, about 0.1 mg/mL to about 1 mg/mL, about 0.05 mg/mL to about 15 mg/mL, about 0.5 mg/mL to about 10 mg/mL, about 0.25 mg/mL to about 5 mg/mL, about 0.5 mg/mL to about 7 mg/mL, about 1 mg/mL to about 10 mg/mL, about 5 mg/mL to about 10 mg/mL, about 5 mg/mL to about 15 mg/mL, about 5 mg/mL to about 25 mg/mL, about 5 mg/mL to about 50 mg/mL, or about 10 mg/mL to about 100 mg/mL. In some embodiments, the pharmaceutical compositions are formulated as a total volume of about, e.g., 0.1 mL, 0.2 mL, 0.5 mL, 1 mL, 2 mL, 5 mL, 10 mL, 20 mL, 25 mL, 50 mL, 100 mL, 200 mL, 250 mL, or 500 mL.

Typically, dosages may be administered to a subject once, twice, three, or four times daily, every other day, every three days, twice weekly, once weekly, twice monthly, once monthly, every other month, every 3 months, twice yearly, or yearly. In some embodiments, a compound disclosed herein is administered to a subject once in the morning, or once in the evening. In some embodiments, a compound disclosed herein is administered to a subject once in the morning, and once in the evening. In some embodiments, a disclosed herein is administered to a subject three times a day (e.g., at breakfast, lunch, and dinner), at a dose, e.g., of 50 mg/administration (e.g., 150 mg/day).

In some embodiments, a compound disclosed herein is administered to a subject at a dose of 0.01 mg/day in one or more doses. In some embodiments, a compound disclosed herein is administered to a subject at a dose of 0.1 mg/day in one or more doses. In some embodiments, a compound disclosed herein is administered to a subject at a dose of 0.5 mg/day in one or more doses. In some embodiments, a compound disclosed herein is administered to a subject at a dose of 1 mg/day in one or more doses. In some embodiments, a compound disclosed herein is administered to a subject at a dose of 2.5 mg/day in one or more doses. In some embodiments, a compound disclosed herein is administered to a subject at a dose of 5 mg/day in one or more doses. In some embodiments, a compound disclosed herein is administered to a subject at a dose of 10 mg/day in one or more doses. In some embodiments, a compound disclosed herein is administered to a subject at a dose of 15 mg/day in one or more doses. In some embodiments, a compound disclosed herein is administered to a subject at a dose of 25 mg/day in one or more doses. In some embodiments, a compound disclosed herein is to be administered to a subject at a dose of 50 mg/day in one or more doses. In some embodiments, a compound disclosed herein is administered to a subject at a dose of 75 mg/day in one or more doses. In some embodiments, a compound disclosed herein is administered to a subject at a dose of 100 mg/day in one or more doses. In some embodiments, a compound disclosed herein is administered to a subject at a dose of 150 mg/day in one or more doses. In some embodiments, a compound disclosed herein is administered to a subject at a dose of 200 mg/day in one or more doses. In some embodiments, a compound disclosed herein is administered to a subject at a dose of 250 mg/day in one or more doses.

In some embodiments, the dosage of a compound disclosed herein is 0.0001-10 mg/kg, 0.0001-0.01 mg/kg, 0.001-1 mg/kg, 0.01-1 mg/kg, 0.5-5 mg/kg, or 0.5-10 mg/kg once, twice, three times, or four times daily. For example, in some embodiments, the dosage is 0.0001 mg/kg, 0.001 mg/kg, 0.01 mg/kg, 0.025 mg/kg, 0.05 mg/kg, 0.1 mg/kg, 0.25 mg/kg, 0.5 mg/kg, 1 mg/kg, 2.5 mg/kg, 5 mg/kg, 7.5 mg/kg, or 10 mg/kg once, twice, three times, or four times daily. In some embodiments, a subject is administered a total daily dose of 0.01 mg to 500 mg of a compound disclosed herein once, twice, three times, or four times daily. In some embodiments, the total amount administered to a subject in 24-hour period is, e.g., 0.01 mg, 0.05 mg, 0.1 mg, 0.25 mg, 0.5 mg, 1 mg, 2 mg, 3 mg, 5 mg, 10 mg, 20 mg, 25 mg, 30 mg, 35 mg, 40 mg, 45 mg, 50 mg, 60 mg, 75 mg, 80 mg, 90 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg. In some embodiments, the subject may be started at a low dose and the dosage is escalated. In some embodiments, the subject may be started at a high dose and the dosage is decreased.

In some embodiments, a compound or composition disclosed herein may be administered at specified intervals. For example, during treatment a patient may be administered a compound or composition at intervals of every, e.g., 1 year, 6 months, 120 days, 90 days, 60 days, 30 days, 14 days, 7 days, 3 days, 24 hours, 12 hours, 8 hours, 6 hours, 5 hours, 4 hours, 3 hours, 2.5 hours, 2.25 hours, 2 hours, 1.75 hours, 1.5 hours, 1.25 hours, 1 hour, 0.75 hour, 0.5 hour, or 0.25 hour.

In some embodiments, the compound is administered under the supervision of a healthcare provider.

In some embodiments, the compound is administered in a clinic specializing in the administration of psychedelic medicines.

In some embodiments, the compound is administered at home or otherwise away from the supervision of a healthcare provider.

In some embodiments, a compound disclosed herein is in the form of a pharmaceutically acceptable salt thereof.

In some embodiments, a pharmaceutical composition comprises one or more of the compounds disclosed herein.

In some embodiments, a salt of the compound disclosed herein is used in any of the methods, uses, or compositions.

In some embodiments, a pharmaceutically acceptable salt of the compound disclosed herein is used in any of the methods, uses, or compositions.

The compounds of Formula I are prepared by art-recognized procedures. The synthesis of compounds of Formula I are illustrated by the following examples. The syntheses utilize types of organic reactions, some of which are described in standard organic chemistry textbooks, including “Advanced Organic Chemistry,” Fifth Edition, by Michael Smith and Jerry March, John Wiley & Sons, Inc., New York (2001). It will be evident to one skilled in this art that the synthetic schemes in the examples presented below may be modified to provide additional related compounds. The following non-limiting examples further illustrate the subject matter in the present disclosure.

The following scheme illustrates exemplary procedures for preparing compounds of Formula I.

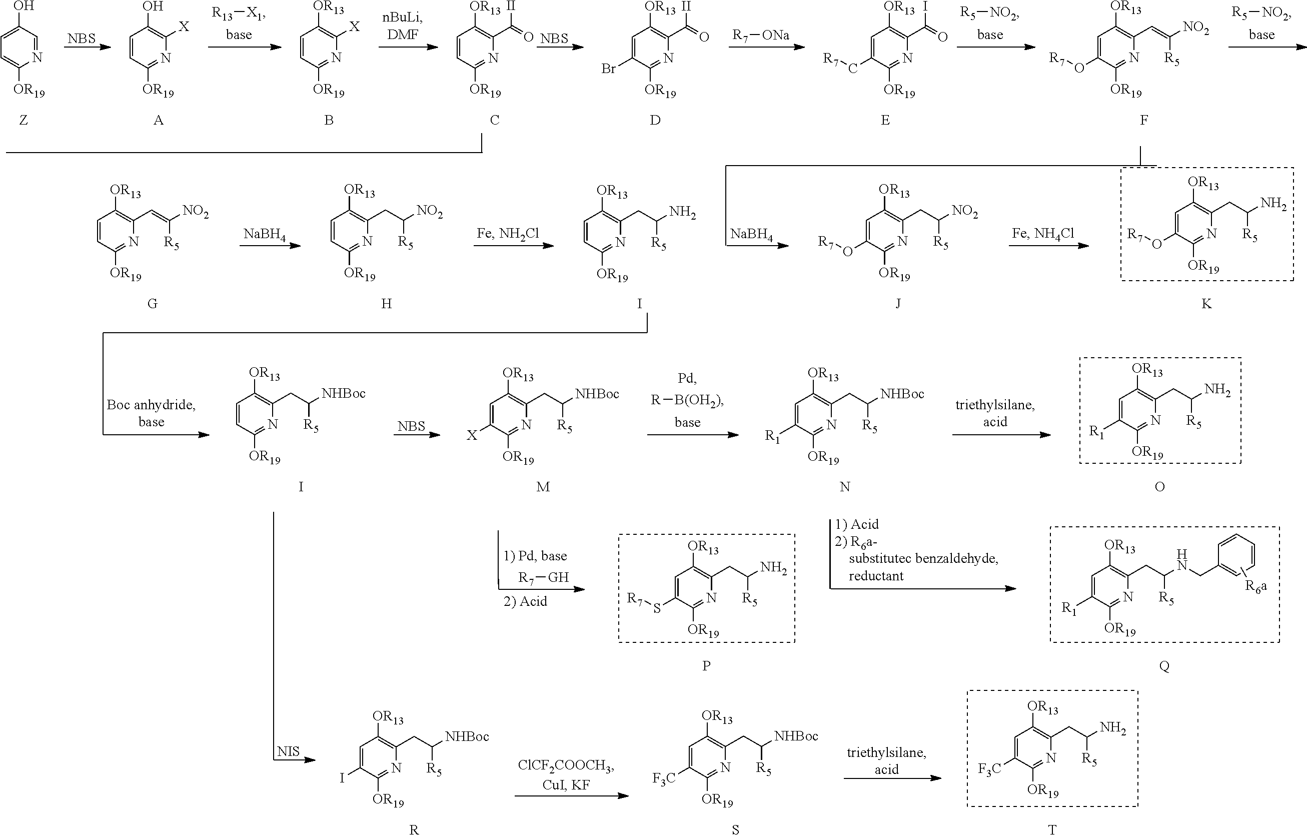

As shown in the above Scheme, a pyridine-3-ol compound (Compound Z) is halogenated, e.g., brominated with a halogenating reagent, such as N-bromosuccinimide (NBS), bromine, 1,3-dibromo5,5-dimethylhydantoin, tetrabutylammonium tribromide, N-bromo saccharin pyridinium bromide perbromide, or benzyltrimethylammonium tribromide, and the like, in a solvent such as acetonitrile, acetonitrile-water, dioxane, or dioxane-water, and the like, at a temperature ranging from about 25° C. to about 100° C., optimally at the reflux temperature of the chosen solvent, to form the halogenated derivative (Compound A), wherein X is halogen, such as Br, in the reaction shown. The 2-halo-3-hydroxypyridine is treated with a base, such as sodium or potassium carbonate, sodium or potassium bicarbonate, sodium or potassium hydroxide, or a sodium alkoxide, such as sodium methoxide, and the like in a neutral solvent such as DMF, DMA, THF, or dimethyl acetamide, and the like, and reacted with a halide R₁₃X₁, such as methyl iodide, and the like, under standard ether forming conditions, in a solvent at a temperature ranging from about 25° C. and about 100° C., optimally about 60° C. to produce Compound B. Compound B is then formlyated under conditions known in the art. For example, the halide (Compound B) is reacted with an organolithium reagent such as n-butyl or sec-butyl lithium, and the like, with a N,N-disubstituted formamide, such as dimethylformamide, and the like, in a solvent such as THF, ether, or similar solvents, at a temperature ranging from about −78° C. to about 0° C., under Bouveault aldehyde synthesis conditions to form an aldehyde (Compound C). Aldehyde Compound C may also be made by a three-step sequence involving palladium catalyzed coupling of a vinyl boronate in a suitable solvent, such as THF or dioxane, and the like, with a suitable base such as an alkyl metal carbonate or bicarbonate, and the like at about room temperature up to about the reflux temperature of the solvent, to afford an olefin, which can then be hydroxylated through the action of catalytic osmium tetroxide with a catalyst such as periodate at room temperature in a solvent such as THF-water or acetone-water to afford a diol, which is then cleaved through the action of periodate to afford the aldehyde Compound C. In one scenario, compound C is halogenated, e.g., brominated with a halogenating reagent, such as N-bromosuccinimide (NBS), bromine, 1,3-dibromo-5,5-dimethylhydantoin, tetrabutylammonium tribromide, N-bromo saccharin pyridinium bromide perbromide, or benzyltrimethylammonium tribromide, and the like, in a solvent such as acetonitrile, acetonitrile-water, dioxane, or dioxane-water, and the like, at a temperature ranging from about 25° C. to about 100° C., optimally at about the reflux temperature of the chosen solvent, to form the halogenated derivative (Compound D). Compound D is reacted with a sodium or potassium alkoxide, such as R₇O⁻ salt, wherein R₇ is defined herein, under palladium or copper catalysis in a suitable solvent such as DMF, THF, DMSO, or DMA, and the like, at a temperature from about 75° C. to about 150° C., to form the ether (Compound E). Compound E is reacted with an organic nitro compound, such as R₅CH₂NO₂, wherein R₅ is as defined herein, in the presence of a base, such as alkali metal hydroxides, carbonates, bicarbonates, or alkoxides, alkaline earth metal hydroxides, aluminum ethoxides; organic bases such as primary, secondary, and tertiary amines; or ammonium acetate or other salts and the like, under Henry reaction conditions, using the nitroalkane as the solvent or through the use of solvents such as THF, diethyl ether, acetonitrile, or methylene chloride, and the like at temperatures ranging from about room temperature up to about the reflux point of the solvent, to form Compound F. The carbon-carbon double bond in Compound F is reduced with a reducing reagent such as sodium borohydride, lithium aluminum hydride, hydrogen gas with nickel, hydrogen in the presence of palladium or platinum catalysis, or other reducing agents known in the art in a solvent such as methanol, ethanol, ethyl acetate, THF, or similar solvents, at room temperature to the reflux point of the solvent, to produce Compound J. If not reduced in the previous step, the nitro group of Compound J is reduced with a reducing reagent, such as Fe, Zn, or Sn, in the presence of acid or Raney Nickel and hydrogen gas in a suitable solvent such as water, methanol, or ethanol, to form the corresponding primary amine of Compound K.

Compound C formed herein above is an intermediate in other reactions. For example, it is reacted with an organic nitro compound, such as R₅CH₂NO₂, wherein R₅ is as defined herein, in the presence of a base, such as alkali metal hydroxides, carbonates, bicarbonates, or alkoxides, alkaline earth metal hydroxides, aluminum ethoxides, organic bases such as primary, secondary, and tertiary amines, or ammonium acetate or other salts and the like, under Henry reaction conditions using the nitroalkane as the solvent or through the use of solvents such as THF, diethyl ether, acetonitrile, or methylene chloride, and the like at temperatures ranging from about room temperature up to about the reflux point of the solvent, to form Compound G. The carbon-carbon double bond in Compound G is reduced with a carbon-carbon double bond reducing reagent such as sodium borohydride, lithium aluminum hydride, hydrogen gas with nickel, hydrogen with palladium or platinum catalysis, or other reducing agents known in the art in a solvent such as methanol, ethanol, ethyl acetate, THF, or similar solvents, at room temperature to the reflux point of the solvent, to produce compound H. If not reduced in the previous step, the nitro group is reduced to the corresponding primary amine with a nitro reducing reagent, such as Fe, Zn, or Sn, in the presence of acid or Raney Nickel and hydrogen gas in a suitable solvent such as water, methanol, or ethanol, to form the corresponding amine Compound I. The amine is protected with an amine protecting group known in the art, such as converting the amine to a carbamate, e.g., a t-butyl carbamate (Boc group) with Boc anhydride or Boc-ON or a benzyl carbamate (Cbz) with Cbz-Cl, using a solvent such as THF, dioxane, or acetonitrile alone or in combination with water as a co-solvent. Alternatively, methylene chloride, toluene or other solvents may be used with or without water and with or without a base such as aq. sodium bicarbonate or sodium carbonate, triethylamine, DMAP, or Hunig's base. The reaction is conveniently run at room temperature up to the reflux point of the solvent to provide Compound L. Compound L is converted to Compound O in which the R₁ group, as defined herein, is bonded to the pyridine ring. This may be effected by reacting Compound L with a halogenating reagent, such as NBS, bromine, 1,3-dibromo5,5-dimethylhydantoin, tetrabutylammonium tribromide, N-bromo saccharin, pyridinium bromide perbromide, or benzyltrimethylammonium tribromide, and the like in a solvent such as acetonitrile, acetonitrile-water, dioxane, or dioxane-water, and the like, at a temperature ranging from about 25° C. to about 100° C., optimally at about the reflux temperature of the chosen solvent, to form compound M. Compound M is subjected to oxidative carbon-carbon coupling by reactions known in the art, such as by reacting Compound M with organometallic reagents, such as a copper lithium complex, e.g., (R₁)₂CuLi, wherein R₁ is as defined herein, in a solvent such as THF or ether, and the like at a temperature ranging from about room temperature down to about −78° C., or by using a variation of the Suzuki coupling reaction by reacting Compound M with a boronic acid, R₁B(OH)₂, in the presence of a palladium catalyst, such as Pd(dppf)Cl₂, Pd tetrakistriphenylphosphine, or similar ligated palladium (0) or palladium (II) complexes in the presence of a base such as cesium, sodium, or potassium carbonate, or sodium or potassium bicarbonate, in a solvent such as toluene-water, THF-water, or dioxane-water, and the like at a temperature of about room temperature up to about the reflux point of the solvent, to produce Compound N. The protecting group, t-Boc, is removed by reactions known in the art, such as by treatment under acidic conditions with TFA, HCl, or HBr, and the like, with or without triethyl silane or anisole, in a solvent such as methylene chloride, acetonitrile, toluene, or ethyl acetate, and the like at room temperature or below. If a Cbz group was used, then deprotection is conducted in ethyl acetate using a palladium charcoal or palladium hydroxide catalyst under a hydrogen atmosphere. Alternatively, the Cbz group may be removed using a solution of HBr in acetic acid. Removal of the protecting group in both cases leads to formation of Compound O. Alternatively, Compound M may be deprotected directly to provide the halogenated derivative of the amine (e.g., the bromide).

Compound Q is formed from Compound N. The amine protecting group is removed by reactions described above and which are known in the art. In this case, since the amine protecting group is Boc, it is removed by acid, or if it is Cbz, it is removed by hydrogenation over palladium catalysis, and the resulting primary amine is then reacted with an R₆ₐ substituted benzaldehyde under reductive amination conditions, with or without the removal of water, to form an imine, the C═N bond of which is reduced by a reducing agent known in the art, such as by hydrogenation reactions, or by using, zinc and HCl, or sodium cyanoborohydride, sodium triacetoxyborohydride, or sodium borohydride in a solvent such a methylene chloride, acetonitrile, 1,2-dichloroethane, or similar inert solvents at room temperature, to afford compound Q.

Compound P is formed from Compound M. R₇SH, wherein R₇ is as defined herein, in the presence of a base, such as a hydroxide base, and a transition metal, such as palladium, forms a thiolate nucleophile, which reacts with Compound M to form an amine protected thioether. The amine protecting group is then removed by techniques known in the art and as described above. (e.g., Boc is removed by reacting the resulting product with acid, to form Compound P). Halogenation of Compound L, such as with N-iodosuccinimide (NIS) under halogenating conditions, in a solvent such as acetonitrile, acetonitrile-water, dioxane, or dioxane-water at a temperature ranging from about 25° C. to about 100° C., optimally at about the reflux temperature of the chosen solvent, forms the iodinated product Compound R. Compound R is reacted with a trifluormethylating reagent, such as copper(I) chlorodifluoroacetate complexes formed by reacting CuI with ClCF₂COOCH₃ and KF to form Compound S. Removal of the amine protecting group by techniques know in the art and as described above affords the amine product T.

One of ordinary skill in the art can prepare the compounds of Formula I using known techniques combined with the teachings herein. Once a functional group is placed at a position, one of ordinary skill in the art can prepare compounds wherein R₁, R₂, R₃, R₄, R₅, and R₆ are as defined herein. For example, with respect to R₁ definitions at the 5-position of the pyridine derivative, the compounds where R₁ is hydrogen may be prepared by deprotection of Compound L. Alternatively, they may be prepared by converting the OR₇ group, such as on Compound J, to a mesylate or triflate, and then reacting the resulting product with a nickel catalyst and hydrogen in DMF, and then removing the amine protecting group. Alternatively, a 5-halo substituent, such as the one prepared in Compound M, is dehalogenated by hydrogenation in the presence of transition metal catalysts, such as palladium or nickel. The alkyl, alkenyl, alkynyl, cycloalkyl, cycloalkyl alkyl, aryl, heteroaryl, arylalkyl, heteroarylalkyl, heterocyclic, or heterocyclic alkyl substituents may be prepared by carbon-carbon coupling reactions known in the art, such as shown in Examples 1, 2, 4, 5, 7, and 8, below. Compounds wherein R₁ is CN can be prepared by techniques known in the art, such as by treatment of the 5-OR₇ substituted pyridine, wherein R₇ is H, with cyanogen halides in the presence of a base or by reaction of a pyridine halide, such as Compound M, with anhydrous CuCN under Rosenmund-von Braun reaction conditions. Reduction of the cyano group with reducing agents known in the art, such as LiAlH₄, BH₃-Me₂S, or NaBH4 in an alcoholic solvent and CoCl₂ produces a primary amine. Secondary and tertiary amines can be prepared by reacting the primary amine thus formed with R₈X₂, wherein X₂ is a halide to form the secondary amine and reacting the secondary amine with R₉X₂, wherein R₈ and R₉ are as defined herein to form the tertiary amine. Alternatively, the primary amine may be reacted with aldehydes under reductive amination conditions to afford secondary or tertiary amines. Nitration of the pyridine compound is effected by techniques known in the art such as reacting 5-bromopyridine derivative, such as Compound M with nitrous acid or sodium nitrite in acetic acid under Zincke nitration conditions. Alternatively. Compound L may be nitrated directly, for example, with HNO₃ in various acids, such as acetic acid, sulfuric acid, or triflic acid, to provide the 5-nitropyridine derivatives. These may be reduced with Fe, Zn, or Sn under conditions known in the art to provide the 5-aminopyridine derivatives, which may in turn be substituted with alkyl groups via alkylation or reductive amination, as described above.

Using the techniques described herein, the 3-position substituents, i.e., R₃, can be prepared as described herein. As shown by the scheme hereinabove and the examples hereinbelow, pyridine derivatives wherein R₃ is —OR₁₃ can be prepared.

The 3-hydroxypyridine derivatives, such as compound M wherein R₁₃ is H, can be converted to the corresponding bromide via boronate esters intermediates by reacting the 2-hydroxypridine derivative with Tf₂O in pyridine and reacting the product with bis(pinacolato)diboron in PdCl₂(dppf) in NEt₃ and reacting the resulting product with cuprous bromide in H₂O(I/MeOH 1:1 mixture under reflux. For example, with respect to R₃ definitions at the 3-position of the pyridine, the compounds with R₃ being hydrogen can be prepared, by converting the —OR₁₃ group, such as on Compound N, to a mesylate or triflate, and then reacting the resulting product with hydrogen and a nickel catalyst in DMF, and then removing the amine protecting group. Alternatively, a 3-halo-substituted derivative is dehalogenated by hydrogenation over a transition metal catalyst, such as palladium or nickel. The alkyl, alkenyl, alkynyl, cycloalkyl, aryl, heteroaryl, arylalkyl, and heteroarylalkyl, cycloalkyl alkyl, heterocyclic, or heterocyclic alkyl substituents at this position are prepared by carbon-carbon coupling reactions known in the art, such as shown in Examples 1, 2, 4, 5, 7, and 8, below. Compounds wherein R₃ is CN can be prepared by techniques known in the art, such as by treatment of the 3-OH substituted pyridine, wherein R₃ is OH, with cyanogen halides in the presence of a base or by reaction of a pyridine halide, wherein R₃ is a halide, such as I or Br, with anhydrous CuCN under Rosenmund-von Braun reaction conditions. Reduction of the cyano group with reducing agents known in the art, such as LiAlH₄, BH₃-Me₂S, or NaBH₄, and the like in an alcoholic solvent and CoCl₂ produces a primary amine. Secondary and tertiary amines can be prepared by reacting the primary amine thus formed with R₁₄X₂, wherein X₂ is a halide, to form the secondary amine and reacting the secondary amine with R₁₅X₂, wherein R₁₄ and R₁₅ are as defined herein, to form the tertiary amine. Alternatively, the primary amine may be reacted with aldehydes under reductive amination conditions to afford secondary or tertiary amines. Esterification of a 3-hydroxypyridene derivative with an acid chloride under esterification conditions affords the ester. To form the 3-carboxy pyridine derivatives, the starting material is 3-carboxy-6-hydroxy pyridine, and the carboxy group is then protected by a carboxy protecting group known in the art, and then following the reactions in the Scheme above. However, prior to converting the nitro group to an amine or removing the amine protecting group, the carboxy protecting group is removed. The carboxylic acid thus formed is reacted with alcohols or amines under esterification or amide forming conditions to form esters or amides, respectively.

Similarly, the 6-hydroxypyridine derivatives, such as compound M wherein R₁₉ is H, can be converted to derivatives bearing alternative substituents at this position according to procedures analogous to those described above for transformation of the 3-hydroxypyridine derivatives.

The following scheme illustrates additional exemplary procedures for preparing compounds of Formula I.

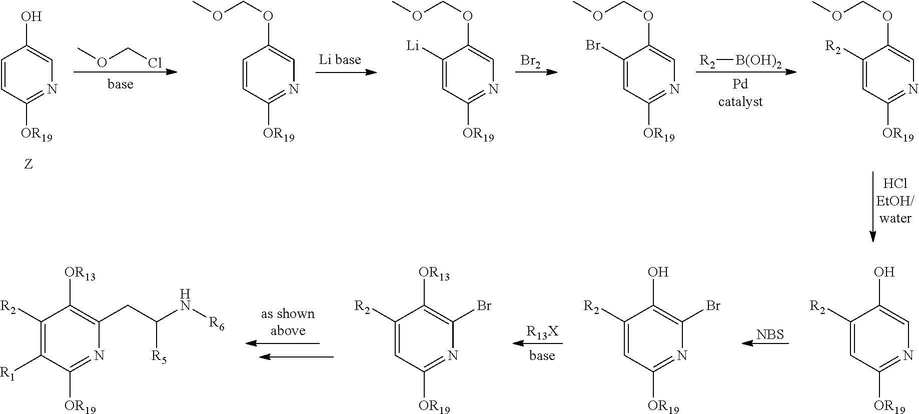

As shown in the above scheme, 4-substituted pyridines and the resulting compounds of the invention where R₂ is other than H, may be prepared from common intermediates, such as Compound Z, after addition of an anion directing group, such as a methoxymethyl or tetrahydropyran, using well known methods from the literature. Formation of the anion is accomplished with a strong base such as lithium diisopropylamide, lithium hexamethyldisilazane, lithium tetramethylpiperidide, or butyllithium or methyllithium with TMEDA, in a suitable solvent such as THF or other aprotic ethers, at a temperature from −78° C. to 0° C. The anion may be reacted with a halogenating agent such as bromine or NBS, as well as NCS, N-iodosuccinimide, or a fluorinating agent. The anion may also be reacted with DMF to form an aldehyde. The resulting bromide may, for example, be elaborated to introduce an alkyl or aryl substituent using palladium, nickel, or copper catalysis under Suzuki conditions with boron reagents, as well as related Kumada, Molander, or Weix coupling protocols and other well-known forms of coupling that involve metal insertion and reductive elimination. The bromide may also be subjected to many other forms of elaboration by well-known methods familiar in the art. The anion directing group can be readily removed through hydrolysis and the resulting phenol can be alkylated as described above. Further elaboration to additional compounds of the disclosure is accomplished as described above to afford the desired pyridylalkyl amines of Formula I.

The R₅ substituent is attached to an organic moiety that connects to the main chain by carbon-carbon coupling reactions known in the art. Some are illustrated in the scheme and in the exemplification.

The R₆ substituent is added to the chain by reacting with the free amino group by alkylation or reductive amination reactions, as described above.

In all of these reactions, if necessary, if there is a moiety on the compound that is reactive under the necessary reaction conditions, then that group is protected with a protecting group that is stable under the conditions to be used.

### Example 1: Preparation of 1-(3,6-dimethoxy-5-pentylpyridin-2-yl)butan-2-amine (1)

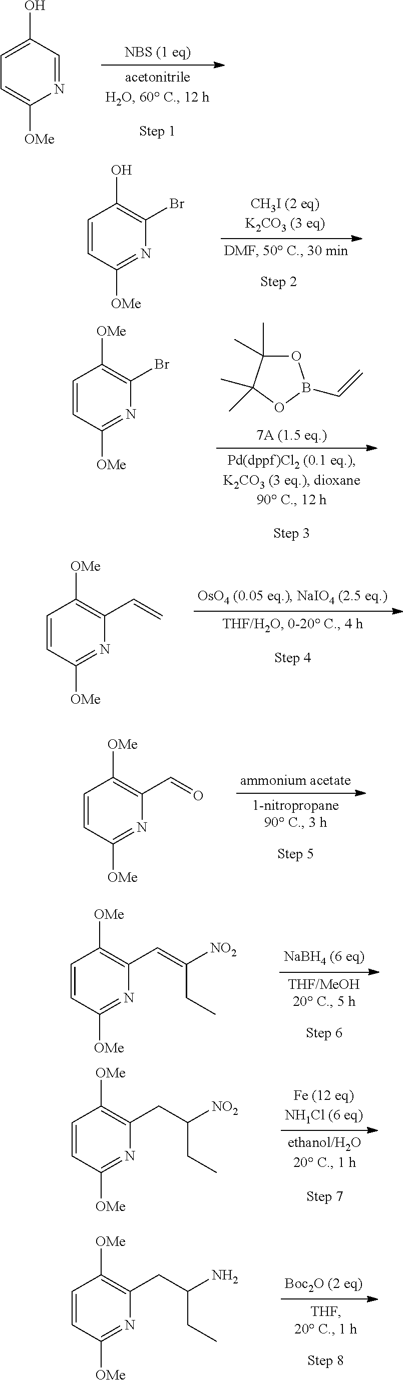

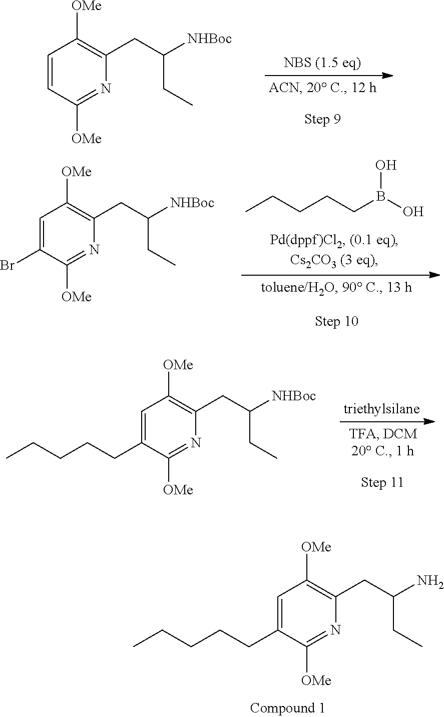

### Step 1: Preparation of 2-bromo-6-methoxypyridin-3-ol

To a solution of 6-methoxypyridin-3-ol (25 g, 199.80 mmol, 1 eq) in acetonitrile (250 mL) and water (25 mL) was added NBS (35.56 g, 199.80 mmol, 1 eq). The mixture was stirred at 60° C. for 12 hours then diluted with water (30 mL) and extracted with ethyl acetate (20 mL×3). The combined organic layers were washed with brine (20 mL), dried over Na₂SO₄, filtered, and concentrated under reduced pressure. The residue was purified by column chromatography (SiO₂, petroleum ether/ethyl acetate gradient, 50:1 to 3:1). Compound 2-bromo-6-methoxypyridin-3-ol (33 g, 161.75 mmol, 80.95% yield) was obtained as a white solid. ¹H NMR (400 MHz, CHLOROFORM-d) δ ppm 3.89 (s, 3H), 5.04 (br s, 1H), 6.66 (d, J=8.66 Hz, 1H), 7.27 (d, J=8.66 Hz, 1H).

### Step 2: Preparation of 2-bromo-3,6-dimethoxypyridine

To a solution of 2-bromo-6-methoxypyridin-3-ol (33 g, 161.75 mmol, 1 eq) in DMF (150 mL) was added K₂CO₃ (67.06 g, 485.24 mmol, 3 eq) and CH₃I (57.40 g, 404.37 mmol, 25.17 mL, 2.5 eq). The mixture was stirred at 50° C. for 0.5 hr then was diluted with water 100 mL and extracted with ethyl acetate (100 mL×3). The combined organic layers were washed with brine (80 mL), dried over Na₂SO₄, filtered, and concentrated under reduced pressure to give a residue. Compound 2-bromo-3,6-dimethoxypyridine (35 g, 160.52 mmol, 99.24% yield) was obtained as yellow oil. ¹H NMR (400 MHz, CHLOROFORM-d) δ ppm 3.87 (d, J=15.88 Hz, 6H), 6.67 (d, J=8.63 Hz, 1H), 7.20 (d, J=8.76 Hz, 1H).

### Step 3: Preparation of 3,6-dimethoxy-2-vinylpyridine

A mixture of 2-bromo-3,6-dimethoxypyridine (30 g, 137.58 mmol, 1 eq), 4,4,5,5-tetramethyl-2-vinyl-1,3,2-dioxaborolane (31.78 g, 206.38 mmol, 35.01 mL, 1.5 eq) and K₂CO₃ (57.05 g, 412.75 mmol, 3 eq) in water (10 mL) and dioxane (100 mL) was degassed and purged with N₂ (3×), and then the mixture was stirred at 0° C. for 10 min, then Pd(dppf)Cl₂ (10.07 g, 13.76 mmol, 0.1 eq) was added at 90° C. and stirred for 6 hr under N₂ atmosphere. On completion, the residue was diluted with 80 mL water and extracted with ethyl acetate (80 mL×3). The combined organic layers were washed with brine 80 mL, dried over Na₂SO₄, filtered, and concentrated under reduced pressure to give a residue that was purified by column chromatography (SiO₂, Petroleum ether/Ethyl acetate gradient, 50:1 to 10:1). Compound 3,6-dimethoxy-2-vinylpyridine (12.5 g, 75.67 mmol, 55.00% yield) was obtained as yellow oil. ¹H NMR (400 MHz, CHLOROFORM-d) δ ppm 3.81 (s, 3H), 3.94 (s, 3H), 5.43 (dd, J=10.67, 2.38 Hz, 1H), 6.38 (dd, J=17.25, 2.45 Hz, 1H), 6.63 (d, J=8.78 Hz, 1H), 7.13 (dd, J=17.25, 10.73 Hz, 1H), 7.21 (d, J=8.91 Hz, 1H).

### Step 4: Preparation of 3,6-dimethoxypicolinaldehyde

To a solution of 3,6-dimethoxy-2-vinylpyridine (11.5 g, 69.62 mmol, 1 eq) in THF (200 mL) and water (50 mL) was added OSO₄ (884.94 mg, 3.48 mmol, 180.60 μL, 0.05 eq), NaIO₄ (37.23 g, 174.04 mmol, 9.64 mL, 2.5 eq) and water (100 mL) at 0° C. The mixture was stirred at 25° C. for 4 h. The mixture was then extracted with ethyl acetate (80 mL×3). The combined organic layers were washed with brine 80 mL, dried over Na₂SO₄, filtered, and concentrated under reduced pressure to give a residue that was purified by column chromatography (SiO₂, Petroleum ether/Ethyl acetate gradient, 50:1 to 3:1). Compound 3,6-dimethoxypicolinaldehyde (6 g, 35.89 mmol, 51.56% yield) was obtained as a pink solid. ¹H NMR (400 MHz, CHLOROFORM-d) δ ppm 3.94 (d, J=1.10 Hz, 3H), 3.98 (d, J=1.34 Hz, 3H), 6.99 (dd, J=9.11, 1.16 Hz, 1H), 7.44 (d, J=9.05 Hz, 1H), 10.26 (s, 1H).

### Step 5: Preparation of (E)-3,6-dimethoxy-2-(2-nitrobut-1-en-1-yl)pyridine

To a solution of 3,6-dimethoxypicolinaldehyde (1 g, 5.98 mmol, 1 eq) in 1-nitropropane (3 mL) was added ammonium acetate (461.13 mg, 5.98 mmol, 1 eq). The mixture was stirred at 90° C. for 3 h. On completion, the mixture was diluted with water (5 mL) and extracted with ethyl acetate (3 mL×3). The combined organic layers were washed with brine (3 mL), dried over Na₂SO₄, filtered, and concentrated under reduced pressure to give a residue that was purified by column chromatography (SiO₂, Petroleum ether/Ethyl acetate gradient, 50:1 to 3:1). Compound (E)-3,6-dimethoxy-2-(2-nitrobut-1-en-1-yl)pyridine (540 mg, 2.27 mmol, 37.89% yield) was obtained as a yellow solid. ¹H NMR (400 MHz, CHLOROFORM-d) δ ppm 1.27 (t, J=7.28 Hz, 3H), 3.38 (q, J=7.36 Hz, 2H), 3.88 (s, 3H), 3.92 (s, 3H), 6.82 (d, J=9.03 Hz, 1H), 7.30 (d, J=9.03 Hz, 1H), 8.30 (s, 1H).

### Step 6: Preparation of 3,6-dimethoxy-2-(2-nitrobutyl)pyridine

To a solution of (E)-3,6-dimethoxy-2-(2-nitrobut-1-en-1-yl)pyridine (1 g, 4.20 mmol, 1 eq) in THF (10 mL) and MeOH (20 mL) was added NaBH₄ (952.80 mg, 25.18 mmol, 6 eq) at 0° C. and allowed to warm to 20° C. After 5 hr, the mixture was diluted with NH₄Cl (20 mL) and extracted with ethyl acetate (20 mL×3). The combined organic layers were washed with brine 15 mL, dried over Na₂SO₄, filtered, and concentrated under reduced pressure to give a residue. Compound 3,6-dimethoxy-2-(2-nitrobutyl)pyridine (1 g, 4.16 mmol, 99.16% yield) was obtained as a white solid.

### Step 7: Preparation of 1-(3,6-dimethoxypyridin-2-yl)butan-2-amine

To a solution of 3,6-dimethoxy-2-(2-nitrobutyl)pyridine (1 g, 4.16 mmol, 1 eq) in ethanol (9 mL) and water (3 mL) was added iron dust (2.79 g, 49.95 mmol, 12 eq) and NH₄Cl (1.78 g, 33.30 mmol, 8 eq). The mixture was stirred at 80° C. for 5 h. On completion, the mixture was filtered and concentrated under reduced pressure to give an aqueous solution. The pH was adjusted to 9 by saturated sodium bicarbonate, and the suspension was filtered to give a white solid. Compound 1-(3,6-dimethoxypyridin-2-yl)butan-2-amine (0.8 g, 3.80 mmol, 91.41% yield) was obtained as a white solid.

### Step 8: Preparation of tert-butyl (1-(3,6-dimethoxypyridin-2-yl)butan-2-yl)carbamate

To a solution of 1-(3,6-dimethoxypyridin-2-yl)butan-2-amine (0.8 g, 3.80 mmol, 1 eq) in THF (10 mL) was added Boc₂O (1.66 g, 7.61 mmol, 1.75 mL, 2 eq). The mixture was stirred at 20° C. for 0.5 h. On completion, the mixture was filtered and concentrated, and the residue was purified by column chromatography (SiO₂, Petroleum ether/Ethyl acetate gradient, 100:1 to 3:1) to afford tert-butyl (1-(3,6-dimethoxypyridin-2-yl)butan-2-yl)carbamate (880 mg, 2.84 mmol, 74.52% yield) as a white solid. ¹H NMR (400 MHz, CHLOROFORM-d) δ ppm 0.95 (t, J=7.47 Hz, 3H), 1.39 (s, 9H), 1.45-1.53 (m, 2H), 2.81-2.98 (m, 2H), 3.77-3.80 (m, 3H), 3.82-3.87 (m, 1H), 3.89 (s, 3H), 5.58 (br d, J=5.02 Hz, 1H), 6.57 (d, J=8.78 Hz, 1H), 7.17 (d, J=8.78 Hz, 1H).

### Step 9: Preparation of tert-butyl (1-(5-bromo-3,6-dimethoxypyridin-2-yl)butan-2-yl)carbamate

To a solution of tert-butyl (1-(3,6-dimethoxypyridin-2-yl)butan-2-yl)carbamate (880 mg, 2.84 mmol, 1 eq) in acetonitrile (10 mL) was added NBS (756.92 mg, 4.25 mmol, 1.5 eq). The mixture was stirred at 20° C. for 12 h. On completion, the mixture was diluted with water (5 mL) and extracted with ethyl acetate (10 mL×3). The combined organic layers were washed with brine (3 mL), dried over Na₂SO₄, filtered, and concentrated under reduced pressure to give a residue that was purified by column chromatography (SiO₂, Petroleum ether/Ethyl acetate gradient, 100:1 to 20:1) to afford tert-butyl (1-(5-bromo-3,6-dimethoxypyridin-2-yl)butan-2-yl)carbamate (780 mg, 2.00 mmol, 70.67% yield) as a white solid. ¹H NMR (400 MHz, CHLOROFORM-d) δ ppm 0.95 (t, J=7.45 Hz, 3H), 1.38 (s, 9H), 1.45-1.53 (m, 2H), 2.85 (d, J=6.36 Hz, 2H), 3.79 (s, 3H), 3.83-3.93 (m, 1H), 3.97 (s, 3H), 5.15-5.25 (m, 1H), 7.38 (s, 1H).

### Step 10: Preparation of tert-butyl (1-(3,6-dimethoxy-5-pentylpyridin-2-yl)butan-2-yl)carbamate

A mixture of tert-butyl N-[1-[(5-bromo-3,6-dimethoxy-2-pyridyl)methyl]propyl]carbamate (780 mg, 2.00 mmol, 1 eq), pentylboronic acid (348.54 mg, 3.01 mmol, 1.5 eq), cesium carbonate (1.96 g, 6.01 mmol, 3 eq) in toluene (10 mL) and water (1 mL) was degassed and purged with N₂ for 3 times at 20° C. for 10 min, and then Pd(dppf)Cl₂·CH₂Cl₂ (163.63 mg, 200.37 μmol, 0.1 eq) was added to the mixture and stirred at 90° C. for 13 h under N₂ atmosphere. On completion, the residue was diluted with water (2 mL) and extracted with ethyl acetate (3 mL×3). The combined organic layers were washed with brine (1 mL), dried over Na₂SO₄, filtered, and concentrated under reduced pressure to give a residue that was purified by column chromatography (SiO₂, petroleum ether/ethyl acetate gradient, 100:1 to 30:1) to afford tert-butyl (1-(3,6-dimethoxy-5-pentylpyridin-2-yl)butan-2-yl)carbamate (400 mg, 1.05 mmol, 52.46% yield) as a white solid. ¹H NMR (400 MHz, CHLOROFORM-d) δ ppm 0.88-0.98 (m, 6H), 1.29-1.38 (m, 4H), 1.40 (s, 9H), 1.43-1.52 (m, 2H), 1.53-1.61 (m, 2H), 2.46-2.58 (m, 2H), 2.76-2.86 (m, 1H), 2.87-2.96 (m, 1H), 3.77 (s, 3H), 3.81-3.88 (m, 1H), 3.90 (s, 3H), 5.84 (br d, J=6.58 Hz, 1H), 6.99 (s, 1H).

### Step 11: Preparation of 1-(3,6-dimethoxy-5-pentylpyridin-2-yl)butan-2-amine (1)

To a solution of tert-butyl (1-(3,6-dimethoxy-5-pentylpyridin-2-yl)butan-2-yl)carbamate (400 mg, 1.05 mmol, 1 eq) in TFA (1 mL) and DCM (4 mL) was added triethylsilane (366.69 mg, 3.15 mmol, 503.69 μL, 3 eq). The mixture was stirred at 20° C. for 1 h. On completion, the residue was purified by prep-HPLC (column: Phenomenex Luna C18 80×40 mm×3 μm; mobile phase: [water (0.1% HCl)−ACN]; B %: 5%-40%, 7 min). Compound 1 (100 mg, 356.63 μmol, 33.93% yield) was obtained as a white solid. ¹H NMR (400 MHz, METHANOL-d4) δ ppm 0.91 (t, J=6.97 Hz, 3H), 1.07 (t, J=7.52 Hz, 3H), 1.27-1.41 (m, 4H), 1.59 (dt, J=14.92, 7.46 Hz, 2H), 1.72 (quin, J=7.31 Hz, 2H), 2.52-2.61 (m, 2H), 2.92 (dd, J=15.71, 8.13 Hz, 1H), 3.11 (dd, J=15.65, 4.65 Hz, 1H), 3.55-3.67 (m, 1H) 3.83 (s, 3H) 3.90 (s, 3H) 7.27 (s, 1H); ¹³C NMR (101 MHz, METHANOL-d4) δ ppm 155.50, 148.80, 138.07, 124.30, 123.07, 55.54, 52.47, 52.33, 31.69, 31.36, 29.38, 28.78, 25.44, 22.13, 12.94, 8.66.

### Example 2: Preparation of 1-(3,6-dimethoxy-5-methylpyridin-2-yl)butan-2-amine (2) hydrochloride

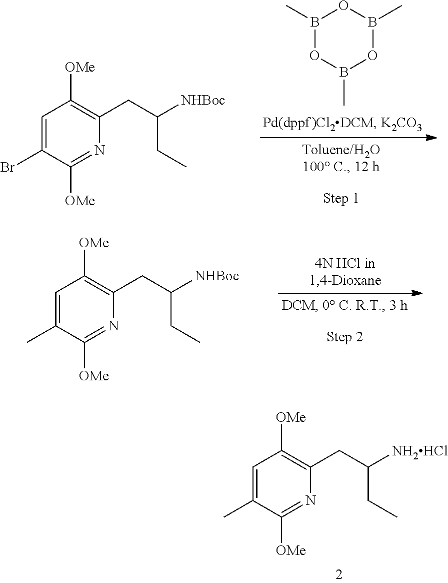

### Step 1: Preparation of tert-butyl (1-(3,6-dimethoxy-5-methylpyridin-2-yl)butan-2-yl)carbamate

To a stirred solution of tert-butyl (1-(5-bromo-3,6-dimethoxypyridin-2-yl)butan-2-yl)carbamate (0.100 g, 1 eq, 0.257 mmol) in toluene (4 mL) and water (1 mL) was added trimethyl-1,3,5,2,4,6-trioxatriborinane (80.6 mg, 1.2 eq, 0.321 mmol) and potassium carbonate (107 mg, 3 eq, 0.771 mmol). The reaction mixture was degassed with argon for 5 min, then PdCl₂(dppf)·DCM (21 mg, 0.1 eq, 0.0257 mmol) was added, and reaction mixture was heated at 100° C. for 12 h. The progress of reaction was monitored by TLC and LC-MS. After completion, the reaction mixture was diluted with water and extracted with ethyl acetate. The combined organic layers were washed with brine solution, dried over anhydrous Na₂SO₄, filtered, and solvent was evaporated under residue pressure to give a crude material which was purified by column chromatography (SiO₂, 10% Ethyl acetate/Heptane). The desired fraction was concentrated under reduced pressure to afford tert-butyl (1-(3,6-dimethoxy-5-methylpyridin-2-yl)butan-2-yl)carbamate (65 mg, 78% yield) as a colorless liquid. LC-MS m/z 325.10 [M+1]⁺; ¹H NMR (400 MHz, DMSO-d₆) δ=7.26 (s, 1H), 6.53 (d, J=8.8 Hz, 1H), 3.80 (s, 3H), 3.72 (s, 3H), 2.76-2.63 (m, 2H), 2.10 (s, 3H), 1.39-1.23 (m, 11H), 0.80 (t, J=7.6 Hz, 3H).

### Step 2: Preparation of 1-(3,6-dimethoxy-5-methylpyridin-2-yl)butan-2-amine (2) hydrochloride

To a stirred solution of tert-butyl (1-(3,6-dimethoxy-5-methylpyridin-2-yl)butan-2-yl)carbamate (60 mg, 0.185 μmol) in 1,4-dioxane (3 mL, 35.2 mmol) cooled to 0° C. was added 4 N HCl in 1,4-dioxane (2 mL). The reaction progress was monitored by TLC and LC-MS. After completion of reaction, the mixture was concentrated under reduced pressure to afford a crude material which was triturated with DCM (0.2 mL) and diethyl ether (2.0 mL) followed by n-pentane (5 mL). The resulting compound was dried under reduced pressure to afford Compound 2 HCl (Yield: 23 mg, 55.44%) as a semi-solid. LC-MS m/z 225.30 [M+1]⁺; ¹H NMR (400 MHz, DMSO-d₆) δ=7.82 (br s, 3H), 7.38 (s, 1H), 3.83 (s, 3H), 3.76 (s, 3H), 3.53-3.52 (m, 1H), 2.96-2.83 (m, 2H), 2.14 (s, 3H), 1.58-1.54 (m, 2H), 0.93 (t, J=7.6 Hz, 3H).

### Example 3: Preparation of 1-(5-bromo-3,6-dimethoxypyridin-2-yl)butan-2-amine (3) hydrochloride

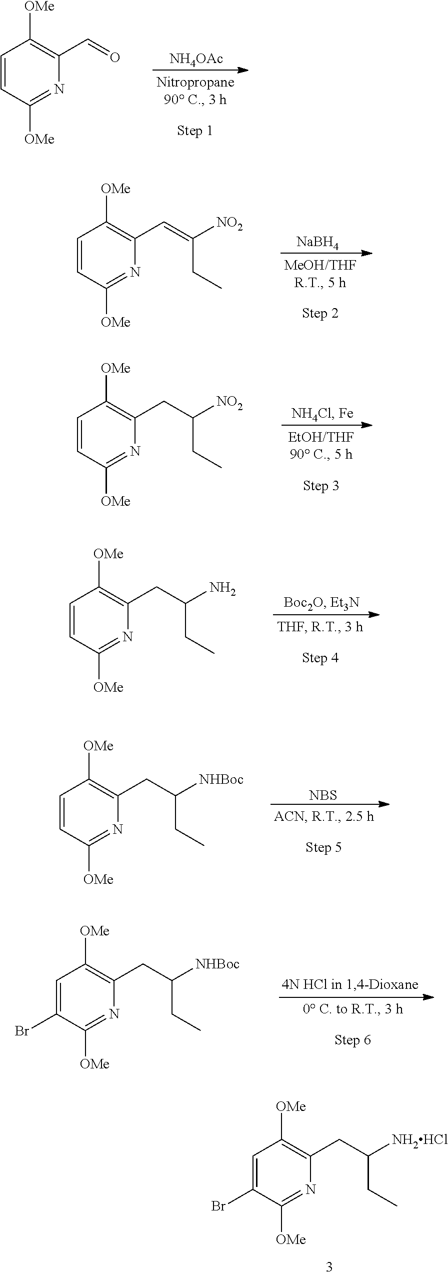

### Step 1: Preparation of (E)-3,6-dimethoxy-2-(2-nitrobut-1-en-1-yl)pyridine

To a stirred solution of 3,6-dimethoxypicolinaldehyde (1 g, 1 eq 6.05 mmol) in 1-nitropropane (40 mL) was added ammonium acetate (0.7 g, 1.5 eq, 9.08 mmol) portion-wise. After addition, the reaction mixture was allowed to stir at 90° C. for 3 h. The progress of the reaction was monitored by TLC and LC-MS. After completion, the reaction mixture was diluted with water extracted with ethyl acetate. The combined organic layers were washed with brine and dried over anhydrous Na₂SO₄, filtered, and solvent was evaporated under reduced pressure to afford a crude compound. The crude compound was purified by column chromatography (SiO₂, 10% Ethyl acetate/Heptane). The desired fraction was concentrated under reduced pressure to afford (E)-3,6-dimethoxy-2-(2-nitrobut-1-en-1-yl)pyridine (790 mg, 54.78% yield) as a yellow solid. LC-MS m/z 239.10 [M+1]⁺; ¹H NMR (400 MHz, CDCL₃) δ=8.294 (s, 1H), 7.29-7.26 (m, 1H), 6.80 (d, J=9.2 Hz, 1H), 3.90-3.77 (m, 6H), 3.39-3.34 (m, 2H), 1.28-1.23 (m, 3H).

### Step 2: Preparation of 3,6-dimethoxy-2-(2-nitrobutyl)pyridine

To a stirred solution of (E)-3,6-dimethoxy-2-(2-nitrobut-1-en-1-yl)pyridine (0.790 g, 1 eq, 3.32 mmol) in methanol (10 mL) and THF (5 mL) cooled to 0° C. was added NaBH₄ (752 mg, 8 eq, 19.90 mmol) portion-wise over 15 min. After addition, the reaction mixture was allowed to stir at room temperature for 5 h. The progress of reaction was monitored by TLC and LC-MS. On completion, the reaction mixture was diluted with NH₄Cl solution (20 ml) and extracted with ethyl acetate. The combined organic layers were washed with brine and dried over anhydrous Na₂SO₄, filtered, and concentrated under reduced pressure to afford 3,6-dimethoxy-2-(2-nitrobutyl)pyridine (crude compound) that was directly used for next step without further purification. LC-MS m/z 241.20 [M+1]+.

### Step 3: Preparation of 1-(3,6-dimethoxypyridin-2-yl)butan-2-amine

To a stirred solution of 3,6-dimethoxy-2-(2-nitrobutyl)pyridine (640 mg, 2.66 mmol, 1 eq) in ethanol (7 mL) and H₂O (3 mL) was added iron powder (1.79 g, 31.97 mmol, 12 eq) and NH₄Cl (854 mg, 15.98 mmol, 10 eq). The mixture was stirred at 90° C. for 5 h. On completion, the mixture was filtered and concentrated under reduced pressure to give an aqueous solution. The pH was adjusted to 9 by saturated sodium bicarbonate, and the suspension was filtered and dried to afford 1-(3,6-dimethoxypyridin-2-yl) butan-2-amine (Yield: 550 mg, 97.21%) as a brown solid (crude). The crude compound was directly used for the next step without further purification. LC-MS m/z 211.20 [M+1]+.

### Step 4: Preparation of tert-butyl (1-(3,6-dimethoxypyridin-2-yl)butan-2-yl)carbamate

To a solution of 1-(3,6-dimethoxypyridin-2-yl)butan-2-amine (550 mg, 2.62 mmol) in tetrahydrofuran (5 mL) cooled to 0° C. under inert atmosphere was added trimethylamine (1.1 mL, 3 eq, 7.85 mmol) and di-tert-butyl dicarbonate (0.9 mL, 1.5 eq, 3.92 mmol). The mixture was stirred at room temperature for 3 h. The progress of reaction was monitored by TLC and LC-MS. After completion, the reaction mixture was diluted with water extracted with ethyl acetate. The combined organic layers were washed with brine and dried over anhydrous Na₂SO₄, filtered, and solvent was evaporated under reduced pressure to afford a crude compound. The crude compound was purified by column chromatography (SiO₂, 10% Ethyl acetate/Heptane). The desired fraction was concentrated under reduced pressure to afford tert-butyl (1-(3,6-dimethoxypyridin-2-yl)butan-2-yl)carbamate (Yield: 510 mg, 56%) as a light-yellow solid. LC-MS m/z 311.10 [M+1]+.

### Step 5: Synthesis of tert-butyl (1-(5-bromo-3,6-dimethoxypyridin-2-yl)butan-2-yl)carbamate

To a stirred solution of tert-butyl (1-(3,6-dimethoxypyridin-2-yl) butan-2-yl)carbamate (510 mg, 1.64 mmol, 1 eq) in acetonitrile (10 mL) cooled to 0° C. was added NBS (439 mg, 2.46 mmol, 1.5 eq). The reaction mixture was allowed to stir at room temperature for 2.5 h. The reaction progress was monitored by TLC and LC-MS. After completion, the reaction mixture was diluted with water extracted with ethyl acetate. The combined organic layers were washed with brine and dried over anhydrous Na₂SO₄, filtered, and solvent was evaporated under reduced pressure to afford a crude compound. The crude compound was purified by column chromatography (SiO₂, 10% Ethyl acetate/Heptane). The desired fraction was concentrated under reduced pressure to afford tert-butyl (1-(5-bromo-3,6-dimethoxypyridin-2-yl)butan-2-yl)carbamate as an off-white solid (Yield: 460 mg, 72%). LC-MS m/z 390.90 [M+1]+; ¹H NMR (400 MHz, DMSO-d₆) δ=7.69 (s, 1H), 6.54 (d, J=9.2 Hz, 1H), 3.85 (s, 3H), 3.77 (s, 3H), 2.79-2.61 (m, 2H), 1.44-1.36 (m, 2H), 1.34 (s, 9H), 0.82 (t, J=7.2 Hz, 3H).

### Step 6: Preparation of 1-(5-bromo-3,6-dimethoxypyridin-2-yl)butan-2-amine (3) hydrochloride

To a stirred the solution of tert-butyl (1-(5-bromo-3,6-dimethoxypyridin-2-yl)butan-2-yl)carbamate (80 mg, 206 μmol) in 1,4-dioxane (3 mL), cooled to 0° C. was added 4 N HCl in 1,4-dioxane (3 mL). The reaction mixture was then allowed to stir at room temperature for 3 h. The progress of reaction was monitored by TLC and LC-MS. After completion of reaction, the mixture was concentrated under reduced pressure to get a crude material which was triturated with DCM (0.2 mL) and diethyl ether (2 mL) followed by n-pentane (5 mL). The resulting compound was dried under reduced pressure to afford Compound 3 HCl as a semi-solid (Yield: 48 mg, 71.7%). LC-MS m/z 289.05 [M+1]⁺; ¹H NMR (400 MHz, DMSO-d₆): δ=7.82 (s, 1H), 7.73 (br s, 2H), 3.88 (s, 3H), 3.80 (s, 3H), 3.56-3.53 (m, 1H), 2.94 (d, J=6 & 15.6 Hz, 1H), 2.84 (d, J=7.2 & 15.2 Hz, 111), 1.61-1.54 (m, 211), 0.94 (t, J=7.6 Hz, 3H).

### Example 4: Preparation of 1-(3,6-dimethoxy-5-pentylpyridin-2-yl)propan-2-amine (4) formate

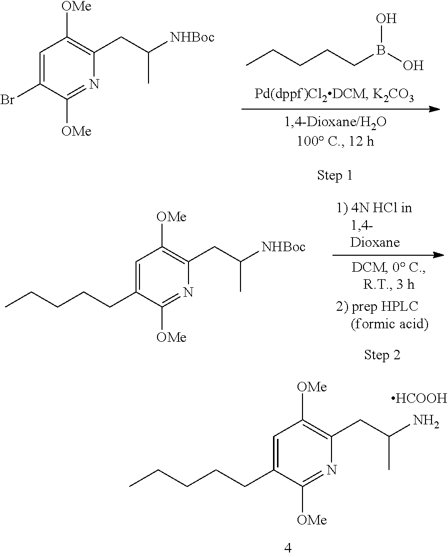

### Step 1: Preparation of tert-butyl (1-(3,6-dimethoxy-5-pentylpyridin-2-yl)propan-2-yl)carbamate

To mixture of tert-butyl N-[1-(5-bromo-3,6-dimethoxypyridin-2-yl)propan-2-yl]carbamate (0.3 g, 799 μmol), pentylboronic acid (232 mg, 2.5 eq, 2 mmol), potassium carbonate (110 mg, 3 eq, 799 μmol) in 1,4-dioxane (15 mL, 176 mmol) and water (3 mL, 167 mmol) was added PdCl₂(dppf)·DCM (64.8 mg, 0.1 eq, 79.9 μmol) under inert atmosphere and stirred at 100° C. for 12 h. The progress of reaction was monitored by TLC and LC-MS. After completion, the reaction mixture was diluted with water and organic layer was extracted with ethyl acetate. The combined organic layers were washed with brine and dried over anhydrous Na₂SO₄, filtered, and concentrated under reduced pressure to give a residue that was purified by silica gel column chomatography, using ethyl acetate and hexane as eluent. The desired fraction was concentrated under reduced pressure afford tert-butyl N-[1-(3,6-dimethoxy-5-pentylpyridin-2-yl)propan-2-yl]carbamate (44 mg, 191 μmol) as an off-white solid. LC-MS m/z 367 [M+1]⁺; ¹H NMR (400 MHz, DMSO-d₆) δ=7.23 (s, 1H), 6.64 (d, J=8 Hz, 1H), 3.96-3.93 (m, 1H), 3.80 (s, 3H) 3.74 (s, 3H), 2.70-2.66 (m, 3H), 1.55-1.47 (m, 2H), 1.31 (m 12H),1.29-1.28 (m, 2H), 0.98 (d, J=6.4 Hz, 3H)), 0.82 (d, J=11.6 Hz, 3H).

### Step 2: Preparation of 1-(3,6-dimethoxy-5-pentylpyridin-2-yl)propan-2-amine (4) formate

To a stirred solution of tert-butyl N-[1-(3,6-dimethoxy-5-pentylpyridin-2-yl)propan-2-yl]carbamate (44 mg, 1 eq) in 1,4-dioxane (3 mL, 35.2 mmol) under inert atmosphere was added 4 N HCl in 1,4-dioxane (43.8 mg, 5 eq) at 0° C. The reaction mixture was then allowed to stir for 3 h at room temperature. The progress of reaction was monitored by TLC and LC-MS. After completion of reaction, the mixture was concentrated under reduced pressure to provide a crude material which was triturated with DCM and diethyl ether, followed by n-pentane. This material was dried under reduced pressure to afford crude Compound 4 HCl as a brown, sticky solid. This was further purified by prep HPLC with formic acid to afford Compound 4 formate (Yield: 17 mg, 46.76%). LC-MS m/z 367[M+1]⁺; ¹H NMR (400 MHz, DMSO-d₆) δ 8.40 (br s, 1H), 7.29 (s, 1H), 3.81 (s, 3H), 3.75 (s, 3H) 3.74 (s, 3H), 3.54-3.52 (m, 1H), 2.50-2.47 (m, 2H), 1.56-1.53 (m 2H), 1.33-1.23 (m, 4H), 1.11 (d, J=6.4 Hz, 3H)), 0.86 (t, J=13.6 Hz, 3H).

### Example 5: Preparation of 1-(3,6-dimethoxy-5-methylpyridin-2-yl)propan-2-amine (5) hydrochloride

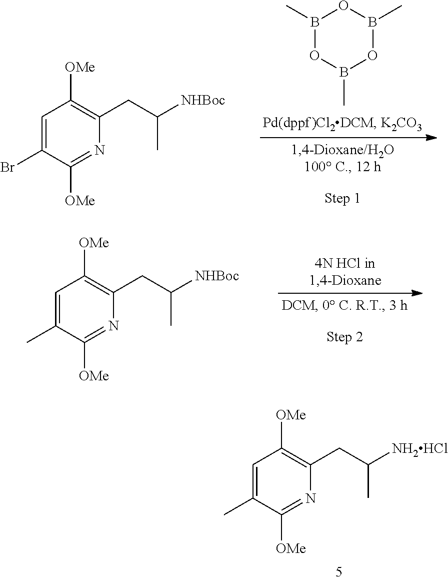

### Step 1: Preparation of tert-butyl (1-(3,6-dimethoxy-5-methylpyridin-2-yl)propan-2-yl)carbamate

To a stirred solution of tert-butyl (1-(5-bromo-3,6-dimethoxypyridin-2-yl)propan-2-yl)carbamate (0.050 g, 0.133 mmol, 1 eq) in 1,4-dioxane (2 mL) and water (1 mL) was added trimethyl-1,3,5,2,4,6-trioxatriborinane (46.6 μL, 1.5 eq, 0.167 mmol) and potassium carbonate (55.2 mg, 3 eq, 0.4 mmol). Reaction mixture was degassed with argon for 5 min, then PdCl₂(dppf)·DCM (10.9 mg, 0.1 eq, 0.0133 mmol) was added, and the mixture was heated at 100° C. for 12 h. The progress of reaction was monitored by TLC and LC-MS. After completion, the reaction mixture was diluted with water and extracted with ethyl acetate. The combined organic layers were washed with brine solution and dried over anhydrous Na₂SO₄, filtered and solvent was evaporated under reduced pressure to give a crude compound that was purified by column chomatography (SiO₂, 10% Ethyl acetate/Heptane). Desired fraction was concentrated under reduced pressure to afford tert-butyl (1-(3,6-dimethoxy-5-methylpyridin-2-yl)propan-2-yl) carbamate (Yield: 30 mg, 72.54% yield) as a colorless liquid. LC-MS m/z 311.10 [M+1]⁺; ¹H NMR (400 MHz, DMSO-d₆) δ=7.28 (s, 1H), 6.65 (d, J=8 Hz, 1H), 3.95-3.90 (m, 1H), 3.80 (s, 3H), 3.72 (s, 3H).

### Step 2: Preparation of 1-(3,6-dimethoxy-5-methylpyridin-2-yl)propan-2-amine (5) hydrochloride

To a stirred solution of tert-butyl (1-(3,6-dimethoxy-5-methylpyridin-2-yl)propan-2-yl) carbamate (60 mg, 193 mmol) in 1,4-dioxane (3 mL, 35.2 mmol) at 0° C. was added 4 N HCl in 1,4-dioxane (3 mL). The reaction mixture was then allowed to stir at room temperature for 3 h. The reaction progress was monitored by TLC and LC-MS. After completion of reaction, the mixture was concentrated under reduced pressure to afford a crude material which was triturated with DCM (0.2 mL) and diethyl ether (2.0 mL), followed by n-pentane (5 mL). The resulting compound was dried under reduced pressure to afford Compound 5 HCl as an off-white solid (Yield: 45 mg, 94%). LC-MS m/z 211.20 [M+1]⁺; ¹H NMR (400 MHz, DMSO-d₆) δ=7.82 (s, 3H), 7.38 (s, 1H), 3.80 (s, 3H), 3.75 (s, 3H), 3.63-3.57 (m, 1H), 2.93-2.82 (m, 2H), 2.14 (s, 3H), 1.18 (d, J=6.4 Hz, 3H).

### Example 6: Preparation of 1-(5-bromo-3,6-dimethoxypyridin-2-yl)propan-2-amine (6) hydrochloride

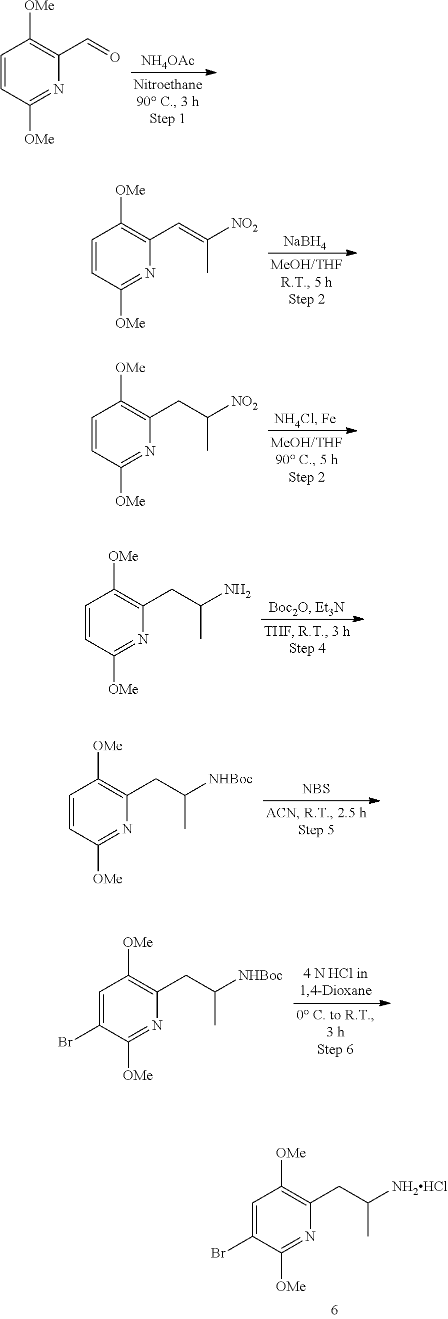

### Step 1: Preparation of (E)-3,6-dimethoxy-2-(2-nitroprop-1-en-1-yl)pyridine

To a stirred solution of 3,6-dimethoxypicolinaldehyde (4 g, 23.9 mmol) in nitroethane (40 mL was added ammonium acetate (2.77 g, 1.5 eq, 35.9 mmol) portion-wise. After addition, the reaction mixture was allowed to stir at 90° C. for 3 h. The reaction progress was monitored by TLC and LC-MS. After completion, the reaction mixture was diluted with water extracted with ethyl acetate. The combined organic layers were washed with brine and dried over anhydrous Na₂SO₄, filtered, and solvent was evaporated under reduced pressure to afford a crude compound. The crude compound was purified by column chromatography (SiO₂, 10% Ethyl acetate/Heptane). The desired fraction was concentrated under reduced pressure to afford (E)-3,6-dimethoxy-2-(2-nitroprop-1-en-1-yl)pyridine (Yield: 2.2 g, 41.01%) as a yellow solid. LC-MS m/z 225.20 [M+1]⁺; ¹H NMR (400 MHz, DMSO-d₆) δ=8.19 (s, 1H), 7.67 (d, J=9.2 Hz, 1H), 6.98 (d, J=8.8 Hz, 1H), 3.88 (s, 3H), 3.80 (s, 3H), 2.75 (s, 3H).

### Step 2: Preparation of 3,6-dimethoxy-2-(2-nitropropyl)pyridine

To a stirred solution of (E)-3,6-dimethoxy-2-(2-nitroprop-1-en-1-yl) pyridine (1.0 g, 1 eq, 4.46 mmol) in THE (15 mL, 184 mmol) and MeOH (30 mL, 494 mmol) was added NaBH₄ (1.34 g, 8 eq, 35.68 mmol) at 0° C. The reaction mixture was allowed to stir at room temperature for 5 h. The progress of reaction was monitored by TLC and LC-MS. On completion, the reaction mixture was diluted with NH₄Cl solution (20 ml) and extracted with ethyl acetate. The combined organic layers were washed with brine and dried over anhydrous Na₂SO₄, filtered and concentrated under reduced pressure to afford 3,6-dimethoxy-2-(2-nitropropyl) pyridine. The crude compound was directly used for the next step with further purification (Yield: 980 mg, 97%). LC-MS m/z 227.10 [M+1]+.

### Step 3: Preparation of 1-(3,6-dimethoxypyridin-2-yl)propan-2-amine

To a stirred solution of 3,6-dimethoxy-2-(2-nitropropyl)pyridine (980 mg, 4.33 mmol, 1 eq) in ethanol (15 mL) and H₂O (5 mL) was added iron powder (2.9 g, 52 mmol, 12 eq) and NH₄C₁ (2.32 g, 43.3 mmol, 10 eq). The mixture was stirred at 90° C. for 5 h. On completion, the mixture was filtered and concentrated under reduced pressure to give aqueous solution. The pH was adjusted to 9 by saturated sodium bicarbonate, and the suspension was filtered and dried under reduced pressure to afford 1-(3,6-dimethoxypyridin-2-yl) propan-2-amine (Yield: 800 mg, 94.1%) as a brown solid (crude) that was directly used for next step without further purification. LC-MS m/z 197.20 [M+1]⁺.

### Step 4: Preparation of tert-butyl (1-(3,6-dimethoxypyridin-2-yl)propan-2-yl)carbamate

To a stirred solution of 1-(3,6-dimethoxypyridin-2-yl)propan-2-amine (850 mg, 1 eq, 4.33 mmol) in THE (10 mL) cooled at 0° C. under inert atmosphere was added triethylamine (3.02 mL, 5 eq, 21.7 mmol) and di-tert-butyl dicarbonate (1.99 mL, 2 eq, 8.66 mmol). The reaction mixture was then allowed to stir at room temperature for 3 h. The reaction progress was monitored by TLC and LC-MS. After completion, the reaction mixture was diluted with water and extracted with ethyl acetate. The combined organic layers were washed with brine and dried over anhydrous Na₂SO₄, filtered, and solvent was evaporated under reduced pressure to afford a crude compound. The crude compound was purified by column chromatography (SiO₂, 10% Ethyl acetate/Heptane). The desired fraction was concentrated under reduced pressure to afford tert-butyl (1-(3,6-dimethoxypyridin-2-yl)propan-2-yl)carbamate (Yield: 0.790 g, 61.54%) as a pale-yellow solid. LC-MS m/z 269.10 [M+1]⁺; ¹H NMR (400 MHz, DMSO-d₆) δ=7.39 (d, J=8 Hz, 1H), 6.67 (d, J=8 Hz, 1H), 6.61 (d, J=8 Hz, 1H), 3.99-3.95 (m, 1H), 3.77 (s, 3H), 3.73 (s, 3H), 2.73 (d, J=8 Hz, 2H), 1.32 (s, 9H), 0.98 (d, J=8 Hz, 3H).

### Step 5: Preparation of tert-butyl (1-(5-bromo-3,6-dimethoxypyridin-2-yl)propan-2-yl)carbamate

To a stirred solution of tert-butyl (1-(3,6-dimethoxypyridin-2-yl)propan-2-yl) carbamate (0.6 g, 2.02 mmol, 1 eq) in acetonitrile (20 mL, 383 mmol) at 0° C. was added NBS (541 mg, 1.5 eq, 3.04 mmol). The reaction mixture was allowed to stir for 2.5 h at room temperature. The reaction progress was monitored by TLC and LC-MS. After completion, the reaction mixture was diluted with water extracted with ethyl acetate. The combined organic layers were washed with brine and dried over anhydrous Na₂SO₄, filtered, and solvent was evaporated under reduced pressure to afford a crude compound. The crude compound was purified by column chromatography (SiO₂, 10% Ethyl acetate/Heptane). The desired fraction was concentrated under reduced pressure to afford tert-butyl (1-(5-bromo-3,6-dimethoxypyridin-2-yl)propan-2-yl) carbamate (Yield: 610 mg, 80.29%). LC-MS m/z 376.85 [M+1]⁺; ¹H NMR (400 MHz, DMSO-d₆) δ=7.70 (s, 1H), 6.66 (d, J=8 Hz, 1H), 3.98 (br s, 1H), 3.96 (s, 3H), 3.94 (s, 3H), 2.72-2.66 (m, 211), 1.30 (s, 911), 1.00 (d, J=6.4 Hz, 311).

### Step 6: Preparation of 1-(5-bromo-3,6-dimethoxypyridin-2-yl)propan-2-amine (6) hydrochloride

To a stirred solution of tert-butyl (1-(5-bromo-3,6-dimethoxypyridin-2-yl)propan-2-yl)carbamate (120 mg, 0.320 mmol, 1 eq) in 1,4-dioxane (2 mL) cooled to 0° C. was added 4 N HCl in 1,4-dioxane (3 mL). The reaction mixture was then allowed to stir at room temperature for 3 h. The reaction progress was monitored by TLC and LC-MS. After completion of reaction, the mixture was concentrated under reduced pressure to afford a crude which was triturated with DCM (0.2 mL) and diethyl ether (2.0 mL), followed by n-pentane (5.0 mL). The resulting compound was dried under reduced pressure to afford Compound 6 HCl as an off-white solid (70 mg, 79.56% yield). LC-MS m/z 275.15 [M+1]⁺; ¹H NMR (400 MHz, DMSO-d₆) δ=6.89 (s, 1H), 3.13 (s, 3H), 3.05 (s, 3H) 3.03-2.99 (m, 1H), 2.20-2.14 (m, 2H), 0.53 (d, J=8 Hz, 3H).

### Example 7: Preparation of 2-(3,6-dimethoxy-5-pentylpyridin-2-yl)ethan-1-amine (7) hydrochloride

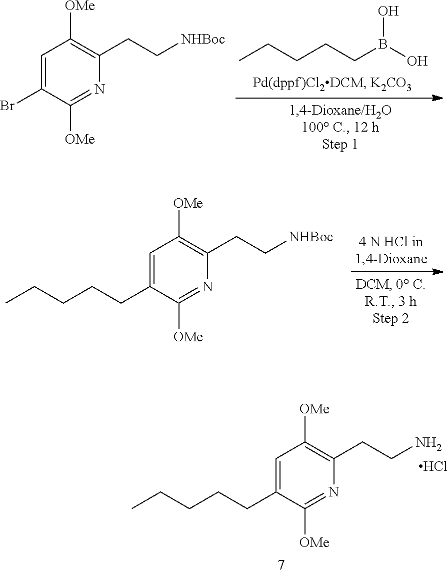

### Step 1: Preparation of tert-butyl (2-(3,6-dimethoxy-5-pentylpyridin-2-yl)ethyl)carbamate

To a stirred solution of tert-butyl (2-(5-bromo-3,6-dimethoxypyridin-2-yl)ethyl)carbamate (1.1 g, 1 eq, 3.05 mmol) in 1,4-dioxane (12 mL) and water (3 mL) was added pentylboronic acid (0.706 g, 2 eq, 6.09 mmol) and potassium carbonate (1.26 g, 3 eq, 9.14 mmol). The reaction mixture was degassed with argon for 5 min then PdCl₂(dppf)·DCM (247 mg, 0.1 eq, 0.305 mmol) was added, and resulting reaction mixture was heated at 100° C. for 12 h. The progress of reaction was monitored by TLC and LC-MS. After completion, the reaction mixture was diluted with water and extracted with ethyl acetate. The combined organic layers were washed with brine solution and dried over anhydrous Na₂SO₄, filtered and solvent was evaporated under residue pressure. This crude material was purified by column chromatography (SiO₂, 10% Ethyl acetate/Heptane), and desired fractions were concentrated under reduced pressure to afford tert-butyl (2-(3,6-dimethoxy-5-pentylpyridin-2-yl)ethyl)carbamate (Yield: 0.3 g, 27.95%) as a colorless liquid. LC-MS m/z 353.25 [M+1]+; ¹H NMR (400 MHz, DMSO-d₆) δ=7.23 (s, 1H), 6.79 (brs, 1H), 3.80 (s, 3H), 3.72 (s, 3H), 3.23-3.20 (m, 2H), 2.72 (t, J=7.6 Hz, 2H), 1.53-1.51 (m, 2H), 1.35 (s, 9H), 1.19-1.15 (m, 6H), 0.86 (t, J=8 Hz, 3H).

### Step 2: Preparation of 2-(3,6-dimethoxy-5-pentylpyridin-2-yl)ethan-1-amine (7) hydrochloride

To a stirred solution of tert-butyl (2-(3,6-dimethoxy-5-pentylpyridin-2-yl)ethyl)carbamate (35 mg, 1 eq, 0.0993 mmol) in 1,4-dioxane (3 mL) at 0° C. was added 4 N HCl in 1,4-dioxane (1 mL). The reaction progress was monitored by TLC and LC-MS. After completion of reaction, the mixture was concentrated under reduced pressure to afford a crude which was triturated with DCM (0.2 mL) and diethyl ether (2.0 mL) followed by n-pentane (5 mL). The resulting compound was dried under reduced pressure to afford Compound 7 HCl as an off-white solid (Yield: 23 mg, 91.78%). LC-MS m/z 253.30 [M+1]⁺; ¹H NMR (400 MHz, DMSO-d₆) δ=7.94 (bs, 3H), 7.31 (s, 1H), 3.82 (s, 3H), 3.76 (s, 3H), 3.18-3.13 (m, 2H), 2.93 (t, J=7.6 Hz, 2H), 2.50-2.46 (m, 2H), 1.54-1.50 (m, 2H), 1.31-1.25 (m, 4H), 0.86 (t, J=6.8 Hz, 3H).

### Example 8: Preparation of 2-(3,6-dimethoxy-5-methylpyridin-2-yl)ethan-1-amine (8) hydrochloride

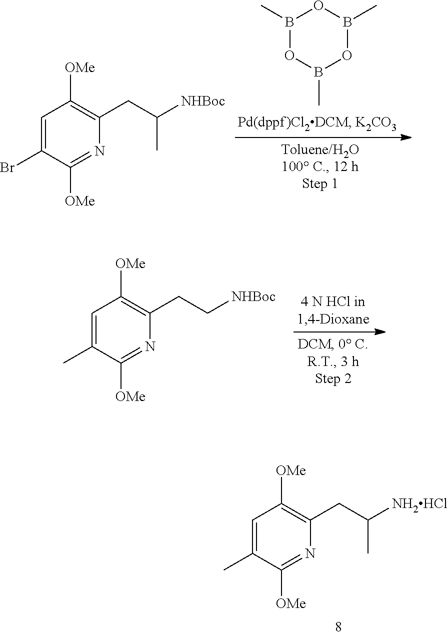

### Step 1: Preparation of tert-butyl (2-(3,6-dimethoxy-5-methylpyridin-2-yl)ethyl)carbamate

To a stirred solution of tert-butyl (2-(5-bromo-3,6-dimethoxypyridin-2-yl)ethyl)carbamate (0.100 g, 1 eq, 0.277 mmol) in toluene (4 mL) and water (1 mL) was added 2,4,6-trimethyl-1,3,5,2,4,6-trioxatriborinane (41.70 mg, 1.2 eq, 0.332 mmol) and potassium carbonate (115 mg, 3 eq, 0.830 mmol). The reaction mixture was degassed with argon for 5 min, PdCl₂(dppf)·DCM (22 mg, 0.1 eq, 0.027 mmol) was added, and the resulting mixture was heated at 100° C. for 12 h. The progress of reaction was monitored by TLC and LC-MS. After completion, the reaction mixture was diluted with water and extracted with ethyl acetate. The combined organic layers were washed with brine and dried over anhydrous Na₂SO₄, filtered, and solvent was evaporated under reduced pressure to give a crude material which was purified by column chromatography (SiO₂, 10% Ethyl acetate/Heptane). The desired fraction was concentrated under reduced pressure to afford tert-butyl (2-(3,6-dimethoxy-5-methylpyridin-2-yl)ethyl)carbamate as a colorless liquid (Yield: 60 mg, 73%). LC-MS m/z 297.05 [M+1]⁺; ¹H NMR (400 MHz, DMSO-d₆): δ=7.28 (s, 1H), 6.79 (s, 1H), 3.80 (s, 3H), 3.72 (s, 3H), 3.24-3.19 (m, 2H), 2.75 (m, 2H), 2.11 (s, 3H), 1.35 (s, 9H) ppm

### Step 2: Preparation of 2-(3,6-dimethoxy-5-methylpyridin-2-yl)ethan-1-amine (8) hydrochloride

To a stirred solution of tert-butyl (2-(3,6-dimethoxy-5-methylpyridin-2-yl)ethyl)carbamate (68 mg, 1 eq. 0.229 mmol) in 1,4-dioxane (3 mL) cooled to 0° C. was added 4 N HCl in 1,4-dioxane (1 mL). The reaction was allowed to stir for 3 h at room temperature. The reaction progress was monitored by TLC and LC-MS. After completion of reaction, the mixture was concentrated under reduced pressure to afford a crude material which was triturated with DCM (0.2 mL) and diethyl ether (2 mL) followed by n-pentane (5 mL). The resulting compound was dried under reduced pressure to afford Compound 8 HCl as an off-white solid (Yield: 42 mg, 93.27%). LC-MS m/z 197.20 [M+1]⁺; ¹H NMR (400 MHz, DMSO-d₆) δ=7.86 (bs, 3H), 7.36 (s, 1H), 3.83 (s, 3H), 3.76 (s, 3H), 3.18-3.13 (m, 211), 2.93 (t, J=7.6, 2H), 2.13 (s, 311).

### Example 9: Preparation of 2-(5-bromo-3,6-dimethoxypyridin-2-yl)ethan-1-amine (9) hydrochloride

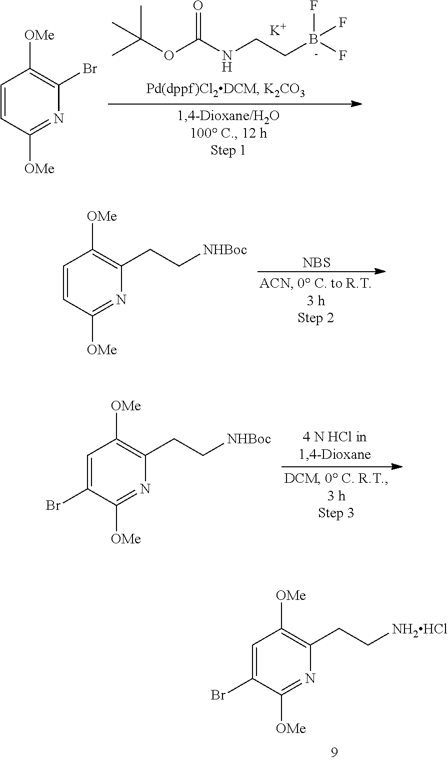

### Step 1: Preparation of tert-butyl (2-(3,6-dimethoxypyridin-2-yl)ethyl)carbamate

To a stirred solution 2-bromo-3,6-dimethoxypyridine of (5 g, 1 eq, 22.9 mmol) in 1,4-dioxane (100 mL) and water (30 mL) was added (2-((tert-butoxycarbonyl)amino)ethyl)trifluoroborate (10.4 g, 1.8 eq, 41.3 mmol) and potassium carbonate (9.51 g, 3 eq, 68.8 mmol). The reaction mixture was degassed with argon for 15 min, then Pd(dppf)Cl₂.DCM (1.87 g, 0.1 eq, 2.29 mmol) was added, and resulting mixture was heated at 100° C. for 12 h. The reaction progress was monitored by TLC and LC-MS. After completion, the reaction mixture was diluted with water and extracted with ethyl acetate. The combined organic layers were washed with brine and dried over anhydrous Na₂SO₄, filtered and solvent was evaporated under reduced pressure to afford a crude material which was purified by column chromatography (SiO₂, 10% Ethyl acetate/Heptane). The desired fraction was concentrated under reduced pressure to afford tert-butyl (2-(3,6-dimethoxypyridin-2-yl)ethyl)carbamate (Yield: 1.60 g, 24.71%) as a pale yellow oil. LC-MS m/z 283.15 [M+1]⁺; ¹H NMR (400 MHz, DMSO-d₆): δ=7.40 (d, J=8.8 Hz, 1H), 6.77 (bs, 1H), 6.62 (d, J=8.8 Hz, 1H), 3.79-3.75 (m, 6H), 3.30-3.25 (m, 2H), 2.78 (t, J=7.6 Hz, 2H), 1.36 (s, 9H).

### Step 2: Preparation of tert-butyl (2-(5-bromo-3,6-dimethoxypyridin-2-yl)ethyl)carbamate

To a stirred solution of tert-butyl (2-(3,6-dimethoxypyridin-2-yl)ethyl)carbamate (1.0 g, 3.54 mmol, 1 eq) in acetonitrile (20 mL) at 0° C. was added NBS (0.946 g, 1.5 eq, 5.31 mmol). The reaction mixture was allowed to stir for 3 h at room temperature. The reaction progress was monitored by TLC and LC-MS. After completion, the reaction mixture was diluted with water extracted with ethyl acetate. The combined organic layers were washed with brine, dried over anhydrous Na₂SO₄, filtered, and solvent was evaporated under reduced pressure to afford a crude material that was purified by column chromatography (SiO₂, 10% Ethyl acetate/Heptane). The desired fraction was concentrated under reduced pressure to afford tert-butyl (2-(5-bromo-3,6-dimethoxypyridin-2-yl)ethyl)carbamate (Yield: 1.1 g, 85.98%). LC-MS m/z 362.95[M+1]+; ¹H NMR (400 MHz, DMSO-d₆) δ=7.71 (s, 1H), 6.87 (s, 1H), 4.03 (s, 3H), 3.77 (s, 1H), 3.27-3.22 (m, 2H), 2.74 (t, J=7.2 Hz, 2H), 1.34 (s, 9H).

### Step 3: Preparation of 2-(5-bromo-3,6-dimethoxypyridin-2-yl)ethan-1-amine (9) hydrochloride

To a stirred solution of tert-butyl (2-(5-bromo-3,6-dimethoxypyridin-2-yl)ethyl)carbamate (60 mg, 1 eq) in 1,4-dioxane (2 mL) cooled to 0° C. was added 4 N HCl in 1,4-dioxane (5 eq) at 0° C. The reaction mixture was then allowed to stir at room temperature for 3 h. The reaction progress was monitored by TLC and LC-MS. After completion of reaction, the mixture was concentrated under reduced pressure to afford a crude material which was triturated with DCM (0.2 mL) and diethyl ether (2.0 mL) followed by n-pentane (5.0 mL). The resulting compound was dried under reduced pressure to afford Compound 9 HCl as an off-white solid (Yield: 38 mg, 87%). LC-MS m/z 263.0 [M+1]⁺; ¹H NMR (400 MHz, DMSO-d₆) δ=7.71 (s, 1H), 3.84 (s, 3H), 3.75 (s, 3H), 3.19 (t, J=7.2 Hz, 2H), 2.91 (t, J=6.8 Hz, 2H).

### Example 10: Preparation of 1-(2,5-dimethoxy-4-pentylphenyl)butan-2-amine (10) hydrochloride

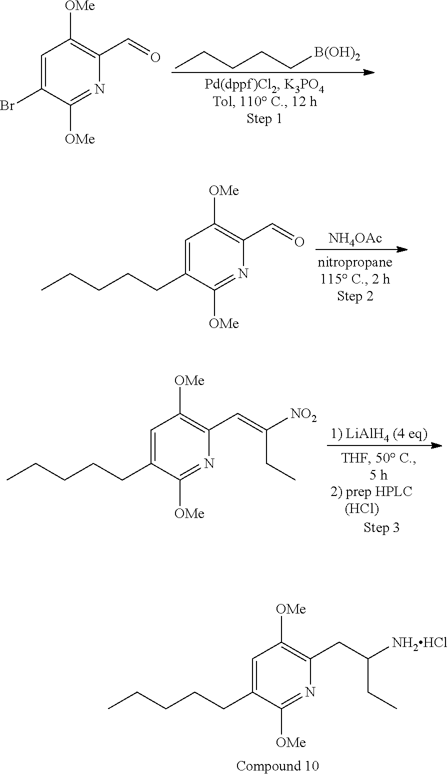

### Step 1: Preparation of 2,5-dimethoxy-4-pentylbenzaldehyde

The mixture solution of 4-bromo-2,5-dimethoxybenzaldehyde (6 g, 24.48 mmol, 1 eq), pentylboronic acid (4.26 g, 36.72 mmol, 1.5 eq), Pd(dppf)Cl₂ (895.71 mg, 1.22 mmol, 0.05 eq), and K₃PO₄ (15.59 g, 73.45 mmol, 3 eq) in toluene (100 mL) was stirred at 110° C. for 12 h. On completion, the mixture was filtered and concentrated. The residue was purified by column chromatography (SiO₂, petroleum ether/ethyl acetate gradient, 100:1 to 50:1) to afford 2,5-dimethoxy-4-pentylbenzaldehyde (5.1 g, 21.58 mmol, 88.15% yield) as a yellow oil.

### Step 2: Preparation of (E)-1,4-dimethoxy-2-(2-nitrobut-1-en-1-yl)-5-pentylbenzene

A mixture of 2,5-dimethoxy-4-pentylbenzaldehyde (1 g, 4.23 mmol, 1 eq) and NH₄OAc (652.37 mg, 8.46 mmol, 2 eq) in 1-nitropropane (6.79 g, 76.17 mmol, 6.80 mL, 18 eq) was stirred and warmed to 115° C. for 5 h. Upon completion, the solvent was removed. The residue was purified by column chromatography (SiO₂, petroleum ether/ethyl acetate gradient, 80:1 to 60:1) to afford (E)-1,4-dimethoxy-2-(2-nitrobut-1-en-1-yl)-5-pentylbenzene (830 mg, 2.70 mmol, 64% yield) as a yellow oil. ¹H NMR (400 MHz, CHLOROFORM-d) δ ppm 8.26 (s, 1H), 6.79 (s, 1H), 6.757 (s, 1H), 3.83-3.85 (m, 3H), 3.79-3.81 (m, 3H), 2.869 (d, J=7.2 Hz, 2H), 2.60-2.66 (m, 2H), 1.58-1.62 (m, 2H), 1.34-1.38 (m, 4H), 1.30 (t, J=7.2 Hz, 3H), 0.92 (t, J=6.8 Hz, 4H).

### Step 3: Preparation of 1-(2,5-dimethoxy-4-pentylphenyl)butan-2-amine (10) hydrochloride

A solution of (E)-1,4-dimethoxy-2-(2-nitrobut-1-en-1-yl)-5-pentylbenzene (830 mg, 2.70 mmol, 1 eq) in THF (20 mL) was cooled to 0° C. Then LiAlH₄ (409.89 mg, 10.80 mmol, 4 eq) was added. The mixture was stirred at 60° C. for 5 h. Upon completion, the mixture was cooled to 0° C. Then water (0.6 mL) was added. Then 30% aq. NaOH (0.6 mL) was added. The mixture was stirred to a smooth dispersion, then filtered and concentrated. The residue was purified prep-HPLC (column: Phenomenex Luna C18 80×40 mm×3 μm; mobile phase: [water (0.04% HCl)−ACN]; B %: 22%-50%, 7 min) to afford Compound 10 HCl (350 mg, 1.07 mmol, 40% yield, 96.8% purity, HCl) as a white solid. LC-MS(R_{T}=2.320 min, MS cal.: 279.42, [M+H]⁺ found=280.2); ¹H NMR (400 MHz, DMSO-d₆) δ ppm 8.06-7.90 (m, 3H), 6.82 (s, 1H), 6.78 (s, 1H), 3.77-3.69 (m, 6H), 3.28-3.20 (m, 1H), 2.80 (d, J=6.4 Hz, 2H), 2.54-2.51 (m, 2H), 1.55-1.46 (m, 4H), 1.35-1.25 (m, 4H), 0.89 (td, J=7.2, 18.4 Hz, 6H); ¹³C NMR (101 MHz, DMSO-d₆) δ ppm 151.00, 150.71, 129.77, 122.17, 114.06, 112.99, 55.84, 55.81, 52.19, 32.58, 31.16, 29.57, 29.22, 24.73, 21.91, 13.87, 9.40.

### Example 11: Preparation of 1-(2,5-dimethoxy-6-pentylpyridin-3-yl)butan-2-amine (11) hydrochloride

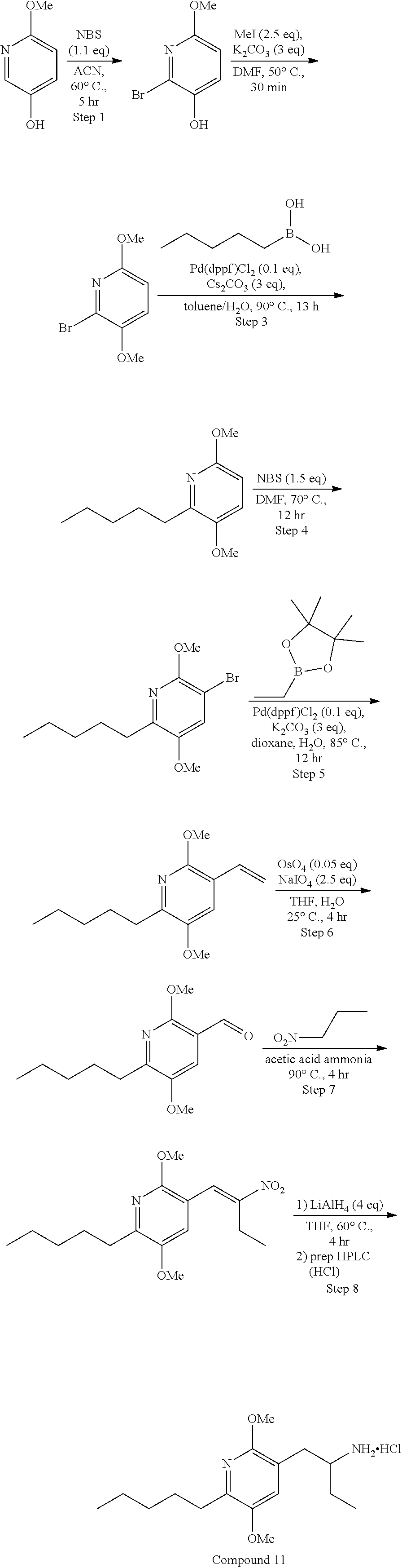

### Step 1: Preparation of 2-bromo-6-methoxypyridin-3-ol

To a solution of 6-methoxypyridin-3-ol (9.6 g, 76.72 mmol, 1 eq) in acetonitrile (50 mL) and water (7 mL), was added NBS (15.02 g, 84.40 mmol, 1.1 eq). The mixture was stirred at 60° C. for 5 h. On completion, the residue was diluted with water (30 mL) and extracted with ethyl acetate (20 mL×3). The combined organic layers were washed with brine (20 mL), dried over Na₂SO₄, filtered, and concentrated under reduced pressure to give a residue that was purified by column chromatography (SiO₂, petroleum ether/ethyl acetate gradient, 50:1 to 30:1). 2-bromo-6-methoxypyridin-3-ol (7 g, 34.31 mmol, 44.72% yield) was obtained as a white solid. ¹H NMR (400 MHz, CHLOROFORM-d) δ ppm 3.89 (s, 3H), 5.04 (br s, 1H), 6.66 (d, J=8.66 Hz, 1H), 7.26 (s, 1H), 7.28 (s, 1H).

### Step 2: Preparation of 2-bromo-3,6-dimethoxypyridine

To a solution of 2-bromo-6-methoxypyridin-3-ol (7 g, 34.31 mmol, 1 eq) in DMF (70 mL) was added K₂CO₃ (14.23 g, 102.93 mmol, 3 eq) and CH₃I (12.17 g, 85.78 mmol, 5.34 mL, 2.5 eq). The mixture was stirred at 50° C. for 0.5 h. On completion, the mixture was diluted with water (100 mL) and extracted with ethyl acetate (100 mL×3). The combined organic layers were washed with brine (80 mL), dried over Na₂SO₄, filtered, and concentrated under reduced pressure to give 2-bromo-3,6-dimethoxypyridine (6.7 g, 30.73 mmol, 89.56% yield) as a white solid. ¹H NMR (400 MHz, CHLOROFORM-d) δ ppm 3.87 (d, J=15.88 Hz, 6H), 6.67 (d, J=8.63 Hz, 1H), 7.20 (d, J=8.76 Hz, 1H).

### Step 3: Preparation of 3,6-dimethoxy-2-pentylpyridine

To a solution of 2-bromo-3,6-dimethoxypyridine (6.7 g, 30.73 mmol, 1 eq) in toluene (100 mL) and water (10 mL) was added pentylboronic acid (5.35 g, 46.09 mmol, 1.5 eq), Pd(dppf)Cl₂·CH₂Cl₂ (2.51 g, 3.07 mmol, 0.1 eq), and cesium carbonate (30.03 g, 92.18 mmol, 3 eq) under N₂ atmosphere. The mixture was stirred at 90° C. for 2 h. On completion, the mixture was extracted with ethyl acetate (100 mL×3). The combined organic layers were washed with brine (80 mL), dried over Na₂SO₄, filtered, and concentrated under reduced pressure to give a residue. The residue was purified by column chromatography (SiO₂, petroleum ether/ethyl acetate gradient, 50:1 to 3:1) to provide 3,6-dimethoxy-2-pentylpyridine (4.2 g, 20.07 mmol, 65.31% yield) as a yellow oil. ¹H NMR (400 MHz, CHLOROFORM-d) δ ppm 0.86-0.95 (m, 3H), 1.30-1.42 (m, 4H), 1.65-1.77 (m, 2H), 2.64-2.77 (m, 2H), 3.78 (s, 3H), 3.89 (s, 3H), 6.52 (d, J=8.75 Hz, 1H), 7.14 (d, J=8.75 Hz, 1H).

### Step 4: Preparation of 3-bromo-2,5-dimethoxy-6-pentylpyridine

To a solution of 3,6-dimethoxy-2-pentylpyridine (3.8 g, 18.16 mmol, 1 eq) in DMF (30 mL) was added NBS (4.85 g, 27.24 mmol, 1.5 eq). The mixture was stirred at 70° C. for 12 h. On completion, the mixture was poured into water (30 mL) and extracted with ethyl acetate (30 mL×3). The combined organic layers were washed with brine (20 mL), dried over Na₂SO₄, filtered, and concentrated under reduced pressure to give a residue that was purified by column chromatography (SiO₂, petroleum ether/ethyl acetate gradient, 1:0 to 30:1) to provide 3-bromo-2,5-dimethoxy-6-pentylpyridine (3 g, 10.41 mmol, 57.33% yield) as a yellow oil. ¹H NMR (400 MHz, CHLOROFORM-d) δ ppm 0.90 (t, J=6.94 Hz, 3H), 1.28-1.43 (m, 4H), 1.68 (quin, J=7.41 Hz, 2H), 2.63-2.71 (m, 2H), 3.78 (s, 3H), 3.96 (s, 3H), 7.34 (s, 1H).

### Step 5: Preparation of 2,5-dimethoxy-6-pentyl-3-vinylpyridine

To a solution of 3-bromo-2,5-dimethoxy-6-pentylpyridine (1 g, 3.47 mmol, 1 eq) in water (1 mL) and dioxane (10 mL), was added vinylboronic acid pinacol ester (1.60 g, 10.41 mmol, 1.77 mL, 3 eq) and K₂CO₃ (1.44 g, 10.41 mmol, 3 eq) at 20° C. Then Pd(dppf)Cl₂ (253.91 mg, 347.01 μmol, 0.1 eq) was added under N₂ atmosphere and the mixture was stirred at 85° C. for 12 h. On completion, the mixture was diluted with water (5 mL) and extracted with ethyl acetate (8 mL×3). The combined organic layers were washed with brine (3 mL), dried over Na₂SO₄, filtered, and concentrated under reduced pressure to give a residue that was purified by column chromatography (SiO₂, petroleum ether/ethyl acetate gradient, 1:0 to 50:1) to provide 2,5-dimethoxy-6-pentyl-3-vinylpyridine (0.2 g, 849.90 μmol, 24.49% yield) as a yellow oil.

### Step 6: Preparation of 2,5-dimethoxy-6-pentylnicotinaldehyde

To a solution of 2,5-dimethoxy-6-pentyl-3-vinylpyridine (0.3 g, 1.27 mmol, 1 eq) in THF (10 mL) and water (2.5 mL) was added OsO₄ (16.21 mg, 63.74 μmol, 3.31 μL, 0.05 eq) at 0° C. and the mixture was stirred for 10 min. Then NaTO₄ (681.70 mg, 3.19 mmol, 176.61 μL, 2.5 eq) and water (5 mL) were added, and the mixture was stirred at 25° C. for 4 h. On completion, water (10 mL) was added, and the mixture was extracted with ethyl acetate (8 mL×3). The combined organic layers were washed with brine (3 mL), dried over Na₂SO₄, filtered, and concentrated under reduced pressure to give a residue that was purified by prep-TLC (SiO₂, petroleum ether/ethyl acetate=10:1) to provide 2,5-dimethoxy-6-pentylnicotinaldehyde (0.3 g, 1.26 mmol, 99.17% yield) as a yellow oil. ¹H NMR (400 MHz, CHLOROFORM-d) δ ppm 0.89-0.94 (m, 3H), 1.33-1.40 (m, 4H), 1.68-1.76 (m, 2H), 2.74-2.83 (m, 2H), 3.81-3.86 (m, 3H), 3.95-4.05 (m, 3H), 7.55 (s, 1H), 10.32 (s, 1H).

### Step 7: Preparation of (E)-2,5-dimethoxy-3-(2-nitrobut-1-en-1-yl)-6-pentylpyridine

To a solution of 2,5-dimethoxy-6-pentylnicotinaldehyde (0.25 g, 1.05 mmol, 1 eq) in 1-nitropropane (3 mL) was added ammonium acetate (81.21 mg, 1.05 mmol, 1 eq). The mixture was stirred at 90° C. for 4 h. On completion, the mixture was extracted with ethyl acetate (3 mL×3). The combined organic layers were washed with brine (2 mL), dried over Na₂SO₄, filtered, and concentrated under reduced pressure to give a residue that was purified by prep-TLC (SiO₂, petroleum ether/ethyl acetate=10:1) to provide (E)-2,5-dimethoxy-3-(2-nitrobut-1-en-1-yl)-6-pentylpyridine (0.12 g, 389.14 μmol, 36.94% yield) as a yellow oil. ¹H NMR (400 MHz, CHLOROFORM-d) δ ppm 0.89-0.96 (m, 3H), 1.29 (t, J=7.40 Hz, 2H), 1.33-1.43 (m, 4H), 1.66-1.78 (m, 2H), 2.70-2.87 (m, 3H), 3.81 (s, 3H) 3.91-4.03, (m, 3H) 7.12 (s, 1H) 8.13 (s, 1H).

### Step 8: Preparation of 1-(2,5-dimethoxy-6-pentylpyridin-3-yl)butan-2-amine (11) hydrochloride

To a solution of (E)-2,5-dimethoxy-3-(2-nitrobut-1-en-1-yl)-6-pentylpyridine (0.1 g, 324.28 μmol, 1 eq) in THE (10 mL) was added LiAlH₄ (49.23 mg, 1.30 mmol, 4 eq) at 0° C., and the mixture was stirred at 60° C. for 3 h. On completion, the reaction mixture was quenched by addition of water (5 mL) and 30% of aq. NaOH (5 mL) at 0° C. The obtained suspension was filtered, and the filter cake was washed with THF (20 mL×3). The filtrate was concentrated to give a residue that was purified by prep-HPLC (column: Waters Xbridge BEH C18 100×30 mm×10 μm; mobile phase: [water(HCl)−ACN]; B %: 1%-25%, 8 min) to afford Compound 11 HCl (0.032 g, 114.14 μmol, 35.2% yield, HCl) as a white solid. ¹H NMR (400 MHz, METHANOL-d4) δ ppm 0.88-0.94 (m, 3H), 1.06 (t, J=7.50 Hz, 3H), 1.28-1.42 (m, 5H), 1.68 (m, 5H), 2.68-2.77 (m, 2H), 2.82-2.91 (m, 1H), 2.94-3.02 (m, 1H), 3.39-3.50 (m, 1H), 3.84 (s, 3H), 3.96 (s, 3H), 7.39 (s, 1H); ¹³C NMR (101 MHz, METHANOL-d4) δ ppm 154.97, 148.72, 146.64, 125.03, 116.02, 55.84, 52.88, 48.29, 47.86, 47.65, 47.43, 47.22, 47.01, 31.45, 30.47, 27.65, 25.35, 22.22, 13.04, 8.60.

### Example 12: Preparation of 1-(3,6-dimethoxy-5-pentylpyrazin-2-yl)butan-2-amine (12) hydrochloride

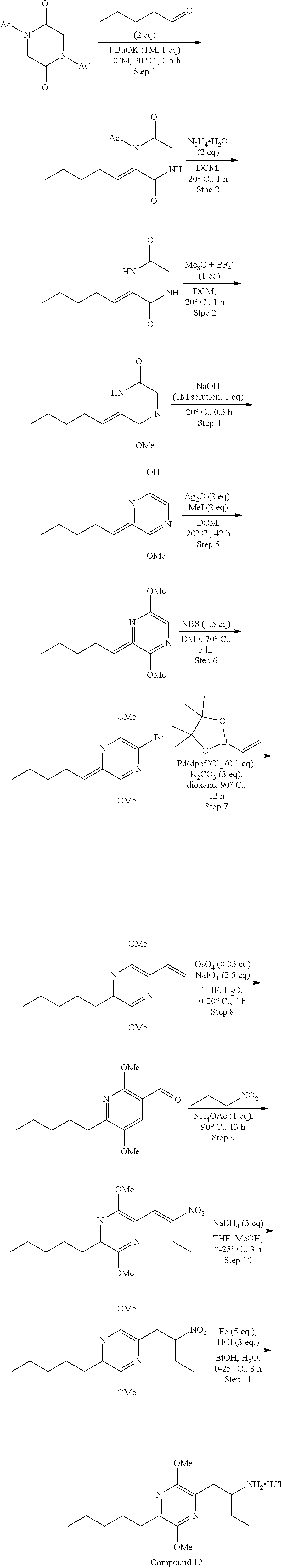

### Step 1: Preparation of (Z)-1-acetyl-6-pentylidenepiperazine-2,5-dione

To a solution of 1,4-diacetylpiperazine-2,5-dione (15 g, 75.69 mmol, 1 eq) in DCM (90 mL) was added t-BuOK (1 M in THF, 75.69 mL, 1 eq) and pentanal (13.04 g, 151.38 mmol, 16.10 mL, 2 eq) under N₂ atmosphere. The mixture was stirred at 20° C. for 0.5 h under N₂ atmosphere. On completion, the reaction mixture was filtered. The filtrate was poured into sat. aq. NH₄C₁ (50 mL) and extracted with DCM (50 mL×3). The combined organic layers were washed with brine (50 mL), dried over Na₂SO₄, filtered, and the filtrate was then concentrated. The crude product was purified by recrystallization from petroleum ether (100 mL) to afford (Z)-1-acetyl-6-pentylidenepiperazine-2,5-dione (13 g, 48.82 mmol, 64.50% yield) as a white solid. ¹H NMR (400 MHz, CHLOROFORM-d) δ ppm 8.93 (br s, 1H), 6.34 (t, J=7.9 Hz, 1H), 4.42 (s, 2H), 2.61 (s, 3H), 2.23 (q, J=7.6 Hz, 2H), 1.52-1.40 (m, 4H), 0.96-0.93 (m, 3H).

### Step 2: Preparation of (Z)-3-pentylidenepiperazine-2,5-dione

To a solution of (Z)-1-acetyl-6-pentylidenepiperazine-2,5-dione (14.6 g, 54.83 mmol, 1 eq) in DMF (150 mL) was added hydrazine hydrate (5.54 g, 109.65 mmol, 5.38 mL, 99% purity, 2 eq). The mixture was stirred at 20° C. for 1 h. On completion, the reaction mixture was quenched by addition of water (100 mL) and extracted with DCM (100 mL×3). The combined organic layers were washed with brine (100 mL) and dried over Na₂SO₄. The mixture was filtered and concentrated in vacuo to afford (Z)-3-pentylidenepiperazine-2,5-dione (8 g, 43.90 mmol, 80.08% yield) as a white solid. ¹H NMR (400 MHz, CHLOROFORM-d) δ ppm 9.92 (br s, 1H), 8.05 (br s, 1H), 5.72 (t, J=7.9 Hz, 1H), 3.91 (d, J=2.0 Hz, 2H), 2.16 (q, J=7.4 Hz, 2H), 1.38-1.25 (m, 4H), 0.90-0.84 (m, 3H).

### Step 3: Preparation of (Z)-5-methoxy-6-pentylidene-3,6-dihydropyrazin-2(1H)-one

To a solution of (Z)-3-pentylidenepiperazine-2,5-dione (8.5 g, 46.65 mmol, 1 eq) in DCM (240 mL) and CH₃NO₂ (64 mL) was added Me₃OBF₄ (6.90 g, 46.65 mmol, 1 eq). The mixture was stirred at 20° C. for 50 h. On completion, the reaction mixture was quenched by addition of water (500 mL) and extracted with DCM (50 mL×3). The combined organic layers were washed with brine (50 mL) and dried over Na₂SO₄. The mixture was filtered and concentrated in vacuo to afford (Z)-5-methoxy-6-pentylidene-3,6-dihydropyrazin-2(1H)-one (5 g, 25.48 mmol, 54.62% yield) as a white solid. ¹H NMR (400 MHz, CHLOROFORM-d) δ ppm 7.83 (br s, 1H), 5.59 (t, J=7.7 Hz, 1H), 4.33 (s, 2H), 3.76 (s, 3H), 2.08 (q, J=7.4 Hz, 2H), 1.49-1.32 (m, 4H), 0.95-0.90 (m, 3H).

### Step 4: Preparation of 5-methoxy-6-pentylpyrazin-2-ol

A solution of (Z)-5-methoxy-6-pentylidene-3,6-dihydropyrazin-2(1H)-one (5 g, 25.48 mmol, 1 eq) in aq. NaOH (1 M, 25.48 mL, 1 eq) was stirred at 20° C. for 0.5 h. On completion, the mixture was quenched with 1M aq. HCl (30 mL). The mixture was filtered and concentrated in vacuo to afford 5-methoxy-6-pentylpyrazin-2-ol (3.2 g, 16.31 mmol, 64.00% yield) as a white solid. ¹H NMR (400 MHz, CHLOROFORM-d) δ ppm 7.66 (s, 1H), 3.92 (s, 3H), 2.75-2.65 (m, 2H), 1.70-1.58 (m, 2H), 1.41-1.28 (m, 5H), 0.95-0.88 (m, 3H).

### Step 5: Preparation of 2,5-dimethoxy-3-pentylpyrazine

To a solution of 5-methoxy-6-pentylpyrazin-2-ol (3.2 g, 16.31 mmol, 1 eq) in DCM (100 mL) was added Ag₂O (7.56 g, 32.61 mmol, 2 eq) and CH₃I (4.63 g, 32.61 mmol, 2.03 mL, 2 eq). The mixture was stirred at 20° C. for 12 h. On completion, the mixture was filtered and concentrated in vacuo. The residue was purified by column chromatography (SiO₂, petroleum ether/ethyl acetate gradient, 50:1 to 1:1) to afford 2,5-dimethoxy-3-pentylpyrazine (3.2 g, 15.22 mmol, 93.33% yield) as a yellow oil.

### Step 6: Preparation of 2-bromo-3,6-dimethoxy-5-pentylpyrazine

To a solution of 2,5-dimethoxy-3-pentylpyrazine (3.2 g, 15.22 mmol, 1 eq) in DMF (32 mL) was added NBS (4.06 g, 22.83 mmol, 1.5 eq). The mixture was stirred at 70° C. for 5 h. On completion, the mixture was diluted with water (30 mL) and extracted with ethyl acetate (30 mL×3). The combined organic layers were washed with brine (20 mL), dried over Na₂SO₄, filtered, and concentrated under reduced pressure to give a residue. The residue was purified by column chromatography (SiO₂, petroleum ether/ethyl acetate, 50:1 to 1:1) to afford 2-bromo-3,6-dimethoxy-5-pentylpyrazine (2.5 g, 8.65 mmol, 56.81% yield) as a yellow oil. ¹H NMR (400 MHz, CHLOROFORM-d) δ ppm 3.98 (s, 3H), 3.93 (s, 3H), 2.71-2.66 (m, 2H), 1.69 (m, 2H), 1.42-1.30 (m, 4H), 0.96-0.86 (m, 3H).

### Step 7: Preparation of 2,5-dimethoxy-3-pentyl-6-vinylpyrazine

To a solution of 2-bromo-3,6-dimethoxy-5-pentylpyrazine (1 g, 3.46 mmol, 1 eq) in dioxane (10 mL) and water (1 mL) was added vinylboronic acid pinacol ester (1.60 g, 10.37 mmol, 1.76 mL, 3 eq) and K₂CO₃ (1.43 g, 10.37 mmol, 3 eq) under N₂ atmosphere. The mixture was stirred at 20° C. for 10 min. Then Pd(dppf)Cl₂ (253.04 mg, 345.82 μmol, 0.1 eq) was added and the mixture was stirred at 90° C. for 12 h. On completion, the aqueous phase was extracted with ethyl acetate (20 mL×3). The combined organic phase was washed with brine (20 mL), dried with anhydrous Na₂SO₄, filtered, and concentrated in vacuo. The residue was purified by column chromatography (SiO₂, petroleum ether/ethyl acetate gradient, 100:1 to 50:1) to afford 2,5-dimethoxy-3-pentyl-6-vinylpyrazine (400 mg, 1.69 mmol, 48.95% yield, 100% purity) as a colorless oil. ¹H NMR (400 MHz, CHLOROFORM-d) δ ppm 6.98 (dd, J=10.9, 17.4 Hz, 1H), 6.32 (dd, J=2.1, 17.3 Hz, 1H), 5.44 (dd, J=2.1, 10.9 Hz, 1H), 3.95 (s, 6H), 2.72 (t, J=7.6 Hz, 2H), 1.75-1.64 (m, 2H), 1.40-1.33 (m, 4H), 0.91 (t, J=6.8 Hz, 3H).

### Step 8: Preparation of 3,6-dimethoxy-5-pentylpyrazine-2-carbaldehyde

To a solution of 2,5-dimethoxy-3-pentyl-6-vinylpyrazine (400 mg, 1.69 mmol, 1 eq) in water (2.5 mL) and THF (10 mL) was added OsO₄ (21.52 mg, 84.63 μmol, 4.39 μL, 0.05 eq) at 0° C. Then NaIO₄ (905.13 mg, 4.23 mmol, 234.49 μL, 2.5 eq) in water (5 mL) was added dropwise. The mixture was stirred at 20° C. for 4 h. On completion, water (10 mL) was added, and the mixture was extracted with ethyl acetate (5 mL×3). The combined organic layers were washed with water and dried over Na₂SO₄. The residue was purified by column chromatography (SiO₂, petroleum ether/ethyl acetate gradient, 100:1 to 50:1) to afford 3,6-dimethoxy-5-pentylpyrazine-2-carbaldehyde (350 mg, 1.40 mmol, 82.44% yield, 95% purity) as a yellow solid. ¹H NMR (400 MHz, CHLOROFORM-d) δ ppm 10.20 (s, 1H), 4.07 (s, 3H), 4.01 (s, 3H), 2.86-2.77 (m, 2H), 1.76 (quin, J=7.4 Hz, 2H), 1.42-1.35 (m, 4H), 0.97-0.89 (m, 3H).

### Step 9: Preparation of (E)-2,5-dimethoxy-3-(2-nitrobut-1-en-1-yl)-6-pentylpyrazine

To a solution of 3,6-dimethoxy-5-pentylpyrazine-2-carbaldehyde (100 mg, 419.67 μmol, 1 eq) in nitropropane (3 mL) was added ammonium acetate (32.35 mg, 419.67 μmol, 1 eq) under N₂ atmosphere. The mixture was stirred at 90° C. for 12 h. On completion, the mixture was filtered and concentrated under reduced pressure to give a residue that was purified by prep-TLC (SiO₂, petroleum ether/ethyl acetate gradient=10:1) to afford (E)-2,5-dimethoxy-3-(2-nitrobut-1-en-1-yl)-6-pentylpyrazine (85 mg, 274.76 μmol, 65.47% yield, 100% purity) as a yellow oil. ¹H NMR (400 MHz, CHLOROFORM-d) δ ppm 8.20 (s, 1H), 3.99 (s, 3H), 3.94 (s, 3H), 3.33 (q, J=7.3 Hz, 2H), 2.77 (t, J=7.6 Hz, 2H), 1.74 (quin, J=7.2 Hz, 2H), 1.43-1.34 (m, 4H), 1.26 (t, J=7.3 Hz, 3H), 0.92 (t, J=6.8 Hz, 3H).

### Step 10: Preparation of 2,5-dimethoxy-3-(2-nitrobutyl)-6-pentylpyrazine

To a solution of (E)-2,5-dimethoxy-3-(2-nitrobut-1-en-1-yl)-6-pentylpyrazine (50 mg, 161.62 mol, 1 eq) in THE (1 mL) and MeOH (2 mL) was added NaBH₄ (18.34 mg, 484.87 mol, 3 eq) at 0° C. The mixture was stirred at 20° C. for 5 h. On completion, the reaction mixture was cooled to 0° C. Aqueous NH₄Cl (5 mL) was slowly added to the mixture, and the mixture was extracted with ethyl acetate (20 mL). The extract was washed with water (20 mL) and brine (10 mL), dried over anhydrous Na₂SO₄, filtered, and the filtrate was concentrated under reduced pressure to afford 2,5-dimethoxy-3-(2-nitrobutyl)-6-pentylpyrazine (50 mg, crude) as a colorless oil.

### Step 11: Preparation of 1-(3,6-dimethoxy-5-pentylpyrazin-2-yl)butan-2-amine (12) hydrochloride

To a solution of 2,5-dimethoxy-3-(2-nitrobutyl)-6-pentylpyrazine (50 mg, 160.58 μmol, 1 eq) in EtOH (2 mL) and water (0.5 mL) was added aq. HCl (12 M, 40.14 μL, 3 eq) and the mixture was stirred at 20° C. for 10 min. Then iron powder (˜200 mesh, 44.84 mg, 802.89 μmol, 5 eq) was added and the mixture was stirred at 20° C. for 8 h. On completion, the mixture was filtered and concentrated under reduced pressure to give a residue that was purified by prep-HPLC (column: Phenomenex Luna 80×30 mm×3 μm; mobile phase: [water (HCl)−ACN]; B %: 15%-45%, 8 min) to afford Compound 12 HCl (2.56 mg, 9.10 μmol, 5.67% yield, 100% purity) as a white solid. LC-MS(R_{T}=2.382 min, MS cal.: 281.39, [M+H]⁺ found=282.1); ¹H NMR (400 MHz, CD₃OD) δ=3.94 (s, 3H), 3.93 (s, 3H), 3.70-3.61 (m, 1H), 3.14-3.06 (m, 1H), 3.00-2.91 (m, 1H), 2.72 (t, J=7.6 Hz, 2H), 1.78-1.66 (m, 4H), 1.41-1.32 (m, 4H), 1.08 (t, J=7.5 Hz, 3H), 0.95-0.89 (m, 3H).

### Examples 13-15

Compounds 13-15 are prepared using the teachings herein, analogous methods to the procedures described herein, and knowledge of one of ordinary skill in the art.

### Example 16: Preparation of 2-(5-chloro-3,6-dimethoxypyridin-2-yl)ethan-1-amine (16) hydrochloride

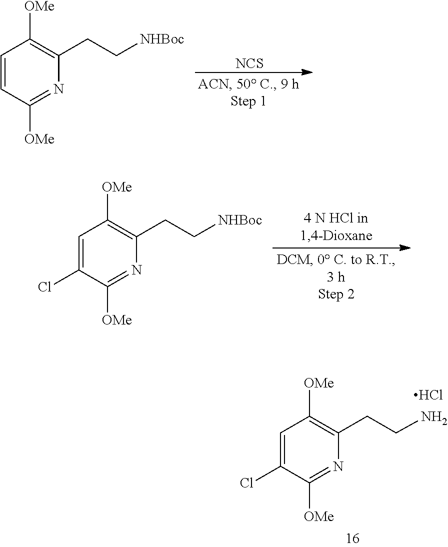 [SMILES: COc1ccc(OC)c(CCNC(=O)OC(C)(C)C)n1 ; "NCS" ; COc1cc(Cl)c(OC)nc1CCNC(=O)OC(C)(C)C ; COc1cc(Cl)c(OC)nc1CCN ; Cl]

### Step 1: Preparation of tert-butyl (2-(5-chloro-3,6-dimethoxypyridin-2-yl)ethyl)carbamate

To a stirred solution of tert-butyl (2-(3,6-dimethoxypyridin-2-yl)ethyl)carbamate (0.100 g, 0.354 mmol) in dry acetonitrile (5 mL) at 0° C., was added N-chlorosuccinimide (0.071 g, 0.531 mmol) in small portions over 3 min. After completion of the addition, the reaction mixture was heated at 50° C. for 9 h. Progress of reaction was monitored by LC-MS. Once complete, the reaction mixture was cooled to room temperature. The reaction was quenched by addition of water (10 mL), and the mixture was extracted with ethyl acetate (2×25 mL). The combined organic extract was washed with brine (20 mL), dried over anhydrous sodium sulfate, and concentrated under reduced pressure. This crude compound was purified by column chromatography (SiO₂, 10% Ethyl acetate/Hexane) desired fraction was concentrated under reduced pressure to afford tert-butyl (2-(5-chloro-3,6-dimethoxypyridin-2-yl)ethyl)carbamate as a light-brown sticky solid (Yield: 0.055 g, 49.02%). HRMS m/z 317.10 [M+1]+.

### Step 2: Preparation of 2-(5-chloro-3,6-dimethoxypyridin-2-yl)ethan-1-amine (16) hydrochloride

To a stirred solution of tert-butyl (2-(5-chloro-3,6-dimethoxypyridin-2-yl)ethyl)carbamate (0.055 g, 0.174 mmol in dichloromethane (2 mL) at 0° C. was added 4 N hydrogen chloride in 1,4-dioxane (2 mL) over a period of 5 min. The reaction mixture was stirred at room temperature for 3 h. After completion, the reaction mixture was concentrated under reduced pressure. This crude material was triturated by using n-pentane (3 mL) and diethyl ether (1 mL) to afford Compound 16 HCl as an off-white solid (Yield: 0.041 g, 93%). HRMS m/z 217.15 [M+1]+; ¹H NMR (400 MHz, DMSO-d₆) δ=7.74 (br s, 3H) 7.71 (s, 1H), 3.92 (s, 3H), 3.77 (s, 3H), 3.19-3.18 (m, 2H), 2.95 (t, J=7.2 Hz, 2H).

### Example 17: Preparation of 1-(5-chloro-3,6-dimethoxypyridin-2-yl) propan-2-amine (17) hydrochloride

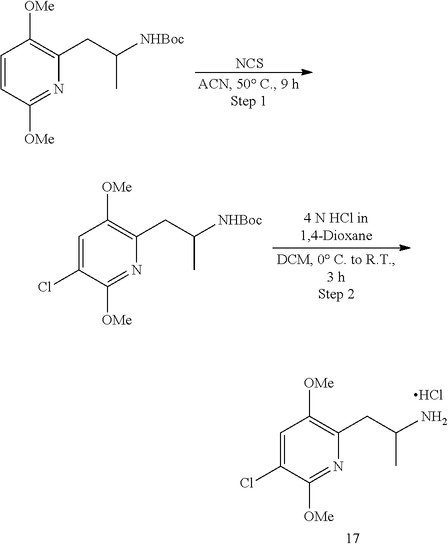 [SMILES: COc1ccc(OC)c(CC(C)NC(=O)OC(C)(C)C)n1 ; "NCS" ; COc1cc(Cl)c(OC)nc1CC(C)NC(=O)OC(C)(C)C ; COc1cc(Cl)c(OC)nc1CC(C)N ; Cl]

### Step 1: Preparation of tert-butyl (1-(5-chloro-3,6-dimethoxypyridin-2-yl)propan-2-yl)carbamate

A stirred solution of tert-butyl (1-(3,6-dimethoxypyridin-2-yl)propan-2-yl)carbamate (150 mg, 0.506 mmol, 1.0 eq.) in dry acetonitrile (5 mL) at 0° C., was treated with N-chlorosuccinimide (101 mg, 1.5 eq, 0.759 mmol) in a portion-wise fashion over 3 min. After completion, the reaction mixture was heated at 50° C. for 9 h. The progress of reaction was monitored by LC-MS. Once complete, the reaction mixture was cooled to room temperature. The reaction quenched with water (10 mL) and extracted with ethyl acetate (2×25 mL). The combined organic extract was washed with brine (20 mL), dried over anhydrous sodium sulfate, and concentrated under reduced pressure to afford crude residue. This crude material was purified by column chromatography (SiO₂, 10% Ethyl acetate/Hexane) desired fraction was concentrated under reduced pressure to afford tert-butyl (1-(5-chloro-3,6-dimethoxypyridin-2-yl)propan-2-yl)carbamate as a light-brown sticky compound (Yield: 0.080 g, 47.78%). HRMS m/z 331.15 [M+1]+.

### Step 2: Preparation of 1-(5-chloro-3,6-dimethoxypyridin-2-yl)propan-2-amine (17) hydrochloride

To a stirred solution of tert-butyl (1-(5-chloro-3,6-dimethoxypyridin-2-yl)propan-2-yl)carbamate (0.055 g, 0.166 mmol) in dichloromethane (2 mL) at 0° C. was added 4 N hydrogen chloride in 1,4-dioxane (3 mL) over a period of 5 min. The reaction mixture was stirred at room temperature for 3 h. Once complete, the reaction mixture was concentrated under reduced pressure. This crude material was triturated by using n-pentane (2 mL) and diethyl ether (1 mL) to afford Compound 17 HCl as an off-white solid (Yield: 0.038 g, 85%). HRMS m/z 231.10 [M+1]⁺; ¹H NMR (400 MHz, DMSO-d₆) δ=9.68 (br s, 1H), 7.76 (3H), 7.72 (s, 1H), 3.89 (s, 3H), 3.80 (s, 3H), 3.74-3.62 (m, 1H), 2.90-2.85 (m, 2H), 1.19 (d, J=4 Hz, 3H).

### Example 18: Preparation of 1-(5-chloro-3,6-dimethoxypyridin-2-yl)butan-2-amine (18) hydrochloride

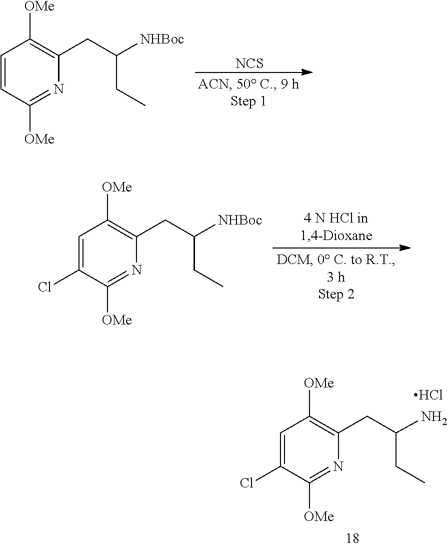 [SMILES: CCC(Cc1nc(OC)ccc1OC)NC(=O)OC(C)(C)C ; "NCS" ; CCC(Cc1nc(OC)c(Cl)cc1OC)NC(=O)OC(C)(C)C ; CCC(N)Cc1nc(OC)c(Cl)cc1OC ; Cl]

### Step 1: Preparation of tert-butyl (1-(5-chloro-3,6-dimethoxypyridin-2-yl)butan-2-yl)carbamate

A stirred solution of tert-butyl (1-(3,6-dimethoxypyridin-2-yl)butan-2-yl)carbamate (0.150 g, 0.483 mmol) in dry acetonitrile (5 mL) at 0° C. was treated with N-chlorosuccinimide (96.8 mg, 0.725 mmol) portionwise over 3 min. After completion of addition, the reaction mixture was heated at 50° C. for 9 h. Progress of reaction was monitored by LC-MS. Once complete, the reaction mixture was cooled to room temperature. The reaction mixture was quenched with water (10 mL) and extracted with ethyl acetate (2×25 mL). The combined organic extracts were washed with brine (20 mL), dried over anhydrous sodium sulfate, and concentrated under reduced pressure. This crude material was purified by column chromatography (SiO₂, 10% Ethyl acetate/Hexane) desired fraction was concentrated under reduced pressure to afford tert-butyl (1-(5-chloro-3,6-dimethoxypyridin-2-yl)butan-2-yl)carbamate as a white solid (Yield: 0.078 g, 48.01%). HRMS m/z 345.20 [M+1]⁺.

### Step 2: Preparation of 1-(5-chloro-3,6-dimethoxypyridin-2-yl)butan-2-amine (18) hydrochloride

To a stirred solution of tert-butyl (1-(5-chloro-3,6-dimethoxypyridin-2-yl)butan-2-yl)carbamate (0.074 g, 0.215 mmol) in dichloromethane (3 mL) at 0° C. was added 4 N hydrogen chloride in 1,4-dioxane (3 mL) over a period of 5 min. The reaction mixture was stirred at room temperature for 3 h. After completion, the reaction mixture was concentrated under reduced pressure to afford crude residue. The crude material was triturated by using n-pentane (2 mL) and diethyl ether (1 mL) to afford Compound 18 HCl as a colorless sticky solid (Yield: 0.052 g, 86.18%). HRMS m/z 245.06 [M+1]⁺; ¹HNMR (400 MHz, DMSO-d₆): δ=7.72 (s, 3H), 3.90 (s, 3H), 3.80 (s, 3H), 3.58-3.53 (m, 1H), 2.99-2.84 (m, 2H), 1.61-1.54 (m, 2H), 0.94 (t, J=7.4 Hz, 3H).

### Example 19: Preparation of 2-(5-ethyl-3,6-dimethoxypyridin-2-yl)ethan-1-amine (19) hydrochloride

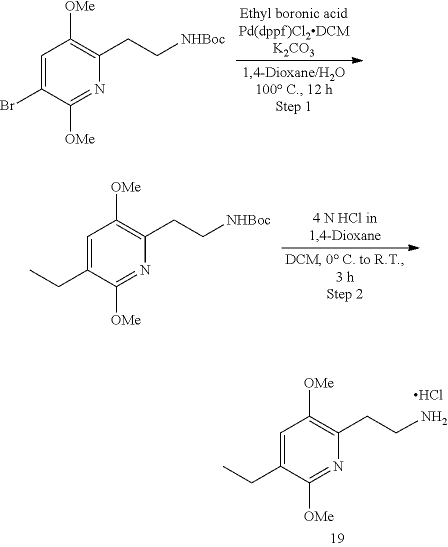 [SMILES: COc1cc(Br)c(OC)nc1CCNC(=O)OC(C)(C)C ; CCc1cc(OC)c(CCNC(=O)OC(C)(C)C)nc1OC ; CCc1cc(OC)c(CCN)nc1OC ; Cl]

### Step 1: Preparation of tert-butyl (2-(5-ethyl-3,6-dimethoxypyridin-2-yl)ethyl)carbamate

A stirred solution of tert-butyl (2-(5-bromo-3,6-dimethoxypyridin-2-yl)ethyl)carbamate (0.150 g, 0.415 mmol) in 1,4-dioxane/water (5.0 mL of a 7:3 v/v mixture) was treated with ethylboronic acid (0.061 g, 0.831 mmol) and potassium carbonate (0.172 g, 1.25 mmol). The mixture was purged with nitrogen gas for 10 min, and then PdCl₂(dppf)·DCM (0.034 g 0.041 mmol) was added to the reaction mixture under nitrogen atmosphere. The reaction mixture was then heated to 100° C. for 12 h and was monitored by TLC and LC-MS. Once complete the reaction mixture was cooled to room temperature, diluted with ethyl acetate (10 mL), passed through Celite pad to remove the catalyst, and washed with ethyl acetate (2×10 mL). The combined organic layer was diluted with water (10 mL) and the aqueous layer was extracted with ethyl acetate (2×25 mL). The combined organic extracts were washed with brine (20 mL), dried over anhydrous Na₂SO₄, filtered, and concentrated under reduced pressure. This crude compound was purified by column chromatography (SiO₂, 10% Ethyl acetate/Hexane) and desired fractions were concentrated under reduced pressure to afford tert-butyl (2-(5-ethyl-3,6-dimethoxypyridin-2-yl)ethyl)carbamate as a white solid (Yield: 0.050 g, 39%). HRMS m/z 311.05 [M+1]⁺.

### Step 2: Preparation of 2-(5-ethyl-3,6-dimethoxypyridin-2-yl)ethan-1-amine (19) hydrochloride

To a stirred solution of tert-butyl (2-(5-ethyl-3,6-dimethoxypyridin-2-yl)ethyl)carbamate (0.050 g, 0.161 mmol) in dichloromethane (2 mL) at 0° C. was added 4 N hydrogen chloride in 1,4-dioxane (2 mL) over a period of 5 min. The reaction mixture was stirred at room temperature for 3 h. After completion, the mixture was concentrated under reduced pressure to afford a crude residue. The product was isolated after triturated with n-pentane (2 mL) and diethyl ether (1 mL) to afford Compound 19 HCl as an off-white solid (Yield: 0.030 g, 75%). HRMS m/z 211.15 [M+1]⁺; ¹H NMR (400 MHz, DMSO-d₆) δ ppm=7.89 (br s, 3H), 7.33 (s, 1H), 3.87 (s, 3H), 3.77 (s, 311), 3.19-3.14 (m, 2H), 2.93 (t, J=7.6 Hz, 2H), 2.55 (m, 2H), 1.13 (t, J=7.6 Hz, 3H).

### Example 20: Preparation of 1-(5-ethyl-3,6-dimethoxypyridin-2-yl)propan-2-amine (20) hydrochloride

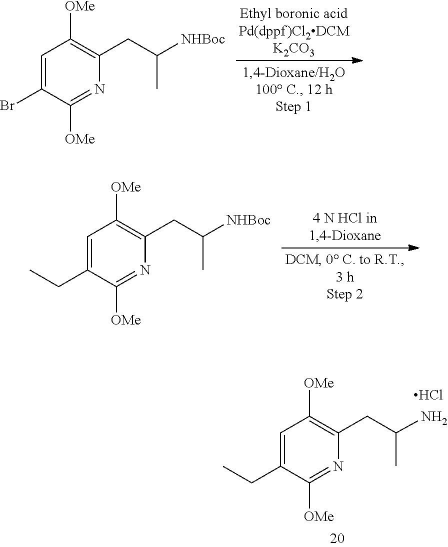 [SMILES: COc1cc(Br)c(OC)nc1CC(C)NC(=O)OC(C)(C)C ; CCc1cc(OC)c(CC(C)NC(=O)OC(C)(C)C)nc1OC ; CCc1cc(OC)c(CC(C)N)nc1OC ; Cl]

### Step 1: Preparation of tert-butyl (1-(5-ethyl-3,6-dimethoxypyridin-2-yl)butan-2-yl)carbamate

To a stirred solution of tert-butyl (1-(5-bromo-3,6-dimethoxypyridin-2-yl)propan-2-yl)carbamate (0.150 g, 0.4 mmol), in 1,4-dioxane/water (5.0 mL of a 7:3 v/v mixture) was added ethylboronic acid (0.074 g, 0.999 mmol) and potassium carbonate (0.166 g, 1.2 mmol). The mixture was purged with nitrogen for 10 min, and then treated with PdC12(dppf)·DCM (0.033 g, 0.044 mmol) under nitrogen atmosphere followed by heating to 100° C. for 12 h. The reaction progress was monitored by TLC and LC-MS. Once complete, the reaction mixture was cooled to room temperature and then diluted with ethyl acetate (10 mL). The mixture was passed through a Celite pad to remove the catalyst and the Celite was washed with ethyl acetate (2×10 mL). The combined organic layer was diluted with water (10 mL) and the aqueous phase was extracted with ethyl acetate (2×25 mL). The organic extracts were washed with brine (20 mL), then dried over anhydrous Na₂SO₄, filtered, and concentrated under reduced pressure. This crude material was purified by column chromatography (SiO₂, 10% Ethyl acetate/Hexane) and desired fractions were concentrated under reduced pressure to afford tert-butyl (1-(5-ethyl-3,6-dimethoxypyridin-2-yl)propan-2-yl)carbamate as an off-white solid (Yield: 0.055 g, 42%). HRMS m/z 325.30 [M+1]⁺.

### Step 2: Preparation of 1-(5-ethyl-3,6-dimethoxypyridin-2-yl)propan-2-amine (20) hydrochloride

To a stirred the solution of tert-butyl (1-(5-ethyl-3,6-dimethoxypyridin-2-yl)propan-2-yl)carbamate (0.055 g, 0.169 mmol) in dichloromethane at 0° C. (2 mL) was added 4 N hydrogen chloride in 1,4-dioxane (2 mL) over a period of 5 min. The reaction mixture was stirred at room temperature for 3 h. After completion, the reaction mixture was concentrated under reduced pressure. This crude material was triturated by using n-pentane (2 mL) and diethyl ether (1 mL) to afford Compound 20 HCl as a colorless, sticky solid (Yield: 0.042 g, 95%). HRMS m/z 225.20 [M+1]⁺; ¹H NMR (400 MHz, DMSO-d₆) δ=7.87 (br. s, 3H), 7.35 (s, 1H), 3.83 (s, 3H), 3.77 (s, 3H), 3.70-3.64 (m, 1H), 2.94-2.83 (m, 2H), 2.56-2-52 (m, 2H), 1.29-1.22 (m, 3H), 1.18-1.12 (M, 3H).

### Example 21: Preparation of 1-(5-ethyl-3,6-dimethoxypyridin-2-yl)butan-2-amine (21) hydrochloride

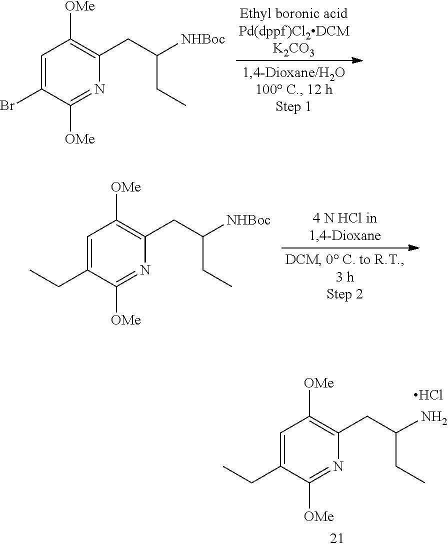 [SMILES: CCC(Cc1nc(OC)c(Br)cc1OC)NC(=O)OC(C)(C)C ; CCc1cc(OC)c(CC(CC)NC(=O)OC(C)(C)C)nc1OC ; CCc1cc(OC)c(CC(N)CC)nc1OC ; Cl]

### Step 1: Preparation of tert-butyl (1-(5-ethyl-3,6-dimethoxypyridin-2-yl)butan-2-yl)carbamate

To a stirred solution of tert-butyl (1-(5-bromo-3,6-dimethoxypyridin-2-yl)butan-2-yl)carbamate (0.130 g, 0.334 mmol), in 1,4-dioxane/water (5.0 mL of a 7:3 v/v mixture) was added ethylboronic acid (60.95 mg, 2.0 eq, 0.664 mmol) and potassium carbonate (0.138 g, 1.002 mmol). The mixture was purged with nitrogen for 10 min, and then PdCl₂(dppf)·DCM (27.25 mg, 0.0334 mmol) was added to the reaction mixture under nitrogen atmosphere followed by heating to 100° C. for 12 h. The reaction progress was monitored by TLC and LC-MS. Once complete the reaction mixture was cooled to room temperature and was diluted with ethyl acetate (10 mL), passed through Celite pad to remove the catalyst and the Celite was washed with ethyl acetate (2×10 mL). The combined organic fraction was diluted with water (10 mL) and the aqueous layer was extracted with ethyl acetate (2×25 mL). The combined organic extracts were washed with brine (20 mL), dried over anhydrous Na₂SO₄, filtered, and concentrated under reduced pressure. This crude compound was purified by column chromatography (SiO₂, 10% Ethyl acetate/Hexane) and desired fractions were concentrated under reduced pressure to afford tert-butyl (1-(5-ethyl-3,6-dimethoxypyridin-2-yl)butan-2-yl)carbamate as an off-white solid (Yield: 0.060 g, 46%). HRMS m/z 339.15 [M+1]⁺.

### Step 2: Preparation of 1-(5-ethyl-3,6-dimethoxypyridin-2-yl)butan-2-amine (21) hydrochloride

To a stirred solution of tert-butyl (1-(5-ethyl-3,6-dimethoxypyridin-2-yl)butan-2-yl)carbamate (0.060 g, 0.177 mmol) in dichloromethane (2 mL) at 0° C. was added 4 N hydrogen chloride in 1,4-dioxane (3 mL) over a period of 5 min. The reaction mixture was stirred at room temperature for 3 h. After completion, the reaction mixture was concentrated under reduced pressure. This crude material was triturated by using n-pentane (2 mL) and diethyl ether (1 mL) to afford Compound 21 HCl as an off-white solid (Yield: 0.043 g, 88.08%). HRMS m/z 239.20 [M+1]⁺; ¹H NMR (400 MHz, DMSO-d₆): δ=7.73 (br s, 3H), 7.35 (s 1H), 3.84 (s, 3H), 3.77 (s, 3H), 3.54-3.53 (m, 1H), 2.96-2.91 (m, 1H), 2.86-2.80 (m, 1H), 2.54 (m, 2H), 1.60-1.53 (m, 2H), 1.46 (t, J=7.6 Hz, 3H), 0.95 (t, J=7.6 Hz, 3H).

### Example 22: Preparation of 2-(5-(ethylthio)-3,6-dimethoxypyridin-2-yl)ethan-1-amine (22) hydrochloride

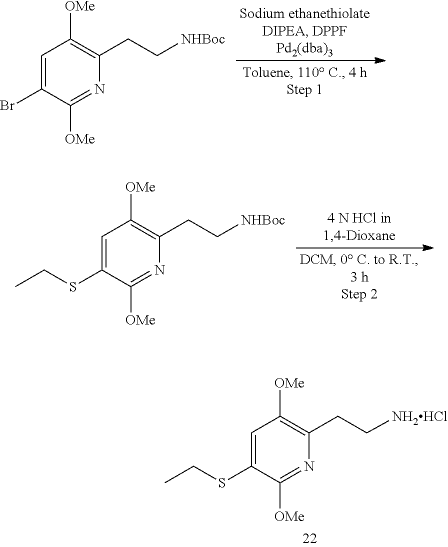

### Step 1: Preparation of tert-butyl (2-(5-(ethylthio)-3,6-dimethoxypyridin-2-yl)ethyl)carbamate

To a stirred solution of tert-butyl (2-(5-bromo-3,6-dimethoxypyridin-2-yl)ethyl)carbamate (0.200 g, 0.554 mmol), in toluene (3 mL) was added sodium ethanethiolate (0.045 g, 0.720 mmol) and DIPEA (0.29 mL, 1.66 mmol). The reaction mixture was purged with nitrogen for 10 min, and then DPPF (0.030 g, 0.055 mmol) and Pd₂(dba)₃ (0.050 g, 0.055 mmol) were added to the reaction mixture under nitrogen atmosphere. The reaction mixture was heated to 110° C. for 4 h. Reaction progress was monitored by TLC and LC-MS. Once complete, the reaction mixture was cooled to room temperature and diluted with ethyl acetate (10 mL). The mixture was passed through a Celite pad to remove the catalyst and the Celite was washed with ethyl acetate (2×10 mL). To the organic layer was added water (10 mL) and the aqueous phase was extracted with ethyl acetate (2×25 mL). The combined organic extracts were washed with brine (20 mL), and then dried over anhydrous Na₂SO₄, filtered, and concentrated under reduced pressure. This crude material was purified by column chromatography (SiO₂, 10% Ethyl acetate/Hexane) and desired fractions were concentrated under reduced pressure to afford tert-butyl (2-(5-(ethylthio)-3,6-dimethoxypyridin-2-yl)ethyl)carbamate as a yellow solid (Yield: 0.120 g, 63.29%). HRMS m/z 343.15 [M+1]⁺.

### Step 2: Preparation of 2-(5-(ethylthio)-3,6-dimethoxypyridin-2-yl)ethan-1-amine (22) hydrochloride

To a stirred solution of tert-butyl (2-(5-(ethylthio)-3,6-dimethoxypyridin-2-yl)ethyl)carbamate (0.100 g, 0.292 mmol) in dichloromethane (3 mL) at 0° C. was added 4 N hydrogen chloride in 1,4-dioxane (3 mL) over a period of 5 min. The reaction mixture was stirred at room temperature for 3 h. After completion, the reaction mixture was concentrated under reduced pressure. This crude material was triturated by using n-pentane (3 mL) and diethyl ether (1.5 mL) to afford Compound 22 HCl as an off-white solid (Yield: 70 mg, 86% yield). HRMS m/z 243.15 [M+1]⁺; ¹H NMR (400 MHz, DMSO-d₆) δ=7.86 (br s, 3H), 7.31 (s, 1H), 3.85 (s, 3H), 3.81 (s, 3H), 3.19-3.17 (m, 2H), 3.01-2.92 (m, 4H), 1.23 (t, J=7.6 Hz, 3H).

### Example 23: Preparation of 1-(5-(ethylthio)-3,6-dimethoxypyridin-2-yl)propan-2-amine (23) hydrochloride (23)

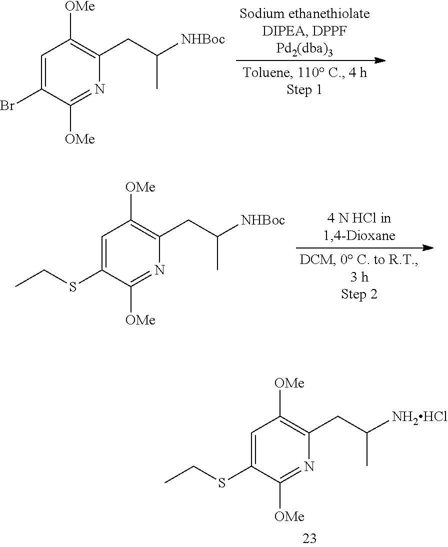

### Step 1: Preparation of tert-butyl (1-(5-(ethylthio)-3,6-dimethoxypyridin-2-yl)propan-2-yl)carbamate

To a stirred solution of tert-butyl (1-(5-bromo-3,6-dimethoxypyridin-2-yl)propan-2-yl)carbamate (0.1 g, 0.266 mmol), and sodium ethanethiolate (0.021 g, 0.346 mmol) in toluene (3 mL) was added DIPEA (0.15 mL, 0.799 mmol). The reaction mixture was purged with nitrogen for 10 min. DPPF (0.015 mg, 0.026 mmol) and Pd₂(dba)₃ (0.024 g, 0.027 mmol) were added to the reaction mixture under nitrogen atmosphere, and then the reaction mixture was heated to 110° C. for 4 h. The reaction progress was monitored by TLC and LC-MS. Once complete, the reaction mixture was cooled to room temperature and diluted with ethyl acetate (10 mL). The mixture was passed through Celite pad to remove the catalyst and the pad was washed with ethyl acetate (2×10 mL). The combined organic layer was diluted with water (10 mL) and the aqueous layer was extracted with ethyl acetate (2×25 mL). The combined organic extracts were washed with brine (20 mL), and then dried over anhydrous Na₂SO₄, filtered, and concentrated under reduced pressure. This crude compound was purified by column chromatography (SiO₂, 10% Ethyl acetate/Hexane) and desired fractions were concentrated under reduced pressure to afford tert-butyl (1-(5-(ethylthio)-3,6-dimethoxypyridin-2-yl)propan-2-yl)carbamate as a yellow solid (Yield: 0.070 g, 73.69%). HRMS m/z 357.10 [M+1]⁺.

### Step 2: Preparation of 1-(5-(ethylthio)-3,6-dimethoxypyridin-2-yl)propan-2-amine (23) hydrochloride

To a stirred solution of tert-butyl (1-(5-(ethylthio)-3,6-dimethoxypyridin-2-yl)propan-2-yl)carbamate (0.070 g, 0.196 mmol) in dichloromethane (3 mL) at 0° C. was added 4 N hydrogen chloride in 1,4-dioxane (3 mL) over a period of 5 min. The reaction mixture was stirred at room temperature for 3 h. After completion, the reaction mixture was concentrated under reduced pressure. This crude compound was triturated by using n-pentane (3 mL) and diethyl ether (1.5 mL) to afford Compound 23 HCl as an off-white sticky solid (Yield: 0.050 g, 87% yield). HRMS m/z 257.20 [M+1]⁺; ¹H NMR (400 MHz, DMSO-d₆) δ=7.84 (br s, 3H), 7.32 (s, 1H), 3.85 (s, 3H), 3.81 (s, 3H), 3.69-3.62 (m, 1H), 3.09-2.96 (m, 2H), 2.90-2.82 (m, 2H), 1.24 (t, J=7.6 Hz, 3H), 1.20-1.18 (d, J=6.8 Hz, 3H).

### Example 24: Preparation of 1-(5-(ethylthio)-3,6-dimethoxypyridin-2-yl)butan-2-amine (24) hydrochloride

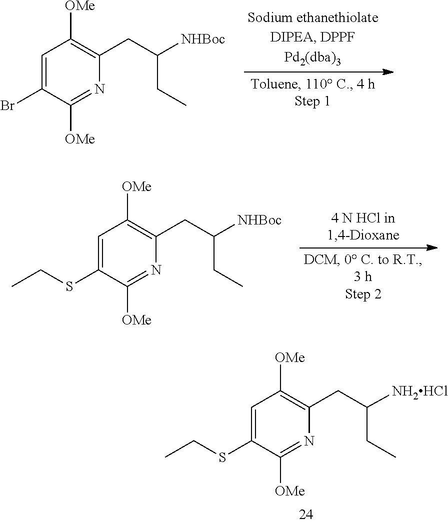

### Step 1: Preparation of tert-butyl (1-(5-(ethylthio)-3,6-dimethoxypyridin-2-yl)butan-2-ylcarbamate

To a stirred solution of tert-butyl (1-(5-bromo-3,6-dimethoxypyridin-2-yl)butan-2-yl)carbamate (0.100 g, 0.257 mmol), and sodium ethanethiolate (0.021 g, 0.334 mmol) in toluene (3 mL) was added DIPEA (99.6 mg, 0.771 mmol). The reaction mixture was purged with nitrogen for 10 min, and then DPPF (0.014 g, 0.0257 mmol) and Pd₂(dba)₃ (0.023 g, 0.0257 mmol) were added under nitrogen atmosphere. The reaction mixture was heated to 110° C. for 4 h. Reaction progress was monitored by TLC and LC-MS. Once complete, the reaction mixture was cooled to room temperature and was then diluted with ethyl acetate (10 mL) and passed through Celite pad to remove the catalyst. The Celite pad was washed with ethyl acetate (2×10 mL). To the combined organic layer was added water (10 mL) and the water extracted with ethyl acetate (2×25 mL). The combined organic extracts were washed with brine (20 mL), dried over anhydrous Na₂SO₄, filtered, and concentrated under reduced pressure. This crude material was purified by column chromatography (SiO₂, 10% Ethyl acetate/Hexane) and desired fractions were concentrated under reduced pressure to afford tert-butyl (1-(5-(ethylthio)-3,6-dimethoxypyridin-2-yl)butan-2-yl)carbamate as a yellow solid (Yield: 0.060 g, 63.04%). HRMS m/z 371.00 [M+1]⁺.

### Step 2: Preparation of 1-(5-(ethylthio)-3,6-dimethoxypyridin-2-yl)butan-2-amine (24) hydrochloride

To a stirred solution of tert-butyl (1-(5-(ethylthio)-3,6-dimethoxypyridin-2-yl)butan-2-yl)carbamate (60 mg, 0.161 mmol, 1) in dichloromethane (3 mL) at 0° C. was added 4 N hydrogen chloride in 1,4-dioxane (3 mL) over a period of 5 min. The reaction mixture was stirred at room temperature for 3 h. After completion, the reaction mixture was concentrated under reduced pressure to afford a crude residue. The crude material was triturated by using n-pentane (2 mL) and diethyl ether (1 mL) to afford Compound 24 HCl as an off-white solid (Yield: 0.042 g, 84.52% yield). HRMS m/z 271.20 [M+1]⁺; ¹H NMR (400 MHz, DMSO-d₆) δ=7.81 (br s, 3H), 7.31 (s, 1H), 3.88-3.85 (s, 3H), 3.81-377 (s, 3H), 3.52-3.50 (m, 1H), 3.03-2.96 (m, 2H), 2.92-2.83 (m, 2H), 1.59-1.55 (m, 2H), 1.24 (t, J=7.6 Hz, 3H), 0.93 (t, J=7.6 Hz, 3H).

### Example 25: Preparation of 2-(5-bromo-3,6-dimethoxypyridin-2-yl)-N-(2-methoxybenzyl)ethan-1-amine (25)

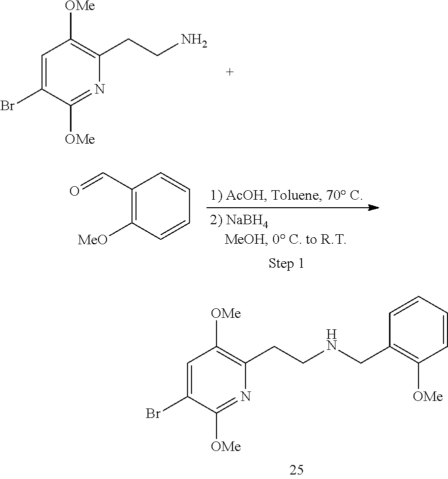

### Step 1: Preparation of 2-(5-bromo-3,6-dimethoxypyridin-2-yl)-N-(2-methoxybenzyl)ethan-1-amine (25)

To a stirred solution of 2-(5-bromo-3,6-dimethoxypyridin-2-yl)ethan-1-amine (0.12 g, 0.459 mmol) and 2-methoxybenzaldehyde (0.075 g, 0.551 mmol) in toluene (5.0 mL) was added a catalytic amount of acetic acid (approximately 75 μl). The reaction mixture, under nitrogen atmosphere, was heated to 70° C. for 12 h. Progress of reaction was monitored by LC-MS. After completion, the reaction mixture was concentrated under reduced pressure afford crude intermediate as an imine. To a stirred solution of the crude imine intermediate in methanol was added sodium borohydride (0.035 g, 0.919 mmol)) at 0° C. and the mixture was stirred for 5 min. The reaction mixture was allowed to warm to room temperature and stirred for 1 h. Reaction progress was monitored by TLC and LC-MS. Once complete, the reaction mixture was diluted with water (10 mL) and extracted with ethyl acetate (2×20 mL). The combined organic extracts were washed with brine (10 mL), dried over anhydrous Na₂SO₄, filtered, and concentrated under reduced pressure. This crude material was purified by column chromatography (SiO₂, 80% Ethyl acetate/Hexane) and the desired fractions were concentrated under reduced pressure to afford Compound 25 as a white solid (Yield: 0.46 g, 26%). HRMS m/z 381.15 [M+1]⁺; ¹H NMR (400 MHz, DMSO-d₆) δ=7.70 (s, 111), 7.25-7.16 (m, 2H), 6.92 (d, J=8.0 Hz, 111), 6.88-6.85 (m, 1H), 3.80 (s, 3H), 3.76 (s, 3H), 3.67 (s, 1H), 2.86-2.78 (m, 2H).

### Example 26: Preparation of 2-(5-cyclopropyl-3,6-dimethoxypyridin-2-yl)ethan-1-amine (26) hydrochloride

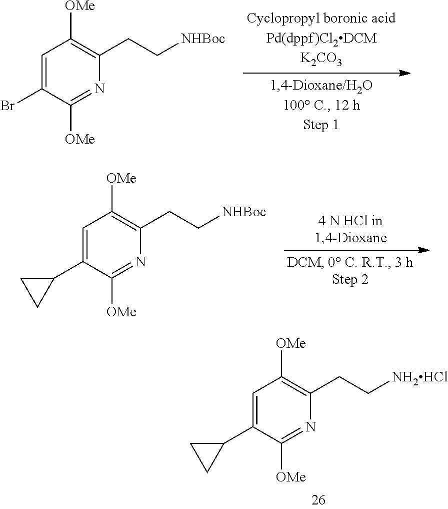

### Step 1: Preparation of tert-butyl (2-(5-cyclopropyl-3,6-dimethoxypyridin-2-yl)ethyl)carbamate

A stirred solution of tert-butyl (2-(5-bromo-3,6-dimethoxypyridin-2-yl)ethyl)carbamate (0.200 g, 0.554 mmol), cyclopropylboronic acid (0.095 g, 1.10 mmol) and potassium carbonate (0.230 g, 1.66 mmol) in 1,4-dioxane/water (5.0 mL of a 7:3 v/v mixture) was purged with nitrogen for 10 min. This mixture was treated with PdCl₂(dppf)·DCM (0.045 g, 0.055 mmol) under nitrogen atmosphere and then heated to 100° C. for 12 h. The reaction progress was monitored by TLC and LC-MS. Once complete, the reaction mixture was cooled to room temperature and then diluted with ethyl acetate (10 mL), passed through Celite pad to remove the catalyst and the Celite was washed with ethyl acetate (2×10 mL). The combined organic layers were diluted with water (10 mL) and the aqueous phase was re-extracted with ethyl acetate (2×25 mL). The combined organic extracts were washed with brine (20 mL), dried over anhydrous Na₂SO₄, filtered, and concentrated under reduced pressure. This crude material was purified by column chromatography (SiO₂, 10% Ethyl acetate/Hexane) and desired fractions were concentrated under reduced pressure to afford tert-butyl (2-(5-cyclopropyl-3,6-dimethoxypyridin-2-yl)ethyl)carbamate as an off-white solid (Yield: 0.150 g, 84%). HRMS m/z 323.20 [M+1]⁺.

### Step 2: Preparation of 2-(5-cyclopropyl-3,6-dimethoxypyridin-2-yl)ethan-1-amine (26) hydrochloride

To a stirred solution of tert-butyl (2-(5-cyclopropyl-3,6-dimethoxypyridin-2-yl)ethyl)carbamate (0.150 g, 0.465 mmol) in dichloromethane (2 mL) at 0° C. was added 4 N hydrogen chloride in 1,4-dioxane (3 mL) over a period of 5 min. The reaction mixture was stirred at room temperature for 3 h. After completion, the reaction mixture was concentrated under reduced pressure. This crude material was triturated by using n-pentane (4 mL) and diethyl ether (2 mL) to afford Compound 26 HCl as an off-white solid (Yield: 0.115 g, 91%). HRMS m/z 223.20 [M+1]⁺; ¹H NMR (400 MHz, DMSO-d₆) δ=7.86 (br s, 3H), 6.95 (s, 1H), 3.85 (s, 3H), 3.75 (s, 3H), 3.18-3.13 (m, 2H), 2.91 (t, J=7.2 Hz, 2H), 2.02-1.95 (m, 1H), 0.92-0.89 (m, 2H), 0.78-0.73 (m, 2H).

### Example 27: Preparation of 1-(5-cyclopropvl-3,6-dimethoxypyridin-2-yl)propan-2-amine (27) formate

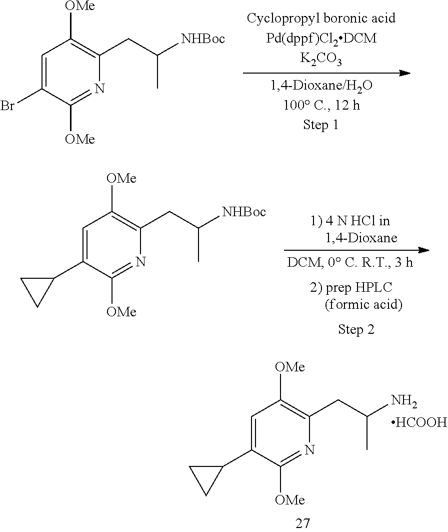 [SMILES: COc1cc(Br)c(OC)nc1CC(C)NC(=O)OC(C)(C)C ; COc1cc(C2CC2)c(OC)nc1CC(C)NC(=O)OC(C)(C)C ; COc1cc(C2CC2)c(OC)nc1CC(C)N ; "·HCOOH"]

### Step 1: Preparation of tert-butyl (1-(5-cyclopropyl-3,6-dimethoxypyridin-2-yl)propan-2-yl)carbamate

A stirred solution of tert-butyl (1-(5-bromo-3,6-dimethoxypyridin-2-yl)propan-2-yl)carbamate (0.200 g, 0.533 mmol), cyclopropylboronic acid (0.092 g, 1.07 mmol) and potassium carbonate (0.221 g, 1.6 mmol), in 1,4-dioxane/water (5.0 mL of a 7:3 v/v mixture) was purged with nitrogen for 10 min. The solution was treated with PdCl₂(dppf)·DCM (0.043 g, 0.053 mmol) under nitrogen atmosphere and then heated to 100° C. for 12 h. The progress of the reaction was monitored by TLC and LC-MS. Once complete, the reaction mixture was cooled to room temperature and then diluted with ethyl acetate (10 mL). The mixture was passed through a Celite pad to remove the catalyst and the Celite was washed with ethyl acetate (2×10 mL). The combined organic layers were diluted with water (10 mL) and the aqueous phase extracted with ethyl acetate (2×25 mL). The combined organic extracts were washed with brine (20 mL), dried over anhydrous Na₂SO₄, filtered, and concentrated under reduced pressure. This crude compound was purified by column chromatography (SiO₂, 10% Ethyl acetate/Hexane) and desired fractions were concentrated under reduced pressure to afford tert-butyl (1-(5-cyclopropyl-3,6-dimethoxypyridin-2-yl)propan-2-yl)carbamate as a white solid (Yield: 0.100 g, 56%). HRMS m/z 337.25 [M+1]⁺.

### Step 2: Preparation of 1-(5-cyclopropyl-3,6-dimethoxypyridin-2-yl)propan-2-amine (27) formate

To a stirred solution of tert-butyl (1-(5-cyclopropyl-3,6-dimethoxypyridin-2-yl)propan-2-yl)carbamate (0.100 g, 0.297 mmol) in dichloromethane (3 mL) at 0° C. was added 4 N hydrogen chloride in 1,4-dioxane (3 mL) over a period of 5 min. The reaction mixture was stirred at room temperature for 3 h. After completion, the reaction mixture was concentrated under reduced pressure. This crude compound was purified by preparative HPLC using 0.1% formic acid buffer in water and desired fractions were lyophilized to afford Compound 27 formate as an off-white, sticky solid (Yield: 0.030 g, 43%). HRMS m/z 237.10 [M+1]⁺; ¹H NMR (400 MHz, DMSO-d₆) δ=8.40 (s, 1H), 6.93 (s, 1H), 3.83 (s, 3H), 3.73 (s, 3H), 3.55-3.46 (m, 2H), 2.81-2.75 (m, 2H), 2.01-1.94 (m, 1H), 1.11-1.09 (d, J=6.4 Hz, 3H), 0.92-0.87 (m, 2H), 0.74-0.70 (m, 2H).

### Example 28: Preparation of 1-(5-cyclopropyl-3,6-dimethoxypyridin-2-yl)butan-2-amine (28) hydrochloride

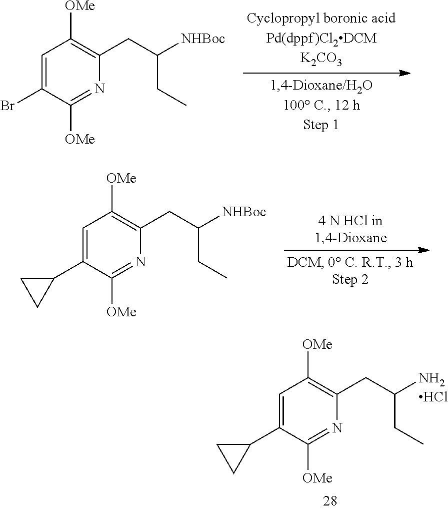 [SMILES: CCC(Cc1nc(OC)c(Br)cc1OC)NC(=O)OC(C)(C)C ; CCC(Cc1nc(OC)c(C2CC2)cc1OC)NC(=O)OC(C)(C)C ; CCC(N)Cc1nc(OC)c(C2CC2)cc1OC ; Cl]

### Step 1: Preparation of tert-butyl (1-(5-cyclopropyl-3,6-dimethoxypyridin-2-yl)butan-2-yl)carbamate

A stirred solution of tert-butyl (1-(5-bromo-3,6-dimethoxypyridin-2-yl)butan-2-yl)carbamate (0.150 g, 0.385 mmol), cyclopropylboronic acid (0.066 g, 0.771 mmol) and postassium carbonate (0.160 g, 1.16 mmol) in 1,4-dioxane/water (5.0 mL of a 7:3 v/v mixture) was purged with nitrogen for 10 min. To this solution was added PdCl₂(dppf)·DCM (0.031 g, 0.0385 mmol) under nitrogen atmosphere followed by heating to 100° C. for 12 h. The reaction progress was monitored by TLC and LC-MS. Once complete, the reaction mixture was cooled to room temperature and then was diluted with ethyl acetate (10 mL). The mixture was passed through a Celite pad to remove the catalyst and the Celite was washed with ethyl acetate (2×10 mL). The combined organic layers were diluted with water (10 mL) and the aqueous layer was extracted with ethyl acetate (2×25 mL). The combined organic extracts were washed with brine (20 mL), dried over anhydrous Na₂SO₄, filtered, and concentrated under reduced pressure. This crude compound was purified by column chromatography (SiO₂, 10% Ethyl acetate/Hexane) and desired fractions were concentrated under reduced pressure to afford tert-butyl (1-(5-cyclopropyl-3,6-dimethoxypyridin-2-yl)butan-2-yl)carbamate as a white solid (Yield: 0.100 g, 74%). HRMS m/z 351.20 [M+1]⁺.

### Step 2: Preparation of 1-(5-cyclopropyl-3,6-dimethoxypyridin-2-yl)butan-2-amine (28) hydrochloride

To a stirred solution of tert-butyl (1-(5-cyclopropyl-3,6-dimethoxypyridin-2-yl)butan-2-yl)carbamate (0.100 g, 0.285 mmol) in dichloromethane (3 mL) at 0° C. was added 4 N HCl in 1,4-dioxane (3 mL) over a period of 5 min. The reaction mixture was stirred at room temperature for 3 h. After completion, the reaction mixture was concentrated under reduced pressure to afford crude residue that was triturated by using n-pentane (3 mL) and diethyl ether (1.5 mL) to afford Compound 28 HCl as an off-white solid (Yield: 0.056 g, 78%). HRMS m/z 251.15 [M+1]⁺; ¹H NMR (400 MHz, DMSO-d₆) δ=77.85 (br s, 3H), 6.96 (s, 1H), 3.80 (s, 3H), 3.75 (s, 3H), 3.55-3.50 (m, 1H), 2.95-2.81 (m, 2H), 2.01-1.96 (m, 1H), 1.60-1.53 (m, 2H), 0.94-0.88 (m, 5H), 0.75-0.71 (m, 2H).

### Example 29: Preparation of 2-(5-hexyl-3,6-dimethoxypyridin-2-yl)ethan-1-amine (29) formate

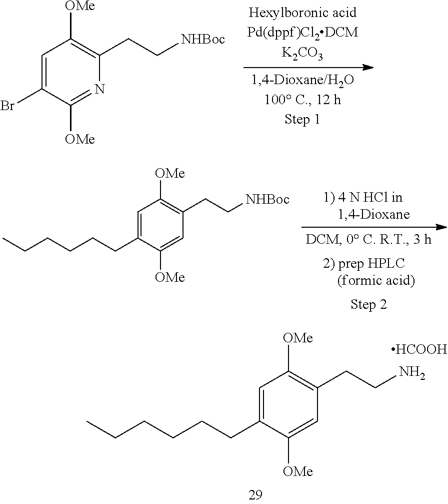 [SMILES: COc1cc(Br)c(OC)nc1CCNC(=O)OC(C)(C)C ; CCCCCCc1cc(OC)c(CCNC(=O)OC(C)(C)C)cc1OC ; CCCCCCc1cc(OC)c(CCN)cc1OC ; "·HCOOH"]

### Step 1: Preparation of tert-butyl (2-(5-hexyl-3,6-dimethoxypyridin-2-yl)ethyl)carbamate

A stirred solution of tert-butyl (2-(5-bromo-3,6-dimethoxypyridin-2-yl)ethyl)carbamate (0.100 g, 0.277 mmol), n-hexylboronic acid (0.072 g, 0.554 mmol) and potassium carbonate (0.115 g, 830 mmol), in 1,4-dioxane/water (5.0 mL of a 7:3 v/v mixture) was purged with nitrogen for 10 min. The solution was treated with PdCl₂(dppf)·DCM (0.022 g, 0.027 mmol) under nitrogen atmosphere and then heated to 100° C. for 12 h. The reaction progress was monitored by TLC and LC-MS. Once complete, the reaction mixture was cooled to room temperature. The reaction mixture was diluted with ethyl acetate (10 mL), passed through Celite pad to remove the catalyst and the Celite pad was washed with ethyl acetate (2×10 mL). The combined organic layers were diluted with water (10 mL) and the aqueous phase was extracted with ethyl acetate (2×25 mL). The combined organic extracts were washed with brine (20 mL), dried over anhydrous Na₂SO₄, filtered, and concentrated under reduced pressure. This crude material was purified by column chromatography (SiO₂, 10% Ethyl acetate/Hexane) and desired fractions were concentrated under reduced pressure to afford tert-butyl (2-(5-hexyl-3,6-dimethoxypyridin-2-yl)ethyl)carbamate as a white solid (Yield: 0.080 g, 79%). HRMS m/z 367.20 [M+1]⁺.

### Step 2: Preparation of 2-(5-hexyl-3,6-dimethoxypyridin-2-yl)ethan-1-amine (29) formate

To a stirred solution of tert-butyl (2-(5-hexyl-3,6-dimethoxypyridin-2-yl)ethyl)carbamate (0.080 g, 0.591 mmol) in dichloromethane (2 mL) at 0° C. was added 4 N HCl in 1,4-dioxane (3 mL) over a period of 5 min. The reaction mixture was stirred at room temperature for 3 h. After completion, the reaction mixture was concentrated under reduced pressure to afford a crude residue. The crude compound was purified by preparative HPLC using 0.1% formic acid buffer in water. The desired fractions were lyophilized to afford Compound 29 formate as an off-white solid (Yield: 0.044 g, 64%). HRMS m/z 267.3 [M+1]⁺; ¹H NMR (400 MHz, DMSO-d₆) δ=8.41 (s, 1H), 7.28 (s, 1H), 3.83 (s, 3H), 3.75 (s, 3H), 3.05 (t, J=7.2 Hz, 2H), 2.85 (t, J=7.2 Hz, 2H), 2.47 (m, 2H), 1.55-1.48 (m, 2H), 1.28-1.27 (s, 6H), 0.87-0.84 (m, 3H).

### Example 30: Preparation of 2-(3,6-dimethoxy-5-(6,6,6-trifluorohexyl)pyridin-2-yl)ethan-1-amine (30) hydrochloride

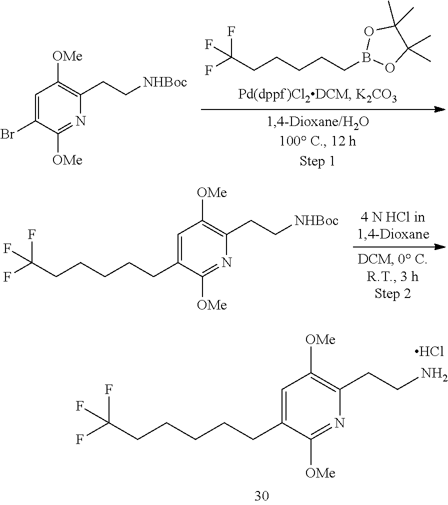 [SMILES: COc1cc(Br)c(OC)nc1CCNC(=O)OC(C)(C)C ; CC1(C)OB(CCCCCC(F)(F)F)OC1(C)C ; COc1cc(CCCCCC(F)(F)F)c(OC)nc1CCNC(=O)OC(C)(C)C ; COc1cc(CCCCCC(F)(F)F)c(OC)nc1CCN ; Cl]

### Step 1a: Preparation of 4,4,5,5-tetramethyl-2-(6,6,6-trifluorohexyl)-1,3,2-dioxaborolane

To a stirred solution of anhydrous copper (I) iodide (0.261 g, 1.37 mmol) in anhydrous DMF (30 mL), was added triphenylphosphine (0.467 g, 1.78 mmol), 4,4,5,5-tetramethyl-2-(4,4,5,5-tetramethyl-1,3,2-dioxaborolan-2-yl)-1,3,2-dioxaborolane (5.2 g, 20.5 mmol) and lithium methoxide (1.04 g, 27.4 mmol) under nitrogen atmosphere at room temperature. The reaction mixture was stirred at room temperature for 5 minutes, and then 6-bromo-1,1,1-trifluorohexane (3.0 g, 13.7 mmol) in anhydrous DMF (5 mL) was added dropwise under nitrogen atmosphere. The reaction mixture was stirred at room temperature for 12 h. The reaction progress was monitored by TLC and LC-MS. Once complete, the reaction mixture was quenched with water (30 mL) and diluted with ethyl acetate (50 mL). The aqueous phase was extracted with ethyl acetate (2×50 mL). The combined organic extracts were washed with brine (30 mL), dried over anhydrous Na₂SO₄, filtered, and concentrated under reduced pressure to afford a crude residue that was purified by column chromatography (SiO₂, 5% Ethyl acetate/Hexane). Desired fractions were concentrated under reduced pressure to afford 4,4,5,5-tetramethyl-2-(6,6,6-trifluorohexyl)-1,3,2-dioxaborolane as a colorless liquid (Yield: 2.5 g, 68.5%). ¹H NMR (400 MHz, CDCl3) δ=2.11-1.99 (m, 2H), 1.60-1.54 (m, 2H), 1.46-1.36 (m, 4H), 1.24 (s, 12H), 0.80 (t, J=6.0 Hz, 2H).

### Step 1: Preparation of tert-butyl (2-(3,6-dimethoxy-5-(6,6,6-trifluorohexyl)pyridin-2-yl)ethyl)carbamate

A stirred solution of tert-butyl (2-(5-bromo-3,6-dimethoxypyridin-2-yl)ethyl)carbamate (0.650 g, 1.8 mmol), 4,4,5,5-tetramethyl-2-(6,6,6-trifluorohexyl)-1,3,2-dioxaborolane (1.42 g, 5.4 mmol) and potassium carbonate (0.746 g, 5.40 mmol) in 1,4-dioxane/water (5.0 mL of a 7:3 v/v mixture) was purged with nitrogen for 10 min. To this solution was added PdCl₂(dppf)·DCM (0.073 g, 0.090 mmol) under nitrogen atmosphere followed by heating to 100° C. for 12 h. The reaction progress was monitored by TLC and LC-MS. Once complete, the reaction mixture was cooled to room temperature and then diluted with ethyl acetate (30 mL). The mixture was passed through a Celite pad to remove the catalyst and the Celite was washed with ethyl acetate (2×20 mL). The combined organic layers were diluted with water (25 mL) and the aqueous phase was extracted with ethyl acetate (2×25 mL). The combined organic extracts were washed with brine (25 mL), dried over anhydrous Na₂SO₄, filtered, and concentrated under reduced pressure to afford a crude residue. The crude compound was purified by column chromatography (SiO₂, 10% Ethyl acetate/Hexane) and desired fractions were concentrated under reduced pressure to afford tert-butyl (2-(3,6-dimethoxy-5-(6,6,6-trifluorohexyl)pyridin-2-yl)ethyl)carbamate as a white solid (Yield: 0.250 g, 33%). HRMS m/z 421.35 [M+1]⁺; ¹H NMR (400 MHz, CDCl3) δ=8.41 (s, 1H), 7.33 (s, 1H), 3.82 (s, 3H), 3.76 (s, 3H), 3.06 (t, J=7.2 Hz, 2H), 2.86 (t, J=7.6 Hz, 2H), 2.60 (m, 2H), 2.32-2.20 (m, 2H), 1.79-1.71 (m, 2H).

### Step 2: Preparation of 2-(3,6-dimethoxy-5-(6,6,6-trifluorohexyl)pyridin-2-yl)ethan-1-amine (30) hydrochloride

To a stirred solution of tert-butyl (2-(3,6-dimethoxy-5-(6,6,6-trifluorohexyl)pyridin-2-yl)ethyl)carbamate (0.250 g, 0.594 mmol) in dichloromethane (4 mL) at 0° C. was added 4 N hydrogen chloride in 1,4-dioxane (4 mL) over a period of 5 min. The reaction mixture was stirred at room temperature for 3 h. After completion, the reaction mixture was concentrated under reduced pressure to afford crude residue that was triturated with n-pentane (4 mL) and diethyl ether (2 mL) to afford Compound 30 HCl as an off-white solid (Yield: 0.140 g, 66%). HRMS m/z 321.2 [M+1]⁺; ¹H NMR (400 MHz, DMSO-d₆) δ=7.88 (br s, 3H), 7.33 (s, 1H), 3.82 (s, 3H), 3.76 (s, 3H), 3.17-3.14 (t, J=7.2 Hz, 2H), 2.93-2.91 (t, J=7.2 Hz, 2H), 2.46 (m, 2H), 2.32-2.11 (m, 2H), 1.55-1.43 (m, 4H), 1.34-1.32 (m, 2H).

### Example 31: Preparation of 2-(5-butyl-3,6-dimethoxypyridin-2-yl)ethan-1-amine (31) hydrochloride

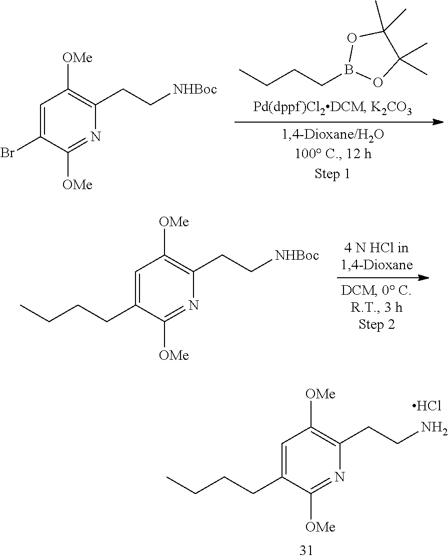 [SMILES: COc1cc(Br)c(OC)nc1CCNC(=O)OC(C)(C)C ; CCCCB1OC(C)(C)C(C)(C)O1 ; CCCCc1cc(OC)c(CCNC(=O)OC(C)(C)C)nc1OC ; CCCCc1cc(OC)c(CCN)nc1OC ; Cl]

### Step 1: Preparation of tert-butyl (2-(5-butyl-3,6-dimethoxypyridin-2-y l)ethyl)carbamate

A stirred solution of tert-butyl (2-(5-bromo-3,6-dimethoxypyridin-2-yl)ethyl)carbamate (0.200 g, 0.554 mmol), 2-butyl-4,4,5,5-tetramethyl-1,3,2-dioxaborolane (0.204 g, 1.11 mmol) and potassium carbonate (0.230 g, 1.66 mmol) in 1,4-dioxane/water (5.0 mL of a 7:3 v/v mixture) was purged with nitrogen for 10 min. The solution was treated with PdCl₂(dppf)·DCM (0.045 g, 0.055 mmol) under nitrogen atmosphere and then the reaction mixture was heated to 100° C. for 12 h. The reaction progress was monitored by TLC and LC-MS. Once complete, the reaction mixture was cooled to room temperature and diluted with ethyl acetate (10 mL). The mixture was passed through Celite pad to remove the catalyst and the Celite was washed with ethyl acetate (2×10 mL). The combined organic layer was diluted with water (10 mL) and the aqueous layer was extracted with ethyl acetate (2×25 mL). The combined organic extracts were washed with brine (20 mL), dried over anhydrous Na₂SO₄, filtered, and concentrated under reduced pressure to afford crude residue that was purified by column chromatography (SiO₂, 10% Ethyl acetate/Hexane). Desired fractions were concentrated under reduced pressure to afford tert-butyl (2-(5-butyl-3,6-dimethoxypyridin-2-yl)ethyl)carbamate as a white solid (Yield: 0.056 g, 30%). HRMS m/z 339.15 [M+1]⁺.

### Step 2: Preparation of 2-(5-butyl-3,6-dimethoxypyridin-2-yl)ethan-1-amine (31) hydrochloride

To a stirred solution of tert-butyl (2-(5-butyl-3,6-dimethoxypyridin-2-yl)ethyl)carbamate (0.056 g, 0.165 mmol) in dichloromethane (2 mL) at 0° C. was added 4 N hydrogen chloride in 1,4-dioxane (3 mL) over a period of 5 min. The reaction mixture was stirred at room temperature for 3 h. After completion, the reaction mixture was concentrated under reduced pressure. This crude compound was triturated by using n-pentane (2 mL) and diethyl ether (1 mL) to afford Compound 31 HCl as an off-white solid (Yield: 0.045 g, 99%). HRMS m/z 239.30 [M+1]⁺; ¹H NMR (400 MHz, DMSO-d₆) δ=7.90 (hr s, 3H), 7.31 (s, 1H), 3.82 (s, 3H), 3.76 (s, 3H), 3.19-3.14 (m, 2H), 2.93 (t, J=7.2 Hz, 2H), 2.52 (m, 2H), 1.54-1.46 (m, 2H), 1.35-1.26 (m, 2H), 0.89 (t, J=7.6 Hz, 3H).

### Example 32: Preparation of 2-(5-(4-fluorobutyl)-3,6-dimethoxypyridin-2-yl)ethan-1-amine (32) hydrochloride

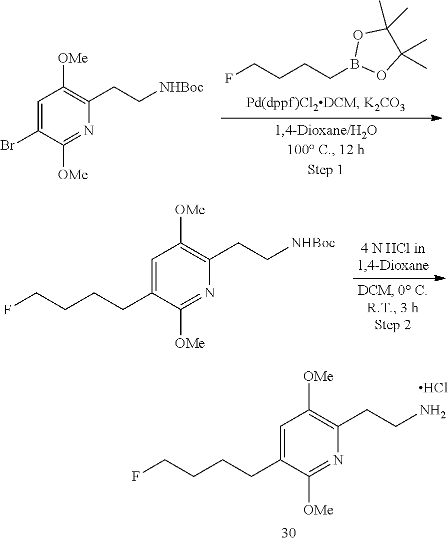 [SMILES: COc1cc(Br)c(OC)nc1CCNC(=O)OC(C)(C)C ; CC1(C)OB(CCCCF)OC1(C)C ; COc1cc(CCCCF)c(OC)nc1CCNC(=O)OC(C)(C)C ; COc1cc(CCCCF)c(OC)nc1CCN ; Cl]

### Step 1: Preparation of tert-butyl (2-(5-(4-fluorobutyl)-3,6-dimethoxypyridin-2-yl)ethyl)carbamate

A stirred solution of tert-butyl (2-(5-bromo-3,6-dimethoxypyridin-2-yl)ethyl)carbamate (0.300 g, 0.830 mmol), 2-(4-fluorobutyl)-4,4,5,5-tetramethyl-1,3,2-dioxaborolane (0.335 g, 1.66 mmol), and potassium carbonate (0.344 g, 2.49 mmol) in 1,4-dioxane/water (5.0 mL of a 7:3 v/v mixture) was purged with nitrogen for 10 min. To this solution was added PdCl₂(dppf)·DCM (0.068 g, 0.083 mmol) under nitrogen atmosphere and the reaction mixture was heated to 100° C. for 12 h. The reaction progress was monitored by TLC and LC-MS. Once complete, the reaction mixture was cooled to room temperature and then diluted with ethyl acetate (10 mL). The mixture was passed through Celite pad to remove the catalyst and the Celite was washed with ethyl acetate (2×10 mL). The combined organic layers were diluted with water (15 mL) and the aqueous phase was extracted with ethyl acetate (2×25 mL). The combined organic extracts were washed with brine (15 mL), dried over anhydrous Na₂SO₄, filtered, and concentrated under reduced pressure to afford a crude residue that was purified by column chromatography (SiO₂, 10% Ethyl acetate/Hexane). The desired fractions were concentrated under reduced pressure to afford tert-butyl (2-(5-(4-fluorobutyl)-3,6-dimethoxypyridin-2-yl)ethyl)carbamate as a white solid (Yield: 0.180 g, 60%). HRMS m/z 357.10 [M+1]⁺.

### Step 2: Preparation of 2-(5-(4-fluorobutyl)-3,6-dimethoxypyridin-2-yl)ethan-1-amine (32) hydrochloride

To a stirred solution of tert-butyl (2-(5-(4-fluorobutyl)-3,6-dimethoxypyridin-2-yl)ethyl)carbamate (0.180 g, 0.510 mmol) in dichloromethane (3 mL) at 0° C. was added 4 N hydrogen chloride in 1,4-dioxane (3 mL) over a period of 5 min. The reaction mixture was stirred at room temperature for 3 h. After completion, the reaction mixture was concentrated under reduced pressure. The crude material was triturated by using n-pentane (2 mL) and diethyl ether (1 mL) to afford Compound 32 HCl as an off-white solid (Yield: 0.060 g, 40%). HRMS m/z 257.3 [M+1]⁺; ¹H NMR (400 MHz, DMSO-d₆) δ=7.85 (br s, 3H), 7.34 (s, 1H), 4.51 (t, J=6 Hz, 1H), 4.41-4.38 (m, 1H), 3.83 (s, 3H), 3.76 (s, 3H), 3.17 (t, J=7.2 Hz, 2H), 2.92 (t, J=7.2 Hz, 2H), 2.55 (m, 2H), 1.69-1.59 (m, 4H).

### Example 33: Preparation of 2-(3,6-dimethoxy-5-(4,4,4-trifluorobutyl)pyridin-2-yl)ethan-1-amine (33) formate

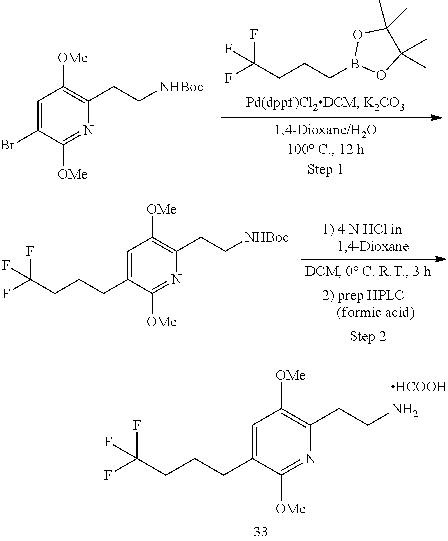 [SMILES: COc1cc(Br)c(OC)nc1CCNC(=O)OC(C)(C)C ; CC1(C)OB(CCCC(F)(F)F)OC1(C)C ; COc1cc(CCCC(F)(F)F)c(OC)nc1CCNC(=O)OC(C)(C)C ; COc1cc(CCCC(F)(F)F)c(OC)nc1CCN ; "·HCOOH"]

### Step 1a: Preparation of 4,4,5,5-tetramethyl-2-(4,4,4-trifluorobutyl)-1,3,2-dioxaborolane

To a stirred solution of anhydrous copper (1) iodide (0.100 g, 0.524 mmol) in anhydrous DMF (10 mL) under nitrogen atmosphere at room temperature was added triphenylphosphine (0.179 g, 0.681 mmol), 4,4,5,5-tetramethyl-2-(4,4,5,5-tetramethyl-1,3,2-dioxaborolan-2-yl)-1,3,2-dioxaborolane (1.9 g, 7.85 mmol), and lithium methoxide (0.398 g, 10.5 mmol). The reaction mixture was stirred at room temperature for 5 minutes, and then 4-bromo-1,1,1-trifluorobutane (1.0 g, 5.24 mmol) in anhydrous DMF (3 mL) was added dropwise under nitrogen atmosphere. The reaction mixture was stirred at room temperature for 12 h. The reaction progress was monitored by TLC and LC-MS. Once complete, the reaction mixture was quenched with water (30 mL) and extracted with ethyl acetate (2×50 mL). The combined organic extracts were washed with brine (30 mL), dried over anhydrous Na₂SO₄, filtered, and concentrated under reduced pressure to afford a crude residue that was purified by column chromatography (SiO₂, 5% Ethyl acetate/Hexane). Desired fractions were concentrated under reduced pressure to afford 4,4,5,5-tetramethyl-2-(4,4,4-trifluorobutyl)-1,3,2-dioxaborolane as a colorless liquid (Yield: 0.600 g, 48%). ¹H NMR (400 MHz, CDCl3) δ=2.27-2.06 (m, 2H), 1.81-1.65 (m, 2H), 1.22 (s, 12H), 0.84 (t, J=8 Hz, 2H).

### Step 1: Preparation of tert-butyl (2-(3,6-dimethoxy-5-(4,4,4-trifluorobutyl)pyridin-2-yl)ethyl)carbamate

A stirred solution of tert-butyl (2-(5-bromo-3,6-dimethoxypyridin-2-yl)ethyl)carbamate (0.200 g, 0.554 mmol), 4,4,5,5-tetramethyl-2-(4,4,4-trifluorobutyl)-1,3,2-dioxaborolane (0.395 g, 1.66 mmol) and potassium carbonate (0.230 g, 1.66 mmol), in 1,4-dioxane/water (5.0 mL of a 7:3 v/v mixture) under nitrogen atmosphere was heated to 100° C. for 12 h. The reaction progress was monitored by TLC and LC-MS. Once complete, the reaction mixture was cooled to room temperature and then diluted with ethyl acetate (10 mL). The mixture was passed through a Celite pad to remove the catalyst and the Celite was washed with ethyl acetate (2×10 mL). The combined organic layers were diluted with water (10 mL) and the aqueous layer was extracted with ethyl acetate (2×25 mL). The combined organic extracts were washed with brine (20 mL), dried over anhydrous Na₂SO₄, filtered, and concentrated under reduced pressure to afford a crude residue that was purified by column chromatography (SiO₂, 10% Ethyl acetate/Hexane) and desired fractions were concentrated under reduced pressure to afford tert-butyl (2-(3,6-dimethoxy-5-(4,4,4-trifluorobutyl)pyridin-2-yl)ethyl)carbamate as a white solid (Yield: 0.120 g, 55%). HRMS m/z 393.3 [M+1]⁺.

### Step 2: Preparation of 2-(3,6-dimethoxy-5-(4,4,4-trifluorobutyl)pyridin-2-yl)ethan-1-amine (33) formate

A stirred solution of tert-butyl (2-(3,6-dimethoxy-5-(4,4,4-trifluorobutyl)pyridin-2-yl)ethyl)carbamate (0.120 g, 0.306 mmol) in dichloromethane (3 mL) at 0° C. was treated with 4 N hydrogen chloride in 1,4-dioxane (3 mL) over a period of 5 min. The reaction mixture was stirred at room temperature for 3 h. After completion, the reaction mixture was concentrated under reduced pressure to afford a crude residue. The crude compound was purified by preparative HPLC using 0.1% formic acid buffer in water, desired fractions were lyophilized to afford Compound 33 formate as an off-white solid (Yield: 0.020 g, 19.89%). HRMS n/z 293.25 [M+1]⁺; ¹H NMR (400 MHz, DMSO-d₆) δ=8.41 (s, 1H), 7.33 (s, 1H), 3.82 (s, 3H), 3.76 (s, 3H), 3.06 (t, J=7.2 Hz, 2H), 2.86 (t, J=7.6 Hz, 2H), 2.60 (m, 2H), 2.32-2.20 (m, 2H), 1.79-1.71 (m, 2H).

### Example 34: Preparation of 2-(5-(5-fluoropentyl)-3,6-dimethoxypyridin-2-yl)ethan-f-amine (34) trifluoroacetate

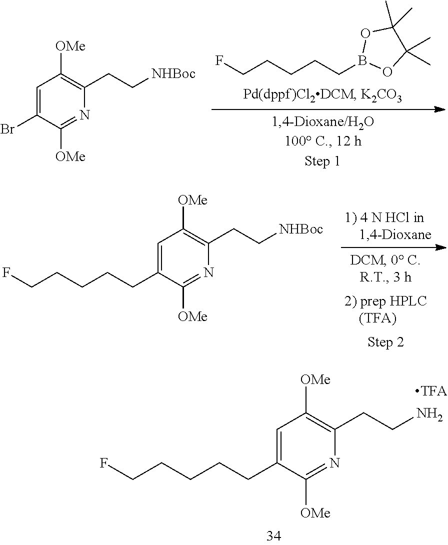 [SMILES: COc1cc(Br)c(OC)nc1CCNC(=O)OC(C)(C)C ; CC1(C)OB(CCCCCF)OC1(C)C ; COc1cc(CCCCCF)c(OC)nc1CCNC(=O)OC(C)(C)C ; COc1cc(CCCCCF)c(OC)nc1CCN ; "·TFA"]

### Step 1a: Preparation of 2-(5-fluoropentyl)-4,4,5,5-tetramethyl-1,3,2-dioxaborolane

A solution of anhydrous copper (I) iodide (56.3 mg, 0.296 mmol), triphenylphosphine (0.101 g, 0.385 mmol), 4,4,5,5-tetramethyl-2-(4,4,5,5-tetramethyl-1,3,2-dioxaborolan-2-yl)-1,3,2-dioxaborolane (1.13 g, 4.44 mmol) and lithium methoxide (0.224 g, 5.92 mmol) under nitrogen atmosphere was stirred at room temperature for 5 minutes. To the solution was added 1-bromo-5-fluoropentane (0.5 g, 2.96 mmol) in anhydrous DMF (2 mL) dropwise under nitrogen atmosphere. Reaction mixture was stirred at room temperature for 12 h. The reaction progress was monitored by TLC and LC-MS. Once complete, the reaction mixture was quenched with water (30 mL) and diluted with ethyl acetate (50 mL). The aqueous phase was extracted with ethyl acetate (2×50 mL). The combined organic extracts were washed with brine (30 mL), dried over anhydrous Na₂SO₄, filtered, and concentrated under reduced pressure to afford a crude residue that was purified by column chromatography (SiO₂, 5% Ethyl acetate/Hexane). The desired fractions were concentrated under reduced pressure to afford 2-(5-fluoropentyl)-4,4,5,5-tetramethyl-1,3,2-dioxaborolane (0.3 g, 47% yield) as a colorless liquid. ¹H NMR (400 MHz, CDCl₃) δ=4.48 (t, J=6.4 Hz, 1H), 4.36 (t, J=6.0 Hz, 1H), 1.77-1.62 (m, 2H), 1.51-1.34 (m, 4H), 1.28-1.21 (m, 12H), 0.79 (t, J=8 Hz, 2H); ¹⁹F NMR (400 MHz, CDCl₃): δ−217.91.

### Step 1: Preparation of tert-butyl (2-(5-(5-fluoropentyl)-3,6-dimethoxypyridin-2-yl)ethyl)carbamate

To a stirred solution of tert-butyl (2-(5-bromo-3,6-dimethoxypyridin-2-yl)ethyl)carbamate (0.350 g, 0.969 mmol), 2-(5-fluoropentyl)-4,4,5,5-tetramethyl-1,3,2-dioxaborolane (0.628 g, 2.91 mmol) and potassium carbonate (0.402 g, 2.91 mmol) in 1,4-dioxane/water (5.0 mL of a 7:3 v/v mixture) was purged with nitrogen for 10 min. The solution was treated with PdCl₂(dppf)·DCM (0.079 g, 0.096 mmol) under nitrogen atmosphere followed by heating to 100° C. for 12 h. The reaction progress was monitored by TLC and LC-MS. Once complete, the reaction mixture was cooled to room temperature. The mixture was diluted with ethyl acetate (20 mL), passed through Celite pad to remove the catalyst, and the Celite was washed with ethyl acetate (2×20 mL). The combined organic layers were diluted with water (20 mL) and the aqueous phase was extracted with ethyl acetate (2×25 mL). The combined organic extracts were washed with brine (20 mL), dried over anhydrous Na₂SO₄, filtered, and concentrated under reduced pressure to afford a crude residue that was purified by column chromatography (SiO₂, 10% Ethyl acetate/Hexane). Desired fractions were concentrated under reduced pressure to afford tert-butyl (2-(5-(5-fluoropentyl)-3,6-dimethoxypyridin-2-yl)ethyl)carbamate as a white solid. (Yield: 0.100 g, 28%). HRMS m/z 371.10 [M+1]⁺.

### Step 2: Preparation of 2-(5-(5-fluoropentyl)-3,6-dimethoxypyridin-2-yl)ethan-1-amine (34) trifluoroacetate

To a stirred solution of tert-butyl (2-(5-(5-fluoropentyl)-3,6-dimethoxypyridin-2-yl)ethyl)carbamate (0.100 g, 0.270 mmol) in dichloromethane (3 mL) at 0° C. was added 4 N hydrogen chloride in 1,4-dioxane (3 mL) over a period of 5 min. The reaction mixture was stirred at room temperature for 3 h. After completion, the reaction mixture was concentrated under reduced pressure to afford a crude residue that was purified by preparative HPLC using 0.1% TFA buffer in water. The desired fractions were lyophilized to afford Compound 34 trifluoroacetate as an off-white solid (Yield: 0.082 g, 83%). HRMS m/z 271.25 [M+1]⁺; ¹H NMR (400 MHz, DMSO-d₆) δ=7.73 (br s, 3H), 7.34 (s, 1H), 4.49 (t, J=6 Hz, 1H), 4.37 (t, J=6.4 Hz, 1H), 3.83 (s, 3H), 3.77 (s, 3H), 3.17 (t, J=7.6 Hz, 2H), 2.92 (t, J=7.2 Hz, 2H), 2.51 (m, 2H), 1.73-1.52 (m, 4H), 1.40-1.32 (m, 2H).

### Example 35: Preparation of 2-(3,6-dimethoxy-5-(4,4,4-trifluorobutyl)pyridin-2-yl)ethan-1-amine (35) hydrochloride

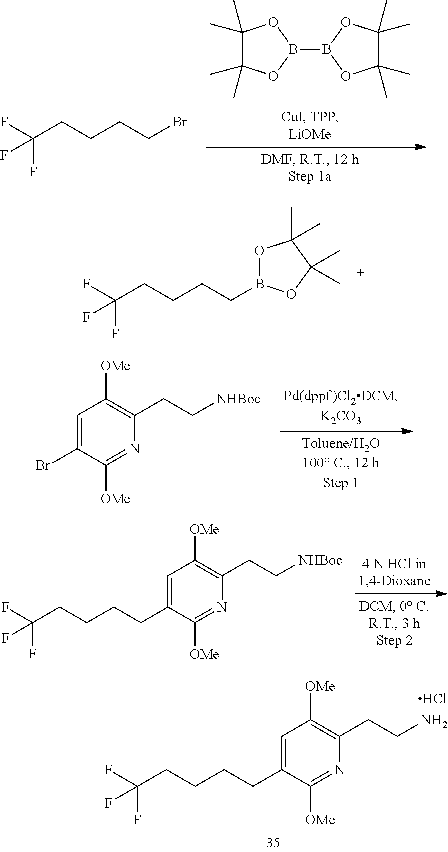

### Step 1a: Preparation of 4,4,5,5-tetramethyl-2-(5,5,5-trifluoropentyl)-1,3,2-dioxaborolane

To a stirred solution of 4,4,5,5-tetramethyl-2-(4,4,5,5-tetramethyl-1,3,2-dioxaborolan-2-yl)-1,3,2-dioxaborolane (1.86 g, 1.5 eq, 7.32 mmol) in dimethylformamide (20 mL, 258 mmol) was added LiOMe (370 mg, 2 eq, 9.76 mmol). Then reaction was purged with argon for 10 min, then 5-bromo-1,1,1-trifluoropentane (1 g, 4.88 mmol) and CuI (92.9 mg, 0.1 eq., 0.488 mmol), triphenylphosphine (166 mg, 0.13 eq., 0.634 mmol) were added. The reaction mixture stirred at R.T. for 12 h and progress of reaction was monitored by TLC. After completion of reaction, the mixture was quenched by NH₄Cl and extracted with ethyl acetate. Organic layers were dried over Na₂SO₄ and concentrated under reduced pressure to provide a crude material which was purified by flash silica chromatography. The desired product was eluted in 10% ethyl acetate in hexane to afford 4,4,5,5-tetramethyl-2-(5,5,5-trifluoropentyl)-1,3,2-dioxaborolane (0.7 g, 57% yield) as a colorless liquid. ¹H NMR (400 MHz, CDCl₃): δ=2.11-1.99 (m, 2H), 1.59-1.44 (m, 3H), 1.24 (s, 12H), 0.80 (t, J=7.6 Hz, 3H).

### Step 1: Synthesis of tert-butyl (2-(3,6-dimethoxy-5-(5,5,5-trifluoropentyl)pyridin-2-yl)ethyl)carbamate

To a stirred solution of tert-butyl (2-(5-bromo-3,6-dimethoxypyridin-2-yl)ethyl)carbamate (0.5 g, 1 eq, 1.38 mmol) in toluene (7 mL) and water (3 mL) was added 4,4,5,5-tetramethyl-2-(5,5,5-trifluoropentyl)-1,3,2-dioxaborolane (698 mg, 2 eq, 2.77 mmol) and potassium carbonate (0.574 g, 3 eq, 4.15 mmol). The reaction mixture was degassed with argon for 5 min, then PdCl₂(dppf)·DCM (56.5 mg, 0.05 eq, 0.069 mmol) was added, and the resulting reaction mixture was heated at 100° C. for 12 h. The progress of the reaction was monitored by TLC and LC-MS. After completion, the reaction mixture was diluted with water and extracted with ethyl acetate. The combined organic layers were washed with brine and dried over anhydrous Na₂SO₄, filtered, and solvent was evaporated under reduced pressure to give a crude compound which was purified by prep HPLC to afford tert-butyl (2-(3,6-dimethoxy-5-(5,5,5-trifluoropentyl)pyridin-2-yl)ethyl)carbamate (Yield: 90 mg, 16%) as a colorless liquid. LC-MS m/z 407.20 [M+1]⁺.

### Step 2: Preparation of 2-(3,6-dimethoxy-5-(5,5,5-trifluoropentyl)pyridin-2-yl)ethan-1-amine (35) hydrochloride

To a stirred solution of tert-butyl N-{2-[3,6-dimethoxy-5-(5,5,5-trifluoropentyl)pyridin-2-yl]ethyl}carbamate (90 mg, 123 μmol) in dichloromethane (3 mL) at 0° C. was added 4 N HCl in 1,4-dioxane (1 mL). The reaction was stirred at R.T. for 3 h. The progress of reaction was monitored by LC-MS. After completion, the reaction was concentrated under reduced pressure to afford a crude material which was triturated with DCM (0.2 mL) and diethyl ether (2.0 mL) followed by n-pentane (5 mL), resulting compound dried under reduced pressure to afford Compound 35 HCl as an off-white solid compound (Yield: 49 mg, 73%). LC-MS m/z 307.2 [M+1]⁺; ¹H NMR (400 MHz, DMSO-d₆) δ=7.90 (br s, 2H), 7.35 (s, 1H), 3.82 (s, 3H), 3.76 (s, 3H), 3.19-3.14 (m, 2H), 2.93 (t, J=7.2 Hz, 2H), 2.56-2.49 (m, 2H), 2.32-2.13 (m, 2H), 1.64-1.59 (m, 2H), 1.57-1.52 (m, 2H).

### Example 36: Preparation of 2-(3,6-dimethoxy-5-propylpyridin-2-yl)ethan-1-amine (36) hydrochloride

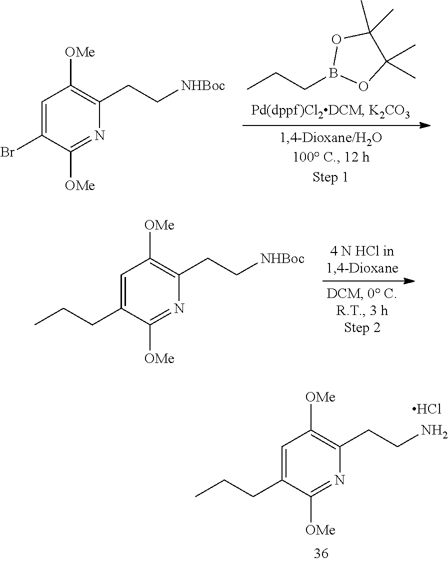 [SMILES: COc1cc(Br)c(OC)nc1CCNC(=O)OC(C)(C)C ; CCCB1OC(C)(C)C(C)(C)O1 ; CCCc1cc(OC)c(CCNC(=O)OC(C)(C)C)nc1OC ; CCCc1cc(OC)c(CCN)nc1OC ; Cl]

### Step 1: Preparation of tert-butyl (2-(3,6-dimethoxy-5-propylpyridin-2-yl)ethyl)carbamate

A stirred solution of tert-butyl (2-(5-bromo-3,6-dimethoxypyridin-2-yl)ethyl)carbamate (0.350 g, 0.969 mmol), 4,4,5,5-tetramethyl-2-propyl-1,3,2-dioxaborolane (0.330 g, 1.94 mmol) and potassium carbonate (0.402 g, 2.91 mmol) in 1,4-dioxane/water (5.0 mL of a 7:3 v/v mixture) was purged with nitrogen for 10 min. To this mixture was added PdCl₂(dppf)·DCM (0.079 g, 0.097 mmol) under nitrogen atmosphere and then the mixture was heated to 100° C. for 12 h. The reaction progress was monitored by TLC and LC-MS. Once complete, the reaction mixture was allowed cool to room temperature. The reaction was diluted with ethyl acetate (10 mL), passed through a Celite pad to remove the catalyst, then washed with ethyl acetate (2×10 mL). The combined organic layers were diluted with water (10 mL) and extracted with ethyl acetate (2×25 mL). The combined organic extracts were washed with brine (20 mL), then dried over anhydrous Na₂SO₄, filtered, and concentrated under reduced pressure to afford crude residue that was purified by column chromatography (SiO₂, 10% Ethyl acetate/Hexane) desired fraction was concentrated under reduced pressure to afford tert-butyl (2-(3,6-dimethoxy-5-propylpyridin-2-yl)ethyl)carbamate as a white solid (Yield: 0.070 g, 22%). HRMS m/z 325.10 [M+1]⁺.

### Step 2: Preparation of 2-(3,6-dimethoxy-5-propylpyridin-2-yl)ethan-1-amine (36) hydrochloride

To a stirred solution of tert-butyl (2-(3,6-dimethoxy-5-propylpyridin-2-yl)ethyl)carbamate (0.070 mg, 0.216 mmol) in dichloromethane (2 mL) at 0° C. was added 4 N hydrogen chloride in 1,4-dioxane (3 mL) over a period of 5 min. The reaction mixture was stirred at room temperature for 3 h. After completion, the reaction mixture was concentrated under reduced pressure to afford a crude residue that was triturated by using n-pentane (2 mL) and diethyl ether (1 mL) to afford Compound 36 HCl as an off-white solid (Yield: 0.050 g, 89%). HRMS m/z 225.20 [M+1]⁺; ¹H NMR (400 MHz, DMSO-d₆) δ=7.83 (br s, 3H), 7.32 (s, 1H), 3.82 (s, 3H), 3.79 (s, 3H), 3.17-3.08 (m, 2H), 2.92 (t, J=7.2 Hz, 2H), 2.46 (m, 2H), 1.59-1.50 (m, 2H), 0.89 (t, J=7.2 Hz, 3H).

### Example 37: Preparation of 1-(3,6-dimethoxy-5-propylpyridin-2-yl)butan-2-amine (37) formate

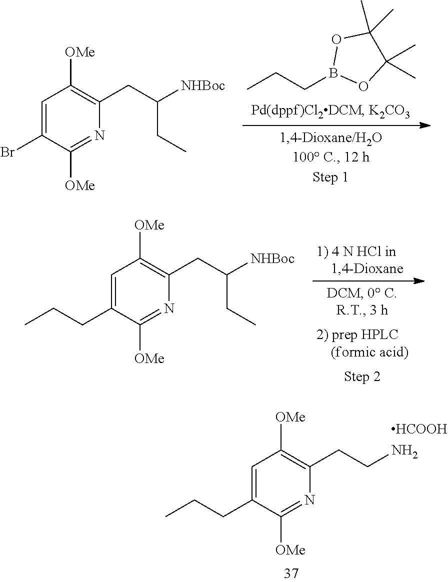 [SMILES: CCC(Cc1nc(OC)c(Br)cc1OC)NC(=O)OC(C)(C)C ; CCCB1OC(C)(C)C(C)(C)O1 ; CCCc1cc(OC)c(CC(CC)NC(=O)OC(C)(C)C)nc1OC ; CCCc1cc(OC)c(CCN)nc1OC ; "·HCOOH"]

### Step 1: Preparation of tert-butyl (1-(3,6-dimethoxy-5-propylpyridin-2-yl)butan-2-yl)carbamate

A stirred solution of tert-butyl (1-(5-bromo-3,6-dimethoxypyridin-2-yl)butan-2-yl)carbamate (0.26 g, 0.668 mmol), 4,4,5,5-tetramethyl-2-propyl-1,3,2-dioxaborolane (0.227 g, 1.34 mmol) and potassium carbonate (0.277 g, 2.0 mmol) in 1,4-dioxane/water (5.0 mL of a 7:3 v/v mixture) was purged with nitrogen for 10 min. The solution was treated with PdCl₂(dppf)·DCM (0.027 g, 0.066 mmol) under nitrogen atmosphere and then heated to 100° C. for 12 h. The reaction progress was monitored by TLC and LC-MS. Once complete, the reaction mixture was cooled to room temperature and then diluted with ethyl acetate (10 mL). The mixture was passed through Celite pad to remove the catalyst and the aqueous phase was washed with ethyl acetate (2×10 mL). The combined organic layer was diluted with water (15 mL) and extracted with ethyl acetate (2×25 mL). The combined organic extracts were washed with brine (15 mL), dried over anhydrous Na₂SO₄, filtered, and concentrated under reduced pressure to afford the crude residue that was purified by column chromatography (SiO₂, 10% Ethyl acetate/Hexane) and desired fractions were concentrated under reduced pressure to afford tert-butyl (1-(3,6-dimethoxy-5-propylpyridin-2-yl)butan-2-yl)carbamate as a white solid (Yield: 0.090 g, 38%). HRMS m/z 353.30 [M+1]⁺.

### Step 2: Preparation of 1-(3,6-dimethoxy-5-propylpyridin-2-yl)butan-2-amine (37) formate

To a stirred solution of tert-butyl (1-(3,6-dimethoxy-5-propylpyridin-2-yl)butan-2-yl)carbamate (0.090 g, 0.255 mmol) in dichloromethane (2 mL) was added 4 N hydrogen chloride in 1,4-dioxane (3 mL) at 0° C. over a period of 5 min. The reaction mixture was stirred at room temperature for 3 h. After completion, the reaction mixture was concentrated under reduced pressure to afford a crude residue that was purified by preparative HPLC using 0.1% formic acid buffer in water and desired fractions were lyophilized to afford Compound 37 formate as an off-white solid (Yield: 0.031 g, 48%). HRMS m/z 253.25 [M+1]⁺; ¹H NMR (400 MHz, DMSO-d₆) δ=8.39 (s, 1H), 7.29 (s, 1H), 3.81 (s, 3H), 3.75 (s, 3H), 3.51-3.25 (m, 1H), 2.86-2.81 (m, 1H), 2.76-2.72 (m, 1H), 2.45 (m, 2H), 1.59-1.49 (m, 2H), 1.48-1.41 (m, 2H), 0.899 (t, J=7.2 Hz, 6H).

### Example 38: Preparation of 1-(5-hexyl-3,6-dimethoxypyridin-2-yl)propan-2-amine (38) hydrochloride

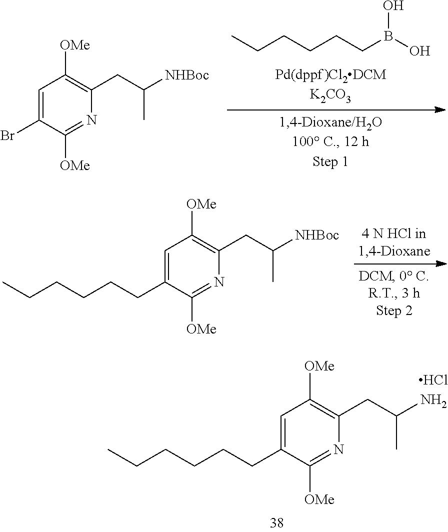

### Step 1: Preparation of tert-butyl (1-(5-hexyl-3,6-dimethoxypyridin-2-yl)propan-2-yl)carbamate

A stirred solution of tert-butyl (1-(5-bromo-3,6-dimethoxypyridin-2-yl)propan-2-yl)carbamate (0.2 g, 0.533 mmol), n-hexylboronic acid (0.138 g, 1.06 mmol) and potassium carbonate (0.221 g, 1.60 mmol) in 1,4-dioxane/water (5.0 mL of a 7:3 v/v mixture) was purged with nitrogen for 10 min. To this solution was added PdCl₂(dppf)·DCM (0.043 g, 0.053 mmol) under nitrogen atmosphere and the mixture was heated to 100° C. for 12 h. The reaction progress was monitored by TLC and LC-MS. Once complete, the reaction mixture was cooled to room temperature and then diluted with ethyl acetate (10 mL). The mixture was passed through a Celite pad to remove the catalyst and the Celite was washed with ethyl acetate (2×10 mL). The combined organic layers were diluted with water (10 mL) and extracted with ethyl acetate (2×25 mL). The combined organic extracts were washed with brine (20 mL), dried over anhydrous Na₂SO₄, filtered, and concentrated under reduced pressure to afford a crude residue that was purified by column chromatography (SiO₂, 10% Ethyl acetate/Hexane). Desired fractions were concentrated under reduced pressure to afford tert-butyl (1-(5-hexyl-3,6-dimethoxypyridin-2-yl)propan-2-yl)carbamate as a white solid (Yield: 0.051 g, 25%). HRMS m/z 381.20 [M+1]⁺.

### Step 2: Preparation of 1-(5-hexyl-3,6-dimethoxypyridin-2-yl)propan-2-amine (38) hydrochloride

To a stirred solution of tert-butyl (1-(5-hexyl-3,6-dimethoxypyridin-2-yl)propan-2-yl)carbamate (0.051 mg, 1 eq, 0.134 mmol) in dichloromethane (2 mL) at 0° C. was added 4 N HCl in 1,4-dioxane (2 mL) over a period of 5 min. The reaction mixture was stirred at room temperature for 3 h. After completion, the reaction mixture was concentrated under reduced pressure to afford crude residue that was triturated by using n-pentane (2 mL) and diethyl ether (1 mL) to afford Compound 38 HCl as an off-white solid (Yield: 0.035 g, 88%). HRMS m/z 281.35 [M+1]⁺; ¹H NMR (400 MHz, DMSO-d₆) δ=7.88 (br s, 3H), 7.33 (s, 1H), 3.84 (s, 3H), 3.76 (s, 3H), 2.92 (m, 2H), 2.51-2.49 (m, 2H), 1.54 (m, 2H), 1.30-1.25 (m, 6H), 1.19 (d, J=6.4 Hz, 3H), 0.83 (t, J=6.4 Hz, 3H).

### Example 39: Preparation of Phenyl Comparator Compounds

The phenyl comparator compounds shown below were prepared according to previously described procedures (WO2022192781A1):

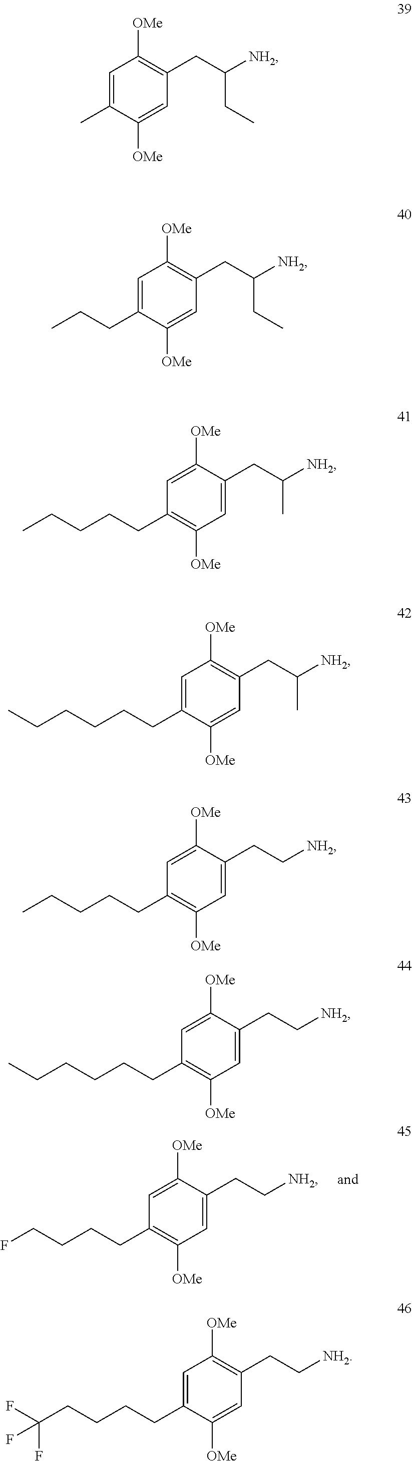

### Example 40: 5-HT2A Receptor Binding

The binding affinities of disclosed compounds at the ketanserin binding site of the 5-HT2A receptor were determined in radioligand binding experiments, with the results summarized in Table 1. Disclosed compounds exhibited substantial binding affinity for the 5-HT2A receptor. The affinity of Compound 1 was much higher than the other pyridine isomer Compound 11 and the pyrazine Compound 12, indicating the preferred positioning of the pyridine nitrogen in the compounds of the invention. Generally, compounds with an ethyl substituent alpha to the basic amine were less potent than compounds bearing a hydrogen or methyl substituent at this position. Longer alkyl or fluoroalkyl substituents at position 5 of the pyridine also tended to increase potency compared to smaller substituents at this position.

TABLE 1
5-HT2A receptor binding affinities of disclosed compounds.

| | Kᵢ at 5-HT2A |
|---|---|
| | Receptor (nM) |
| Compound | ([³H]ketanserin) |
| DOI | 14.5 |
| 25D-NBOMe | 2.52 |
| 2C-TFM | 15.8 |
| Mescaline | 17,400 |
| 2C-B | 8.25 |
| 2C-E | 16.7 |
| 1 | 40.0 |
| 4 | 26.18 |
| 5 | 1167 |
| 6 | 167.5 |
| 7 | 6.32 |
| 8 | 1120 |
| 9 | 99.16 |
| 10 | 26.3 |
| 11 | 273 |
| 12 | 693 |
| 16 | 263.9 |
| 17 | 404.1 |
| 18 | 1200 |
| 19 | 111.3 |
| 20 | 321.9 |
| 22 | 82.79 |
| 23 | 344.3 |
| 24 | 649.1 |
| 25 | 0.35 |
| 26 | 150.15 |
| 27 | 303.8 |
| 29 | 0.91 |
| 30 | 6.01 |
| 31 | 19.68 |
| 32 | 103.3 |
| 33 | 104.2 |
| 34 | 10.19 |
| 35 | 13.62 |
| 36 | 35.38 |
| 37 | 106.25 |
| 38 | 3.1 |
| 39 | 1473.95 |
| 40 | 58.59 |
| 41 | 5.41 |
| 42 | 2.03 |
| 43 | 7.10 |
| 44 | 0.70 |
| 45 | 57.43 |
| 46 | 6.31 |

### Methods:

5-HT2A Receptor Radioligand Binding. DOI, 25D-NBOMe, 2C-TFM, mescaline, 2C-B, and 2C-E were commercially obtained. Other compounds were prepared as described above. Affinities of the test compounds for the 5-HT2A receptor were determined in radioligand binding experiments with [³H]ketanserin by WuXi AppTec (Hong Kong) Limited, using methods adapted from the literature and under conditions described in Table 2.

TABLE 2
Assay conditions for 5-HT2A receptor radioligand binding.

| Receptor Source | HEK293 stable cell line | |
|---|---|---|
| Vehicle | 1.0% DMSO | |
| Incubation Time | 1 | h |
| Incubation Temperature | 25° | C. |

| | |
|---|---|
| Incubation Buffer | 50 mM Tris-HCl, pH 7.4 |
| Ligand | 1 nM [³H]ketanserin |
| Non-Specific Ligand | 1 μM ketanserin |

### Example 41: Functional Activity at 5-HT2A, 5-HT2B, 5-HT2C, and 5-HT1A Receptors

The functional activity of disclosed compounds at several 5-HT receptor subtypes (5-HT2A, 5-HT2B, 5-HT2C, and 5-HT1A) was determined in Ca²⁺ flux assays, with the results summarized in Table 3. Reference phenalkylamine compounds tested exhibited high efficacy agonist activity at the 5-HT2A receptor. The novel pyridine Compound 1 demonstrated only 25% activation at max dose, which was much lower efficacy than the 72% activation exhibited by its phenyl counterpart Compound 10. This trend toward lower maximal efficacy at the 5-HT2A receptor was also observed for other disclosed pyridine compounds, including Compounds 2, 37, 29, 32, and 35, compared to their phenyl counterparts, Compounds 39, 40, 44, 45, and 46, respectively. Accordingly, these pyridine compounds represent low efficacy partial agonists that may exhibit attenuated hallucinogenic effects compared to 5-HT2A agonists of greater efficacy. In contrast, the other pyridine isomer, Compound 11, was both less potent and exhibited greater efficacy than Compound 1. Further, the pyrazine analog, Compound 12, possessed very lower potency and efficacy. These observations support the preferred positioning of the pyridine nitrogen in the compounds of the invention. At the 5-HT2B receptor, a number of the disclosed pyridine compounds, including Compounds 4, 38, 7, 9, 19, 29, 32, and 35, showed lower maximal efficacy than their phenyl counterparts, Compounds 41, 42, 43, 2C—B, 2C-E, 44, 45, and 46, respectively, suggesting that they may have improved cardiovascular safety. The disclosed pyridine compounds also uniformly showed little agonist activity at the 5-HT1A receptor (EC₅₀ greater than 10 μM), demonstrating the high selectivity of these compounds for agonism of 5-HT2A receptors over 5-HT1A receptors.

TABLE 3
Functional activity of disclosed compounds at 5-HT receptor subtypes.

| | 5-HT2A | % Act | 5-HT2B | % Act @ | 5-HT2C | % Act | 5-HT1A | % Act |
|---|---|---|---|---|---|---|---|---|
| | EC₅₀ | @ Max | EC₅₀ | Max | EC₅₀ | @ Max | EC₅₀ | @ Max |
| Compound | (nM) | Conc. | (nM) | Conc. | (nM) | Conc. | (nM) | Conc. |
| DOI | 0.190 | 106 | 4.88 | 86.3 | 0.818 | 92.6 | >10,000 | −2.47 |
| 25D- | 0.251 | 97.5 | 1.27 | 43.3 | <0.508 | 91.5 | >10,000 | −4.43 |
| NBOMe | | | | | | | | |
| 2C-TFM | 1.05 | 81.5 | 16.3 | 82.0 | 0.593 | 88.2 | >10,000 | −0.647 |
| Mescaline | 114 | 102 | 853 | 45.4 | 536 | 91.1 | 12,500 | −5.03 |
| 2C-B | 0.478 | 99.7 | 20.0 | 73.3 | 1.14 | 89.4 | 33,100 | 47.8 |
| 2C-E | 0.747 | 105 | 29.7 | 66.2 | 1.82 | 89.5 | 43,700 | 17.9 |
| 1 | 115 | 25.3 | NC | 0.18 | 5474 | 44.38 | 20,200 | 188 |
| 2 | 847.4 | 69.78 | NT | NT | NT | NT | NT | NT |
| 3 | 136.0 | 70.17 | NT | NT | NT | NT | NT | NT |
| 4 | 15.45 | 132.71 | >10,000 | 14.53 | NT | NT | 48148 | 67.31 |
| 5 | 30.04 | 104.25 | 574.7 | 68.46 | NT | NT | >100,000 | 0.59 |
| 6 | 2.135 | 95.6 | 170.4 | 70.54 | NT | NT | >100,000 | 1.66 |
| 7 | 19.96 | 82.88 | >10,000 | 11.56 | 16.16 | 92.53 | 51,931 | 61.95 |
| 8 | 65.8 | 85.81 | 1,005 | 36.99 | NT | NT | >100,000 | −0.62 |
| 9 | 9.81 | 96.84 | 764 | 38.01 | 30.72 | 95.94 | >100,000 | 2.11 |
| 10 | 36.6 | 71.5 | NC | 0.211 | 1,820 | 115 | >10,000 | 33.1 |
| 11 | 356 | 62.2 | NC | 6.92 | NT | NT | 19,200 | 197 |
| 12 | >3,160 | 37.1 | NC | 7.05 | NT | NT | 33,300 | 170 |
| 16 | 20.14 | 97.33 | 1396 | 38.95 | NT | NT | >100,000 | 5.31 |
| 17 | 6.27 | 99.4 | 539.6 | 70.03 | NT | NT | >100,000 | 0.79 |
| 18 | 369.7 | 70.92 | >10,000 | 2.75 | 2254 | 36.76 | >100,000 | 1.33 |
| 19 | 20.25 | 96.03 | 217.9 | 20.07 | NT | NT | 12,005 | 1.25 |
| 20 | 5.79 | 98.11 | 292.8 | 75.75 | NT | NT | >100,000 | 0.9 |
| 21 | 170.5 | 73.61 | NT | NT | NT | NT | NT | NT |
| 22 | 4.09 | 94.73 | 214.8 | 22.75 | NT | NT | >100,000 | −1.51 |
| 23 | 3.03 | 106.2 | 44.46 | 67.46 | NT | NT | >100,000 | 3.21 |
| 24 | 74.58 | 77.03 | >10,000 | 14.99 | NT | NT | >100,000 | 2.99 |
| 25 | 1.592 | 93.57 | >10,000 | 8.22 | NT | NT | >100,000 | 21.35 |
| 26 | 27.04 | 81.58 | >10,000 | 5.16 | NT | NT | >100,000 | 2.22 |
| 27 | 15.0 | 104.13 | 245.9 | 61.18 | NT | NT | >100,000 | 0.49 |
| 28 | 161.2 | 71.49 | NT | NT | NT | NT | NT | NT |
| 29 | 34.58 | 69.91 | >10,000 | 7.78 | NT | NT | >100,000 | NT |
| 30 | 64.96 | 73.21 | 1673 | 23.14 | NT | NT | 91,557 | 55.61 |
| 31 | 7.88 | 90.63 | >10,000 | 7.29 | NT | NT | >100,000 | 21.32 |
| 32 | 20.78 | 81.86 | >10,000 | −0.69 | 22.79 | 99.77 | >100,000 | 0.5 |
| 33 | 45.66 | 68.5 | >10,000 | 1.05 | NT | NT | >100,000 | 10.46 |
| 34 | 13.54 | 64.66 | >10,000 | 7.93 | NT | NT | >100,000 | 4.8 |
| 35 | 38.47 | 48.95 | 340.8 | 32.44 | NT | NT | >100,000 | 41.47 |
| 36 | 4.14 | 88.19 | 184.0 | 21.53 | NT | NT | >100,000 | 3.03 |
| 37 | 128.4 | 49.58 | >10,000 | 12.18 | NT | NT | >100,000 | 42.24 |
| 38 | 11.45 | 83.97 | >10,000 | 4.76 | 158.7 | 79.49 | 56,897 | 123.88 |
| 39 | 48.92 | 112.8 | 1391 | 39.2 | 288.3 | 99.3 | >10,000 | 8.7 |
| 40 | 10.9 | 84.23 | >10,000 | −0.18 | 19.22 | 93.58 | 63,542 | 96.43 |
| 41 | 1.953 | 100.2 | 42.02 | 20.0 | 3.75 | 90.1 | >10,000 | 15.0 |
| 42 | 2.65 | 119.7 | 294.0 | 39.8 | 6.34 | 99.8 | >10,000 | 30.4 |
| 43 | 3.34 | 105.2 | 258.6 | 62.2 | 1.65 | 85.6 | >10,000 | 18.9 |
| 44 | 2.09 | 90.51 | 716.2 | 46.79 | 5.51 | 82.62 | 30,453 | 183.21 |
| 45 | 1.40 | 95.03 | 472.6 | 54.09 | 0.662 | 99.28 | >10,000 | 9.36 |
| 46 | 12.49 | 80.79 | 19.59 | 77.62 | 6.41 | 82.10 | 30,538 | 78.98 |

NT = not tested;
NC = not calculated due to very low efficacy

### Methods:

Functional Assays at 5-HT2A, 5-HT2B, and 5-HT1A Receptors. DOI, 25D-NBOMe, 2C-TFM, mescaline, 2C-B, and 2C-E were commercially obtained. Other compounds were prepared as described above. Agonist activity at 5-HT2A, 5-HT2B, and 5-HT1A receptors was determined using a FLIPR Ca²⁺ flux assay at WuXi AppTec (Hong Kong) Limited according to their standard protocols. Briefly, stably transfected cells expressing the receptor of interest (HEK293 for 5-HT2A and 5-HT2B; CHO cells for 5-HT1A) were grown and plated in 384-well plates and incubated at 37° C. and 5% CO₂ overnight. A solution of 250 mM probenecid in 1 mL FLIPR assay buffer was prepared fresh. This was combined with a fluorescent dye (Fluo-4 DirectTM) to make a final assay concentration of 2.5 mM. Compounds were diluted 1:3.16 for 10 points and 750 nL was added to a 384-well compound plate using ECHO along with 30 μL assay buffer. The fluorescent dye was then added to the assay plate along with assay buffer to a final volume of 40 μL. The cell plate was incubated for 50 min at 37° C. and 5% CO₂ and placed into the FLIPR Tetra along with the compound plate. 10 μL of references and compounds were then transferred from the compound plate into the cell plate and the fluorescent signal was read.

Functional Assays at 5-HT2C Receptors. Agonist activity at 5-HT2C receptors was determined using a FLIPR Ca²⁺ flux assay at Eurofins DiscoverX (Fremont, CA) according to their standard protocols. Briefly, stably transfected cells expressing the human 5-HT2C receptor were grown and plated in a 384-well plate and incubated at 37° C. and 5% CO₂ overnight. Assays were performed in 1× Dye Loading Buffer consisting of 1× Dye, 1× Additive A, and 2.5 mM Probenecid in HBSS/20 mM Hepes. Probenecid was prepared fresh. Cells were loaded with dye prior to testing and incubated at 37° C. for 30-60 minutes. After dye loading, cells were removed from the incubator and 10 μL HBSS/20 mM Hepes was added. 3× vehicle was included in the assay buffer. Cells were incubated for 30 mins at room temperature in the dark to equilibrate plate temperature. Intermediate dilution of sample stocks was performed to generate 4× sample in assay buffer. Compound agonist activity was measured on a FLIPR Tetra (MDS). Calcium mobilization was monitored for 2 minutes and 10 μL 4× sample in HBSS/20 mM Hepes was added to the cells 5 seconds into the assay.

### Example 42: Effects on the Head Twitch Response (HTR) in Mice

Compounds were tested for their ability to induce a head twitch response (HTR) in mice, with the results summarized in Table 4. Agonists of the 5-HT2A receptor are well known to induce this effect in rodents and the potency of this HTR is correlated with hallucinogenic potency in humans. The control 5-HT2A receptor agonist DOI exhibited a high Eₘₐₓ in this assay, consistent with the strong hallucinogenic effects of this compound reported in humans. In contrast, Compound 1 showed an attenuated Eₘₐₓ in the HTR, suggestive of an attenuated hallucinogenic effect and consistent with the partial agonist activity of the compound in vitro.

TABLE 4
Activity of disclosed compounds in the manual HTR assay in mice.

| | ED₅₀ | Eₘₐₓ |
|---|---|---|
| Compound | (mg/kg) | (HTR counts/20 min) |
| DOI | 0.428 | 35.7 |
| 1 | 0.897 | 18.78 |

### Methods:

Animals. Adult male C57BL/6 mice aged 8-10 weeks (body weight 20-25 g) were used in these experiments. Animals were housed under controlled temperatures and 12-hour light/dark cycles (lights on between 07:00-19:00 h), with ad libitum food and water. The protocol was approved by the Institutional Animal Care and Use Committee at the study site. This study was carried out in strict accordance with the recommendations in the Guide for the Care and Use of Laboratory Animals of the National Institutes of Health. All efforts were made to minimize suffering.

Drugs and Drug administration. DOI was commercially obtained. All other compounds were synthesized as described above. All drugs were administered subcutaneously (SC) dissolved in saline vehicle (or saline acidified with 1-2 molar equivalents HCl to form the salt in situ for freebase compounds) and at a volume of 10 mL/kg. Drugs were administered at 4 or 5 doses per compound, using n=6 animals/group. Doses were calculated on the basis of the freebase form, except for DOT, which was calculated based on the HCl salt.

Procedure. Mice were administered one dose of the drug (or vehicle) SC and immediately placed into a small open field for behavioral observation. Animals were observed continuously for 20 mins and the number of head twitches (HTs) were counted by an observer blind to the treatment condition.

Statistical Analysis. Analysis was performed using GraphPad Prism 9. Dose-response curves were fit via non-linear regression using the Gaussian 2020 function in Prism in order to determine the ED₅₀ and Eₘₐₓ for each compound.

### Example 43: Effects on the Head Twitch Response (HTR) in Mice Using an Automated Procedure

Disclosed compounds were tested for their ability to induce a head twitch response (HTR) in mice using an automated video tracking procedure, with the results summarized in Table 5. The disclosed pyridine compounds generally induced reduced maximal HTR compared to the control 5-HT2A agonist DOI, suggesting that the disclosed compounds may exhibit attenuated hallucinogenic effects. Compounds with longer alkyl or fluoroalkyl substituents at position 5 of the pyridine typically exhibited lower maximal HTR compared to compounds with shorter chains or otherwise smaller substituents at this position. The phenyl Compound 44 was substantially more efficacious at inducing HTR than its pyridine counterpart Compound 29, in agreement with the generally lower maximal efficacy of the disclosed pyridine compounds for signaling through 5-HT2A receptors.

TABLE 5
Activity of disclosed compounds in
the automated HTR assay in mice.

| | | | # HTR/20 min at | |
|---|---|---|---|---|
| | | | Most Efficacious | |
| | | | Dose (Most | |
| | ED50 | Emax | Efficacious Dose; | |
| Compound | (mg/kg) | (HTR counts/20 min) | mg/kg) | |
| DOI | 0.531 | 82.02 | 81.5 | (3.2) |
| 18 | NC | NC | 33.12 | (10) |
| 29 | 7.464 | 22.11 | 23 | (3.2) |
| 30 | 5.873 | 16.80 | 17.5 | (3.2) |
| 31 | 1.290 | 43.42 | 43.38 | (3.2) |
| 36 | 0.972 | 48.79 | 48.12 | (10) |
| 7 | 4.877 | 24.61 | 23.25 | (1) |
| 9 | 1.382 | 49.65 | 45.75 | (3.2) |
| 44 | 1.357 | 57.87 | 58.0 | (3.2) |

NC = not calculated due to poor curve fit

### Methods:

Animals. Adult male C57BL/6 mice aged 8-10 weeks (body weight 20-25 g) were used in these experiments. Animals were housed under controlled temperatures and 12-hour light/dark cycles (lights on between 07:00-19:00 h), with ad libitum food and water. The protocol was approved by the Institutional Animal Care and Use Committee at the study site. This study was carried out in strict accordance with the recommendations in the Guide for the Care and Use of Laboratory Animals of the National Institutes of Health. All efforts were made to minimize suffering.

Drugs and Drug Administration. DOT was commercially obtained. Disclosed compounds were synthesized as described above. They were administered subcutaneously (SC) dissolved in saline (or saline acidified with 1-2 molar equivalents HCl to form the salt in situ for freebase compounds) at a volume of 10 mL/kg. Five doses were tested, with N=5-8 animals/group. Doses were calculated on the basis of the freebase, except for DOI, which was calculated based on the HCl salt.

Procedure. Mice were administered one dose of the drug and immediately placed into a small open field for behavioral observation. High-speed video of animals' behavior was captured continuously for 20 min. Video recordings were imported into an image processing software, which detects multiple points of interest on each animal's body, including the left and right ear. HTR events were defined as periodic deviations of ear locations from temporally averaged ear locations. Predefined thresholds for magnitude and frequency of location deviations were used to filter 1-FR events from random head movements. The method was validated by comparing detected events with manual (human) observations and found to have an accuracy >95%. The total number of HTR events counted during the 20-min recording were determined for each animal at each dose.

Statistical Analysis. Analysis was performed using GraphPad Prism 9. Dose-response curves were fit via non-linear regression using the Gaussian 2020 function in Prism in order to determine the ED₅₀ and Eₘₐₓ for each compound.

The described embodiments of the present invention are intended to be illustrative rather than restrictive and are not intended to represent every embodiment of the present invention. Various modifications and variations can be made without departing from the spirit or scope of the invention as set forth in the following claims, both literally and in equivalents recognized in law.

What is claimed is:

1. A compound of the structure: 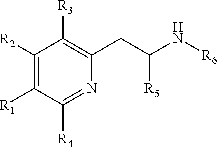
or a pharmaceutically acceptable salt thereof, wherein
R₁ is hydrogen, C₁-C₈ alkyl, C₂-C₈ alkenyl, C₂-C₈ alkynyl, C₃-C₁₀ cycloalkyl, 3- to 6-membered heterocyclyl, C₃-C₁₀ cycloalkyl C₁-C₈ alkyl, 3- to 6-membered heterocyclyl C₁-C₈alkyl, —OR₇, —SR₇, halo, —CF₃, —SF₅, —OCF₃, —CN, —NO₂, —NR₈R₉, aryl, heteroaryl, aryl C₁-C₈ alkyl, or heteroaryl C₁-C₈ alkyl;
R₂ is hydrogen, C₁-C₈ alkyl, C₂-C₈ alkenyl, C₂-C₈ alkynyl, C₃-C₁₀ cycloalkyl, C₃-C₁₀ cycloalkyl C₁-C₃ alkyl, —OR₁₀, —SR₁₀, halo, —CF₃, —SF₅, —OCF₃, —CN, —NO₂, or —NR₁₁R₁₂;
R₃ is hydrogen, C₁-C₈ alkyl, C₂-C₈ alkenyl, C₂-C₈ alkynyl, C₃-C₁₀ cycloalkyl, C₃-C₁₀ cycloalkyl C₁-C₈alkyl, —OR₁₃, —SR₁₃, halo, —CF₃, —SF₅, —OCF₃, —CN, —NO₂, —NR₁₄R₁₅, —C(O)R₁₆, —C(O)OR₁₆, —O—C(O)R₁₆, or —C(O)NR₁₇R₁₈;
R₄ is hydrogen, C₁-C₈ alkyl, C₂-C₈ alkenyl, C₂-C₈ alkynyl, C₃-C₁₀ cycloalkyl, C₃-C₁₀ cycloalkyl C₁-C₈alkyl, —OR₁₉, —SR₁₉, halo, —CF₃, —SF₅, —OCF₃, —CN, —NO₂, —NR₂₀R₂₁, —C(O)R₂₂, —C(O)OR₂₂, —O—C(O)R₂₂, or —C(O)NR₂₃R₂₄;
R₅ is hydrogen, C₁-C₃ alkyl, C₂-C₃ alkenyl, C₂-C₃ alkynyl, C₁-C₃ alkoxy, cyclopropyl, or cyclopropylmethyl; and
R₆ is hydrogen or benzyl, wherein the phenyl ring of benzyl is optionally substituted with 1-5 instances of R₆ₐ and each R₆ₐ is independently selected for each occurrence from the group consisting of hydroxyl, C₁-C₅ alkyl, C₂-C₅ alkenyl, C₂-C₅ alkynyl, C₃-C₁₀ cycloalkyl, C₃-C₁₀ cycloalkyl C₁-C₅ alkyl, halo, —CF₃, —SF₅, —OCF₃, —OR₂₅, —SR₂₅, —CN, —NO₂, —NR₂₆R₂₇, —C(O)R₂₈, —C(O)OR₂₈, —O—C(O)R₂₈, and —C(O)NR₂₉R₃₀, or wherein any two adjacent R₆ₐ can be taken together with the atoms on which they are attached to form a C₄-C₆ cycloalkyl or 4- to 6-membered heterocyclyl;
wherein
R₇ is hydrogen, C₁-C₈ alkyl, C₂-C₈ alkenyl, C₂-C₈ alkynyl, C₃-C₁₀ cycloalkyl, 3- to 6-membered heterocyclyl, C₃-C₁₀ cycloalkyl C₁-C₈ alkyl, 3- to 6-membered heterocyclyl C₁-C₈ alkyl, aryl, heteroaryl, aryl C₁-C₈ alkyl, or heteroaryl C₁-C₈ alkyl;
R₈ and R₉ are independently hydrogen, C₁-C₈ alkyl, C₂-C₈ alkenyl, C₂-C₈ alkynyl, C₃-C₁₀ cycloalkyl, 3- to 6-membered heterocyclyl, C₃-C₁₀ cycloalkyl C₁-C₈ alkyl, 3- to 6-membered heterocyclyl C₁-C₈ alkyl, aryl, heteroaryl, aryl C₁-C₈ alkyl, or heteroaryl C₁-C₈ alkyl;
R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, and R₃₀ are independently hydrogen, C₁-C₅ alkyl, C₂-C₅ alkenyl, C₂-C₅ alkynyl, C₃-C₆ cycloalkyl, 3- to 6-membered heterocyclyl, C₃-C₆ cycloalkyl C₁-C₅ alkyl, or 3- to 6-membered heterocyclyl C₁-C₅ alkyl;
aryl is a monocyclic or bicyclic aromatic ring containing 6 or 10 ring carbon atoms;
heteroaryl is a 5- to 10-membered ring, which is either monocyclic or bicyclic, containing 1, 2, 3, or 4 ring heteroatoms and 2 to 9 ring carbon atoms;
aryl and heteroaryl groups may be unsubstituted or substituted with one or more substituents independently selected from hydroxyl, C₁-C₅ alkyl, C₂-C₅ alkenyl, C₂-C₅ alkynyl, C₃-C₆ cycloalkyl, C₁-C₅ alkoxy, halo, —CF₃, —CN, —NO₂, —NH₂, aryl, 3- to 6-membered heterocyclyl, heteroaryl, C₃-C₆ cycloalkyl C₁-C₃ alkyl, aryl C₁-C₃ alkyl, 3- to 6-membered heterocyclyl C₁-C₃ alkyl, and heteroaryl C₁-C₃ alkyl;
cycloalkyl and heterocyclyl groups may be unsubstituted or substituted with one or more substituents independently selected from hydroxyl, C₁-C₅ alkyl, C₂-C₅ alkenyl, C₂-C₅ alkynyl, C₃-C₆ cycloalkyl, C₁-C₅ alkoxy, halo, —CF₃, —CN, —NO₂, —NH₂, aryl, 3- to 6-membered heterocyclyl, heteroaryl, C₃-C₆ cycloalkyl C₁-C₃ alkyl, aryl C₁-C₃ alkyl, 3- to 6-membered heterocyclyl C₁-C₃ alkyl, and heteroaryl C₁-C₃ alkyl; and
alkyl, alkenyl, and alkynyl groups may be unsubstituted or substituted with one or more substituents independently selected from hydroxyl, C₃-C₆ cycloalkyl, C₁-C₅ alkoxy, halo, —CF₃, —CN, —NO₂, —NH₂, aryl, 3- to 6-membered heterocyclyl, heteroaryl, C₃-C₆ cycloalkyl C₁-C₃ alkyl, aryl C₁-C₃ alkyl, 3- to 6-membered heterocyclyl C₁-C₃ alkyl, and heteroaryl C₁-C₃ alkyl;
or a pharmaceutically acceptable salt thereof.

2. The compound according to claim 1, wherein heteroaryl and aryl groups may be unsubstituted or substituted with one or more substituents independently selected from the group consisting of hydroxyl, C₁-C₅ alkyl, C₂-C₅ alkenyl, C₂-C₅ alkynyl, C₃-C₅ cycloalkyl, C₁-C₅ alkoxy, halo, —CF₃, —CN, —NH₂, and —NO₂;
alkyl, alkenyl, and alkynyl groups may be unsubstituted or substituted with one or more substituents independently selected from the group consisting of fluoro, C₃-C₅ cycloalkyl, hydroxyl, and C₁-C₅ alkoxy; and
cycloalkyl and 3-6 membered heterocyclyl may be unsubstituted or substituted with one or more substituents independently selected from the group consisting of fluoro, C₁-C₅ alkyl, hydroxyl, and C₁-C₅ alkoxy.

3. The compound according to claim 1, wherein
R₂ is hydrogen, hydroxyl, C₁-C₃ alkyl, halo, —CF₃, —SF₅, —OCF₃, —CN, —NO₂, —NH₂, —NH(C₁-C₃ alkyl), —N(C₁-C₃ alkyl)₂, C₁-C₃ alkoxy, or —S(C₁-C₃ alkyl);
R₃ is hydrogen, hydroxyl, —OR₁₃, —SR₁₃, —C(O)NH₂, or —O—C(O)R₁₆;
R₄ is hydroxyl, —OR₁₉, —SR₁₉, —C(O)NH₂, or —O—C(O)R₂₂;
R₅ is hydrogen or C₁-C₂ alkyl; and
R₆ is hydrogen or benzyl, wherein the phenyl ring of benzyl is optionally substituted with 1-5 instances of R₆ₐ and each R₆ₐ is independently selected for each occurrence from the group consisting of hydroxyl, C₁-C₃ alkyl, C₂-C₃ alkenyl, C₂-C₃ alkynyl, C₃-C₅ cycloalkyl, halo, —CF₃, —SF₅, —OCF₃, C₁-C₃ alkoxy, —S(C₁-C₃ alkyl), —CN, —NO₂, —NH₂, —NH(C₁-C₃ alkyl), —N(C₁-C₃ alkyl)₂, —C(O)(C₁-C₃ alkyl), —C(O)O(C₁-C₃ alkyl), —O—C(O)(C₁-C₃ alkyl), —C(O)NH₂, —C(O)NH(C₁-C₃ alkyl), and —C(O)N(C₁-C₃ alkyl)₂, or wherein any two adjacent R₆ₐ can be taken together with the atoms on which they are attached to form an optionally substituted 4-6-membered cycloalkyl or 4-6-membered heterocyclyl, wherein said heterocyclyl contains 1 or 2 ring heteroatoms selected from oxygen, nitrogen, and sulfur.

4. The compound according to claim 1, wherein
R₁ is C₁-C₈ alkyl, C₂-C₈ alkenyl, C₂-C₈ alkynyl, C₃-C₈ cycloalkyl, C₃-C₆ cycloalkyl C₁-C₅ alkyl, —OR₇, —SR₇, halo, —CF₃, —SF₅, —OCF₃, —CN, —NO₂, or —NR₈R₉;
R₂ is hydrogen or C₁-C₃ alkoxy;
R₃ is hydrogen or C₁-C₃ alkoxy;
R₄ is C₁-C₃ alkoxy;
R₅ is hydrogen or C₁-C₂ alkyl;
R₆ is hydrogen or benzyl, wherein the phenyl ring of benzyl is optionally substituted with 1-5 instances of R₆ₐ and each R₆ₐ is independently selected for each occurrence from the group consisting of hydroxyl, C₁-C₃ alkyl, halo, and C₁-C₃ alkoxy, or wherein any two adjacent R₆ₐ can be taken together with the atoms on which they are attached to form an optionally substituted 4-5-membered cycloalkyl or 4-5-membered heterocyclyl; and
R₇, R₈, and R₉ are independently selected from the group consisting of hydrogen, C₁-C₈ alkyl, C₂-C₈ alkenyl, C₂-C₈ alkynyl, C₃-C₈ cycloalkyl, and C₃-C₆ cycloalkyl C₁-C₅ alkyl.

5. The compound according to claim 1, wherein
R₁ is C₁-C₈ alkyl, —S(C₁-C₈ alkyl), halo, —CF₃, —SF₅, —OCF₃, —CN, —NO₂, —NH₂, —NH(C₁-C₄ alkyl), —N(C₁-C₄ alkyl)₂;
R₂ is hydrogen or methoxy;
R₃ is hydrogen or methoxy;
R₄ is methoxy;
R₅ is hydrogen or C₁-C₂ alkyl; and
R₆ is hydrogen or benzyl, wherein the phenyl ring of benzyl is optionally substituted with 1-5 instances of R₆ₐ and each R₆ₐ is independently selected for each occurrence from the group consisting of hydroxyl, halo, and methoxy, or wherein any two adjacent R₆ₐ can be taken together with the atoms on which they are attached to form an optionally substituted 5-membered cycloalkyl or 5-membered heterocyclyl.

6. The compound according to claim 1,
wherein R₁ is C₁-C₈ alkyl, —S(C₁-C₈ alkyl), halo, or —CF₃.

7. The compound according to claim 1, wherein
R₁ is C₁-C₈ alkyl, —S(C₁-C₈ alkyl), halo, —CF₃, or —SF₅;
R₂ is hydrogen or methoxy;
R₃ is hydrogen or methoxy;
R₄ is methoxy;
R₅ is hydrogen or C₁-C₂ alkyl; and
R₆ is hydrogen or benzyl, wherein the phenyl ring of benzyl is optionally substituted with 1-2 instances of R₆ₐ and each R₆ₐ is independently selected for each occurrence from the group consisting of hydroxyl, fluoro, and methoxy, or any two adjacent R₆ₐ can be taken together with the atoms on which they are attached to form a methylenedioxy ring.

8. The compound according to claim 1, wherein R₁ is C₁-C₆ alkyl, —S(C₁-C₆ alkyl), halo, or —CF₃.

9. The compound according to claim 1, wherein R₂ is hydrogen.

10. The compound according to claim 1, wherein R₃ and R₄ are methoxy.

11. The compound according to claim 1, wherein R₅ is hydrogen, methyl, or ethyl.

12. The compound according to claim 1, wherein R₆ is hydrogen.

13. The compound according to claim 1, wherein R₆ is benzyl, wherein the phenyl ring of benzyl is optionally substituted with 1-2 instances of R₆ₐ and each R₆ₐ is independently selected for each occurrence from the group consisting of hydroxyl, fluoro, and methoxy, or wherein any two adjacent R₆ₐ can be taken together with the atoms on which they are attached to form a methylenedioxy ring.

14. The compound according to claim 1, wherein all heteroaryl, aryl, alkyl, alkenyl, alkynyl, cycloalkyl, and heterocyclyl groups are unsubstituted.

15. The compound according to claim 1, wherein the compound has the structure:

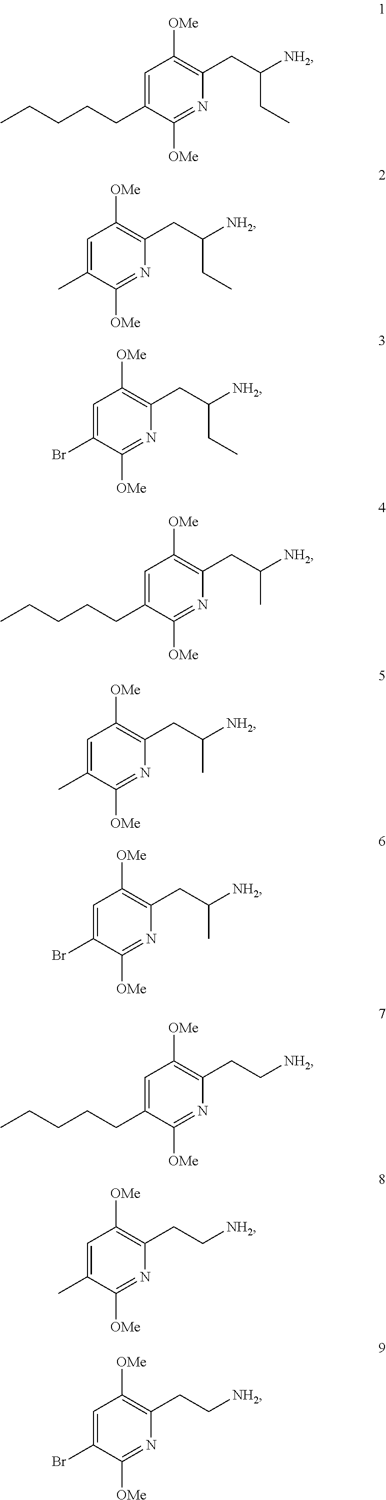

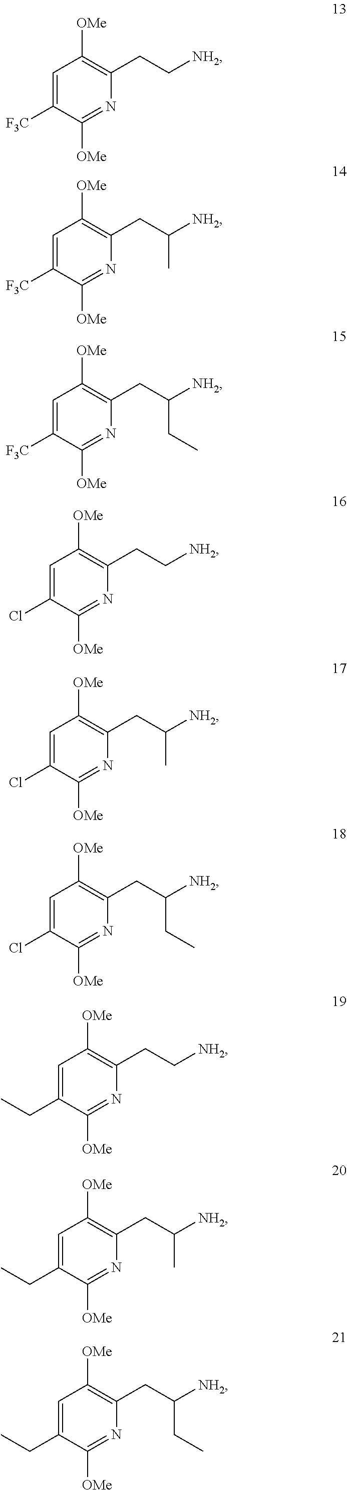

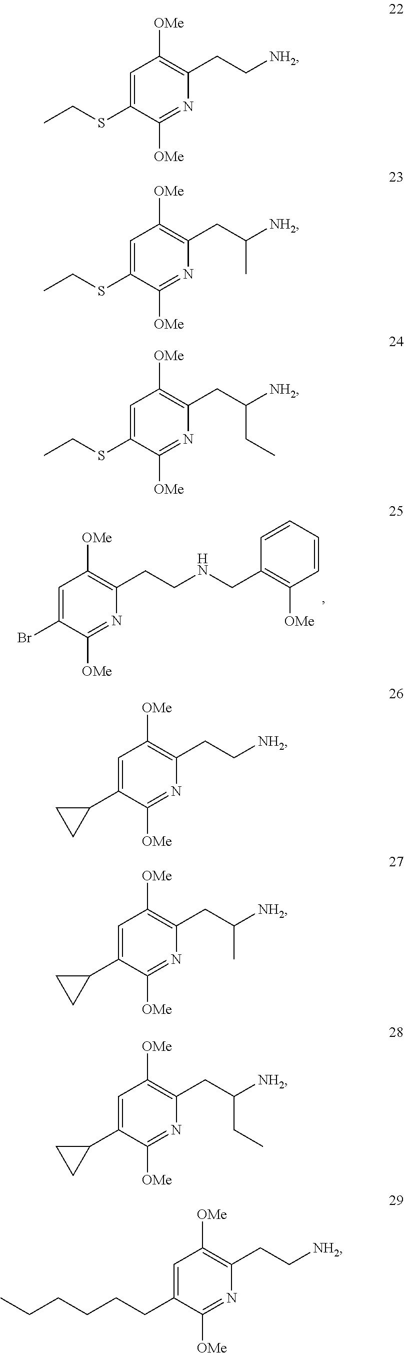

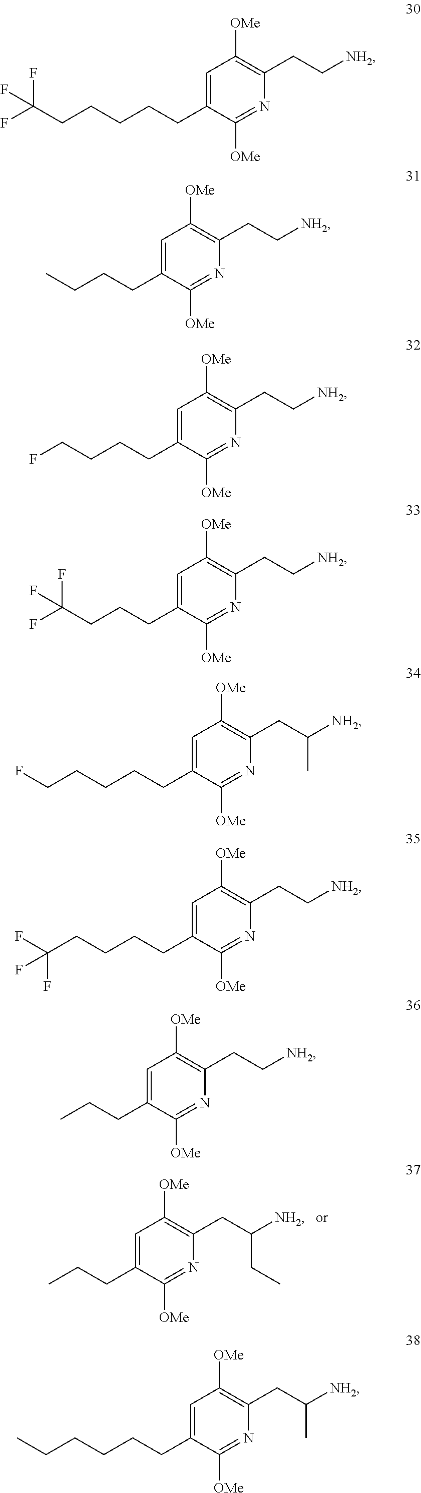

16. A pharmaceutical composition comprising a pharmaceutically effective amount of a compound according to claim 1 and a pharmaceutically acceptable carrier thereof.

17. A method of treating a psychiatric disorder in a subject comprising administering to said subject an effective amount of a compound according to claim 1.

18. The method according to claim 17 wherein the psychiatric disorder is a depressive disorder, an anxiety disorder, or a substance use disorder and any symptom or disorder associated therewith

19. A method of activating a 5-HT2A receptor in a cell comprising effecting the administration to said cell of an effective amount of a compound according to claim 1.
